(19) United States
(12) Patent Application Publication
(10) Pub. No.: US 2026/0191848 A1
(43) Pub. Date: Jul. 9, 2026

(54) METHODS OF REVERSING PARP-RESISTANCE IN CANCER

(71) Applicant: THE TRUSTEES OF COLUMBIA UNIVERSITY IN THE CITY OF NEW YORK, New York, NY (US)

(72) Inventors: Swarnali Acharyya, New York, NY (US); Anup Kumar Biswas, New York, NY (US); Yifan Tai, New York, NY (US); Angela Chow, New York, NY (US); Courtney Coker, New York, NY (US); Wanchao Ma, New York, NY (US)

(21) Appl. No.: 19/550,689

(22) Filed: Feb. 26, 2026

Related U.S. Application Data
(63) Continuation of application No. PCT/US2024/047791, filed on Sep. 20, 2024.
(60) Provisional application No. 63/584,153, filed on Sep. 20, 2023.

(51) Int. Cl. A61K 31/4439; A61K 31/5025; A61P 35/00; C12N 9/22; C12N 15/11; G01N 33/575

(52) U.S. Cl. CPC A61K 31/4439; A61K 31/5025; A61P 35/00; C12N 9/226; C12N 15/11; G01N 33/5758; C12N 2310/20; G01N 2440/14

(57) ABSTRACT

PARP inhibitor (PARPi) resistance continues to undermine successful treatment of BRCA-mutant breast cancers in the clinic. Two immunocompetent, orthotopic models of Brca1- and Bard1-mutant breast cancer were generated to explore PARPi resistance mechanisms in vivo. In contrast to the expected DNA-repair-related resistance mechanisms, the models identified activation of FLT1 (VEGFR1) as the basis for PARPi resistance. PARPi-resistant breast tumors with increased expression of FLT1 have increased expression of the FLT1 ligand PIGF in the tumor microenvironment, which activates FLT1 and its downstream effector AKT. Genetic inhibition of Flt1 inhibits AKT activation and causes dramatic tumor regression of PARPi-resistant tumors, while axitinib, a pan-VEGFR antagonist, resensitizes PARPi-resistant breast tumors to PARP inhibition in vivo. It was also discovered that breast cancers expressing high levels of FLT1 before PARPi treatment show faster progression on PARPi. Thus, novel cancer therapeutic compositions and methods related to preventing or reversing PARPi resistance are described herein.

### RELATED APPLICATIONS

This application is a bypass continuation of International Application No. PCT/US2024/047791 filed Sep. 20, 2024, which claims the benefit of U.S. provisional patent application 63/584,153, filed Sep. 20, 2023, to The Trustees of Columbia University, titled “METHODS OF REVERSING PARP-RESISTANCE IN CANCER,” the entirety of the disclosures of which are hereby incorporated by this reference.

### STATEMENT REGARDING FEDERALLY SPONSORED RESEARCH OR DEVELOPMENT

This invention was made with government support under W81XWH-21-1-0764 and W81XWH-17-1-0055 awarded by the Medical Research and Development Command. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 6,265 byte.XML file named “181WO-PCT_SeqList” created on Sep. 9, 2024.

### TECHNICAL FIELD

This document relates to methods of treating or reversing PARP inhibitor (PARPi) resistance in a cancer by blockade of FLT1 (VEGFR1), which results in suppression of AKT activation, increase in tumor infiltration of CD8⁺ T cells, and dramatic regression of PARPi-resistant tumors in a T cell-dependent manner.

### BACKGROUND

The tumor suppressor genes breast cancer 1 (BRCA1) and breast cancer 2 (BRCA2) maintain cellular genomic integrity through homologous recombination (HR)-mediated DNA repair¹⁻⁴. Individuals harboring germline mutations in either BRCA1 or BRCA2 (collectively denoted as “BRCA” hereafter) display increased susceptibility to chromosomal instability due to defective DNA repair⁵⁻⁸, which increases the lifetime risk of developing breast cancer by up to 70%^{9,10}. In addition to germline mutations, somatic mutations in BRCA are also detected in breast cancer patients^{11,12}. BRCA1-mutated tumors, and a subset of BRCA2-mutated tumors, often present as triple-negative breast cancer (TNBC), which is associated with a poor prognosis and high likelihood of recurrence^{13,14}.

Since BRCA-mutated cancer cells are deficient in HR-mediated DNA repair, they rely heavily on the single-strand break repair protein poly-ADP ribose polymerase (PARP) for repair of their DNA¹. The discovery of synthetic lethality between PARP inhibition and BRCA mutations ultimately led to the rapid clinical translation of PARP inhibitors (PARPis) for the treatment of BRCA-mutant breast cancer^{15,16}. Dramatic initial responses to PARPis, such as olaparib and talazoparib, in BRCA-mutant breast cancer patients led to the FDA approval of these drugs as monotherapyl^{7,18}. However, responses were short-lived and usually resulted in lethal recurrences¹⁹, warranting the search for mechanisms of PARPi resistance.

BRCA-mutant tumors most commonly harbor single-nucleotide mutations, small deletions, or insertions in the BRCA gene that shift the reading frame and abolish wild-type BRCA protein expression⁸. On-target secondary genetic mutations that restore the BRCA reading frame underlie the development of PARPi resistance in 50% of breast cancer patients²⁰. Additionally, genetically engineered mouse models of Brca1 deficiency have shown that non-genetic mechanisms such as the transient upregulation of the drug efflux transporter P-glycoprotein ABCB1 contribute to PARPi resistance^{21,22}. Despite progress in this area, reversing acquired PARPi resistance in the clinic has been unsuccessful and remains an unmet need.

### SUMMARY

As disclosed herein, FLT1 activation in cancer cells drives PARP-inhibitor resistance in breast cancer by activating pro-survival pathways and dampening the cytotoxic immune response. The disclosure reveals a previously unexplored role for FLT1 as an important driver of PARPi resistance and a vulnerability in BRCA-mutant breast cancers that can be pharmacologically targeted to reverse PARPi resistance in the clinic.

To identify new targetable mechanisms of PARPi resistance in the context of BRCA1 loss of function, Applicants generated orthotopic allograft mouse models using cancer cells isolated from conditional Brca1- or Bard1-deficient genetically engineered mouse models (GEMMs)²³. The BRCA1 protein functions in HR-mediated DNA repair through its heterodimerization with BRCA1-associated ring domain 1 (BARD1)^{24,25}. Genetic inactivation of either Brca1 or Bard1 in mammary epithelial cells leads to the development of triple-negative carcinomas that have similar latency, frequency, and cytogenetic features and are indistinguishable by histopathology²³.

Applicants chose to study PARPi resistance using the Brca1- and Bard1-deficient allograft models for two main reasons. First, Brca1 or Bard1 loss of function in mammary cancer cells renders them sensitive to PARP inhibition and subsequently prone to developing PARPi resistance^{17,26-30}. Therefore, the Brca1- and Bard1-deficient mammary tumor models represent in-vivo treatment models to study PARPi response and the gradual recurrence of tumors due to acquired PARPi resistance. Second, it is often overlooked that PARPi treatment can impact both innate and adaptive immunity³¹⁻³⁵. As such, most studies investigating PARPi resistance use either cell lines or subcutaneous tumors (xenografts) in immunocompromised mice, which does not allow for the analysis of PARPi-resistance mechanisms in the relevant physiological or immune context. Analogous to breast cancer patients with BRCA1 mutations, the Brca1- and Bard1-deficient PARPi-treatment models show remarkable initial responses to PARPi that are characterized by a window of treatment response followed by tumor recurrence in all mice. Consistent with the acquired resistance phenotype, PARPi-resistant tumors are no longer sensitive to PARPi treatment in vivo. Interestingly, the PARPi-resistant tumor cells still maintain their sensitivity to PARPi in vitro, suggesting that adaptive mechanisms operating locally protect these cells from PARP inhibition in vivo. In contrast to on-target genetic resistance mechanisms, Applicants identified a therapeutically targetable vulnerability in PARPi-resistant cells driven by FLT1/VEGFR1 signaling. Applicants show that the cancer cells of PARPi-resistant tumors from Brca1- and Bard1-deficient models and breast cancer patients have increased FLT1 activation. FLT1 signaling in cancer cells protects them from PARPi-induced death by activating AKT pro-survival pathways and by dampening the cytotoxic immune response. Of translational relevance, both genetically and pharmacologically blocking FLT1 re-sensitizes PARPi-resistant tumors to PARPi treatment. As such, the Brca1- and Bard1-deficient allograft models described herein can be used to screen for agents that can restore PARP sensitivity in tumor cells. One such agent identified is axitinib, a VEGFR antagonist.

In one aspect, the present invention provides for a method of treating cancer in a subject, the method comprising: administering to the subject a PARP inhibitor; and administering to the subject a therapeutic for inhibiting VEGFR activity. In certain embodiments, the therapeutic for inhibiting VEGFR activity is a FLT1/VEGFR antagonist. In certain embodiments, the therapeutic for inhibiting VEGFR activity is axitinib. In certain embodiments, the PARP inhibitor is talazoparib. In certain embodiments, the therapeutic for inhibiting VEGFR activity is a a CRISPR/Cas system or RNAi system that genetically inhibits Flt1 expression. In certain embodiments, the cancer is breast cancer, ovarian cancer, pancreatic cancer, or prostate cancer. In certain embodiments, the cancer comprises tumor cells that are deficient in a homologous recombination (HR) pathway. In certain embodiments, the cancer is a BRCA-mutant cancer. In certain embodiments, the cancer is sensitive to a PARP inhibitor. In certain embodiments, the cancer has increased expression of VEGFR1. In certain embodiments, the cancer has increased expression of placental growth factor (PIGF).

In another aspect, the present invention provides for a method of reversing PARP inhibitor resistance in a tumor cell, the method comprising inhibiting a signaling pathway initiated by FLT1/VEGFR1 in the tumor cell. In certain embodiments, the signaling pathway initiated by FLT1/VEGFR1 is inhibited by administering to the tumor cell an AKT antagonist. In certain embodiments, the signal pathway initiated by FLT1/VEGFR1 is inhibited by genetically inhibiting Flt1 expression in the tumor cell. In certain embodiments, Flt1 expression in the tumor cell is genetically inhibited using a CRISPR/Cas system or RNAi system. In certain embodiments, the signal pathway initiated by FLT1/VEGFR1 is inhibited by a degrader system. In certain embodiments, the signal pathway initiated by FLT1/VEGFR1 is inhibited by administering to the tumor cell a VEGFR antagonist. In certain embodiments, the VEFFR antagonist is axitinib.

In another aspect, the present invention provides for a method of inducing cytotoxic immune response to cancer cells in a subject, the method comprising: administering to the subject a PARP inhibitor; and administering to the subject a therapeutic for inhibiting VEGFR1 activity. In certain embodiments, the therapeutic for inhibiting VEGFR activity is a FLT1/VEGFR antagonist. In certain embodiments, the therapeutic for inhibiting VEGFR activity is axitinib. In certain embodiments, the PARP inhibitor is talazoparib.

In certain embodiments, the method further comprises administering immune checkpoint blockade therapy.

In another aspect, the present invention provides for a method of detecting PARP inhibitor resistance in tumor cells comprising detecting in a tumor sample obtained from a subject in need thereof activity of a signaling pathway initiated by FLT1/VEGFR1. In certain embodiments, the protein in the signaling pathway initiated by FLT1/VEGFR1 is VEGFR1, detecting an increase in VEGFR1 expression as compared to a reference level indicates PARP inhibitor resistance in the tumor cells. In certain embodiments, the protein in the signaling pathway initiated by FLT1/VEGFR1 is PIGF, detecting an increase in PIGF expression as compared to a reference level indicates PARP inhibitor resistance in the tumor cells. In certain embodiments, the protein in the signaling pathway initiated by FLT1/VEGFR1 is AKT, detecting phosphorylated AKT indicates PARP inhibitor resistance in the tumor cells. In certain embodiments, the subject is treated according to any embodiment herein if a cancer resistant to a PARP inhibitor is detected.

In another aspect, the present invention provides for a method of treating cancer in a subject, the method comprising: administering to the subject a PARP inhibitor; providing a biological sample obtained from the subject, wherein the biological sample is obtained after the subject has been administered the PARP inhibitor; detecting activity of a signaling pathway initiated by FLT1/VEGFR1, wherein expression or phosphorylation of a protein in the signaling pathway initiated by FLT1/VEGFR1 is detected, thereby detecting cancer resistant to a PARP inhibitor in the subject; and administering to the subject a therapeutic for inhibiting VEGFR activity upon detection of presence of PARP inhibitor resistant tumor cells in the subject.

In another aspect, the present invention provides for a method of treating cancer in a subject, the method comprising: obtaining a first biological sample from the subject; detecting activity of a signaling pathway initiated by FLT1/VEGFR1 in the first biological sample, wherein a first expression or phosphorylation level of a protein in the signaling pathway initiated by FLT1/VEGFR1 is detected; administering to the subject a PARP inhibitor; obtaining a second biological sample from the subject, wherein the second biological sample is obtained after the subject has been administered the PARP inhibitor; detecting activity of a signaling pathway initiated by FLT1/VEGFR1 in the second biological sample, wherein a second expression or phosphorylation level of the protein in the signaling pathway initiated by FLT1/VEGFR1 is detected and wherein an increase in expression or phosphorylation level of the protein in the signaling pathway initiated by FLT1/VEGFR1 in the second biological sample compared to the first biological sample indicates presence of cancer cells resistant to the PARP inhibitor in the subject; and administering to the subject a therapeutic for inhibiting VEGFR activity upon detection of presence of cancer cells resistant to the PARP inhibitor in the subject. In certain embodiments, the protein in the signaling pathway initiated by FLT1/VEGFR1 is VEGFR1, detecting an increase in VEGFR1 expression in the second biological sample compared to the first biological sample indicates PARP inhibitor resistance in the subject's cancer. In certain embodiments, the protein in the signaling pathway initiated by FLT1/VEGFR1 is PIGF, detecting an increase in PIGF expression in the second biological sample compared to the first biological sample indicates PARP inhibitor resistance in the subject's cancer. In certain embodiments, the protein in the signaling pathway initiated by FLT1/VEGFR1 is AKT, detecting an increase in phosphorylated AKT in the second biological sample compared to the first biological sample indicates PARP inhibitor resistance in the subject's cancer. In certain embodiments, the first biological sample and the second biological sample are from a tumor.

In another aspect, the present invention provides for a method of treating cancer in a subject, the method comprising: obtaining a tumor sample from the subject; detecting FLT1/VEGFR1 expression in the tumor sample; and administering a therapeutic for inhibiting VEGFR activity and a PARP inhibitor to the subject upon detection of increased expression of FLT1/VEGFR1 compared to a reference expression level. In certain embodiments, the therapeutic for inhibiting VEGFR activity is a FLT1/VEGFR antagonist. In certain embodiments, the therapeutic for inhibiting VEGFR activity is axitinib. In certain embodiments, the PARP inhibitor is talazoparib.

In certain embodiments, the method further comprises administering to the subject immune checkpoint blockade therapy.

In another aspect, the present invention provides for a method of screening for a therapeutic agent that reverses PARP inhibitor resistance in a tumor cell comprising: administering to the tumor cell a PARP inhibitor; detecting in the tumor cell activity of a signaling pathway initiated by FLT1/VEGFR1 after the tumor cell has been administered the PARP inhibitor, wherein expression or phosphorylation of a protein in the signaling pathway initiated by FLT1/VEGFR1 is detected to produce a first FLT1/VEGFR1 signaling pathway activity level and increased expression or phosphorylation of the protein in the signaling pathway initiated by FLT1/VEGFR1 in the first FLT/VEGFR1 signaling pathway activity level compared to a reference value indicates the development of PARP inhibitor resistance in the tumor cell; administering to tumor cell a potential therapeutic agent; detecting in the tumor cell activity of the signaling pathway initiated by FLT1/VEGFR1 after the tumor cell has been administered the potential therapeutic agent, wherein expression or phosphorylation of a protein in the signaling pathway initiated by FLT1/VEGFR1 is detected to produce a second FLT/VEGFR1 signaling pathway activity level and reduced expression or phosphorylation of the protein in the signaling pathway initiated by FLT1/VEGFR1 in the second FLT/VEGFR1 signaling pathway activity level compared to the first FLT/VEGFR1 signaling pathway activity level indicates the potential therapeutic agent reverses PARP inhibitor resistance.

In another aspect, the present invention provides for a method of screening for agents that reverse PARP inhibitor resistance in a tumor cell as shown and described herein.

In another aspect, the present invention provides for a method of identifying cancer therapeutic agents for combination therapy with PARP inhibitor as shown and described herein.

In another aspect, the present invention provides for a screening platform for studying tumor cell resistance to PARP inhibitor treatment, the screening platform comprising: a Brca1-deficient orthotopic allograft model; or a Bard1-deficient orthotopic allograft model.

The foregoing and other aspects, features, and advantages will be apparent from the DESCRIPTION and DRAWINGS, and from the CLAIMS if any are included. Reference is also made to Tai Y, Chow A, Han S, et al. FLT1 activation in cancer cells promotes PARP-inhibitor resistance in breast cancer. EMBO Mol Med. 2024; 16 (8): 1957-1980, which is incorporated by reference herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations will hereinafter be described in conjunction with the appended and/or included DRAWINGS, where like designations denote like elements.

FIG. 1A-FIG. 1F—Generation of PARPi (talazoparib) resistance in Brca1-def and Bard1-def breast tumor models. FIG. 1A, Schematic representation of the in vivo treatment regimen designed to generate talazoparib-resistant, Brca1-def breast cancer cell lines. Mice were injected with luciferase-labeled, Brca1-def breast cancer cells that had not previously been exposed to talazoparib and therefore remained sensitive to this drug. Following the development of tumors, mice with similar tumor sizes were then randomized into either vehicle (“Veh”) or talazoparib (“Tal”) treatment groups. Treatment with Tal (0.3 mg/kg body weight per day, five days/week, oral gavage) began two weeks following tumor-cell injection. Tumors were collected after tumor relapse in the Tal-treated group and were then dissociated and selected to generate Tal-resistant Brca1-def breast cancer cell lines. FIG. 1B, Relapse in Brca1-def Tal-sensitive (“Tal-Sen”) tumors occurred about seven weeks following the start of Tal treatment, after which tumor growth increased despite continued drug treatment, thus indicating the acquisition of Tal resistance. Tal-resistant, Brca1-def tumors were then isolated at 13 weeks following tumor-cell injection. n=32 for Veh-treated mice; n=24 for Tal-treated mice. Data are presented as mean values±SEM. P values were determined by a two-tailed, unpaired, Mann-Whitney test. * at 2 weeks indicates P=0.0169; *** at 3 weeks indicates P=0.0007; **** at 5 and 7 weeks indicates P<0.0001. FIG. 1C, Mice were injected with the Tal-resistant (“Tal-Res”), Brca1-def breast cancer cell line isolated in B and then checked for sensitivity to Tal. n=21 for Veh-treated mice; n=31 for Tal-treated mice. P values were determined by a two-tailed, unpaired, Mann-Whitney test. * at 3 weeks indicates P=0.0174; ns: not significant. FIG. 1D, Schematic representation of in vivo treatment regimen to generate the Tal-resistant, Bard1-def breast cancer cell line, which was performed with similar methodologies to those described in A. Mice were injected with luciferase-labeled, Tal-sensitive, Bard1-def breast cancer cells. Following the development of tumors, mice with similar tumor sizes were randomized into Veh or Tal treatment groups. Treatment began one-week post tumor-cell injection. FIG. 1E, Tal-resistant, Bard1-def relapse tumors, which arose at about three weeks post Brca1-def Tal-Sen tumor-cell injection, were isolated at five weeks post tumor-cell injection. n=5 for Veh-treated mice; n=6 for Tal-treated mice. Data are presented as mean values±SEM. P values were determined by a two-tailed, unpaired, Mann-Whitney test. ** at 3 and 4 weeks indicates P=0.0043; ** at 5 weeks indicates P=0.0095. FIG. 1F, Mice were injected with the Tal-Res, Bard1-def breast cancer cell line isolated in E and then checked for sensitivity to Tal. n=12 for Veh-treated mice; n=14 for Tal-treated mice. P values were determined by a two-tailed, unpaired, Mann-Whitney test. ns: not significant.

FIG. 2A-FIG. 2L—Talazoparib (Tal)-resistant tumors show increased KDR/VEGFR2 and PIGF expression but only modest sensitization to Tal upon VEGFR2 depletion. FIG. 2A, Representative images of immunohistochemistry (IHC) for CD31⁺ on talazoparib-sensitive (“Sen”) and -resistant (“Res”) tumors from the Brca1-def and Bard1-def models described in FIG. 1. FIG. 2B, Immunostained tumor sections from A were quantified using automated QuPath software to identify positively stained cells. n=4 for both Sen and Res tumors for both models. Data are presented as mean values±SEM. P values were determined by a two-tailed, unpaired, Mann-Whitney test. * indicates P=0.0286 for both models. FIG. 2C, Representative images of IHC for VEGFA on Sen and Res tumor sections from the Brca1-def and Bard1-def models as described in A. FIG. 2D, Immunostained tumor sections from C were quantified using automated QuPath software to identify positively stained cells. For the Brca1-def model, n=5 for both Sen and Res tumors. For the Bard1-def model, n=5 for Sen tumors, and n=4 for Res tumors. Data are presented as mean values±SEM. P values were determined by a two-tailed, unpaired, Mann-Whitney test. * indicates P=0.0317 for the Bard1-def model; ns: not significant. FIG. 2E, Representative images of IHC for PIGF on Sen and Res tumor sections from the Brca1-def and Bard1-def models described in A. FIG. 2F, Immunostained tumor sections from E were quantified using automated QuPath software to identify positively stained cells. For the Brca1-def model, n=5 for both Sen and Res tumors. For the Bard1-def model, n=5 for Sen tumors, and n=4 for Res tumors. Data are presented as mean values±SEM. P values were determined by a two-tailed, unpaired, Mann-Whitney test. ** indicates P=0.0079 for the Brca1-def model, and * indicates P=0.0159 for the Bard1-def model. FIG. 2G, Representative images of IHC for VEGFR2 (KDR) on Sen and Res tumor sections from the Brca1-def and Bard1-def models described in A. FIG. 2H, Immunostained tumor sections from G were quantified using automated QuPath software to identify positively stained cells. n=4 for both Sen and Res tumors for both models. Data are presented as mean values±SEM. P values were determined by a two-tailed, unpaired, Welch's test. ** indicates P=0.003 for the Brca1-def model, and * indicates P=0.0249 for the Bard1-def model. FIG. 2I, Schematic representation of the experiment designed to test the effect of in vivo inhibition of VEGFR2 in Res tumors from the Brca1-def and Bard1-def models. For the Brca1-def model, Res cancer cells were injected into mice, and two weeks later, randomized tumor-bearing mice received one of the following four treatments: 1) Isotype control antibody (anti-horseradish peroxidase)+Vehicle (“Isotype+Veh”), 2) Isotype+Tal, 3) anti-mouse VEGFR2 antibody (“Anti-VEGFR2”)+Veh, and 4) Anti-VEGFR2+Tal. Brca1-def mice were then euthanized for tumor collection four weeks post Res-cancer-cell injection. For the Bard1-def model, mice started receiving treatments one-week post Res-cancer-cell injection and were euthanized three weeks post Res-cancer cell injection. FIG. 2J, Tumor growth curves for the Res-tumor-bearing mice described in I. For the Brca1-def model, n=4 for the Isotype+Veh group, n=4 for the Isotype+Tal group, n=4 for the Anti-VEGFR2+Veh group, and n=5 for the Anti-VEGFR2+Tal group. In the Bard1-def model, n=3 for the Isotype+Veh group, n=6 for the Isotype+Tal group, n=3 for the Anti-VEGFR2+Veh group, and n=7 for the Anti-VEGFR2+Tal group. Data are presented as mean values±SEM. P values were determined by a two-tailed, unpaired, Student's t-test, comparing endpoint tumor volumes between the Isotype+Tal and Anti-VEGFR2+Tal groups. For the Brca1-def model, * at 4 weeks indicates P=0.0174. For the Bard1-def model, * at 3 weeks indicates P=0.0306. FIG. 2K, Representative images of tumors following the treatment regimens from J. FIG. 2L, Tumor weights from J were plotted after collection at endpoint. For the Brca1-def model, n=4 for the Isotype+Veh group, n=4 for the Isotype+Tal group, n=4 for the Anti-VEGFR2+Veh group, and n=5 for the Anti-VEGFR2+Tal group. For the Bard1-def model, n=3 for Isotype+Veh group, n=6 for Isotype+Tal group, n=3 for Anti-VEGFR2+Veh group, and n=7 for Anti-VEGFR2+Tal group. Data are presented as mean values±SEM. P values were determined by a two-tailed, unpaired, Student's t-test, comparing endpoint tumor weights between the Isotype+Tal and Anti-VEGFR2+Tal groups. * indicates P=0.0141 for the Brca1-def model, and * indicates P=0.0222 for the Bard1-def model.

FIG. 3A-FIG. 3H—FLT1/VEGFR1 expression in cancer cells promotes talazoparib resistance in breast cancer models and is associated to shorter progression-free survival (PFS) in breast cancer patients. FIG. 3A, Representative images of IHC for pFLT1 IHC on sections from the talazoparib-sensitive (“Sen”) and -resistant (“Res”) Brca1-def and Bard1-def breast cancer tumors from FIG. 1. FIG. 3B, Immunostained sections from A were quantified using automated QuPath software to identify positively stained cells. n=4 for both Sen and Res tumors from both models. Data are presented as mean values±SEM. P values were determined by a two-tailed, unpaired, Mann-Whitney test. * indicates P=0.0286 for both models. FIG. 3C, Schematic representation of the experiment designed to test whether Flt1 is required for the talazoparib-resistance phenotype in Brca1-def and Bard1-def breast tumors. Res lines derived from the Brca1-def and Bard1-def models described in FIG. 1 were transduced with either control lentivirus (“Lenti-Con”) or lentivirus encoding gRNA for Flt1 (“Flt1i”) and injected into mice. For the Brca1-def model, randomized mice received either vehicle (“Veh”) or talazoparib (“Tal”) treatment starting at two weeks following Res-tumor-cell injection and were euthanized at four weeks following injection. For the Bard1-def model, randomized mice received treatment at one week following Res-tumor cell injection and were euthanized at three weeks following injection. FIG. 3D, Tumor growth curves for the experiment described in C. For the Brca1-def model, n=6 for Lenti-Con+Veh, n=8 for Lenti-Con+Tal, and n=5 for both Flt1i treatment groups. For the Bard1-def model, n=5 for all groups. Data are presented as mean values±SEM. P values were determined with a one-way ANOVA test, comparing endpoint tumor volumes between the Lenti-Con+Tal and Flt1i+Tal groups. In the Brca1-def model, ** at 4 weeks indicates P=0.0073, and in the Bard1-def model, ** at 3 weeks indicates P=0.0053. FIG. 3E, Representative images of tumors following the treatment regimens described in C. FIG. 3F, Tumors weights from C were plotted after collection at endpoint. For the Brca1-def model, n=6 for Lenti-Con+Veh, n=8 for Lenti-Con+Tal, and n=5 for both Flt1i treatment groups. For the Bard1-def model, n=5 for all groups. Data are presented as mean values±SEM. P values were determined by a two-tailed, unpaired, Mann-Whitney test, comparing endpoint tumor weights between the Lenti-Con+Tal and Flt1i+Tal groups. For the Brca1-def model, ** indicates P=0.0016. For the Bard1-def model, ** indicates P=0.0079. FIG. 3G, Schematic representation of the collection and analysis of patient samples for pFLT1 expression in cancer cells after PARPi treatment. pFLT1 immunostainings were performed on tissue specimens (biopsies/resected material) from 12 patients with breast cancer that were obtained following PARPi treatment and collected at the time of acquired resistance. The immunostained samples were scored by pathologists blinded to the sample details as either pFLT1-low expression (positively stained tissue sections scored between 0 and 1) or pFLT1-high expression (positively stained tissue sections scored above 1 until 4). FIG. 3H, Kaplan-Meier plots for the PFS of patients described in G. Data were analyzed using the log-rank test: χ2=10.26, degrees of freedom (d.f.)=1; P=0.0014, n=11 patients.

FIG. 4A-FIG. 4H—Pharmacological inhibition of FLT1/VEGFR1 sensitizes talazoparib-resistant tumors to talazoprib treatment. FIG. 4A, Schematic representation of the experiment designed to test whether FLT1 inhibition re-sensitizes talazoparib-resistant Brca1-def tumors to talazoparib. Mice were injected with the talazoparib-resistant (“Res”) Brca1-def breast cancer cell line from FIG. 1 and then randomized into the following four treatment groups at two weeks post tumor-cell injection: 1) vehicle (“Veh”), 2) talazoparib (“Tal”), 3) axitinib (“Axi”), and 4) Tal+Axi. Tumor size was measured weekly to track tumor growth, and mice were euthanized for tumor collection at four weeks post tumor-cell injection. FIG. 4B, Tumor growth curves for the mice treated as described in A. n=6 Veh-treated tumors, n=7 Tal-treated tumors, n=5 Axi-treated tumors, and n=5 tumors treated with Tal+Axi. Data are presented as mean values #SEM. P values were determined with a one-way ANOVA test, comparing endpoint tumor volumes between the Tal and Tal+Axi groups. **** at 4 weeks indicates P<0.0001. FIG. 4C, Representative images of tumors from A. FIG. 4D, Tumor weights were plotted after collection at endpoint. n=6 Veh-treated tumors, n=7 Tal-treated tumors, n=5 Axi-treated tumors, and n=5 tumors treated with Tal+Axi. Data are presented as mean values±SEM. P values were determined by a two-tailed, unpaired, Mann-Whitney test, comparing endpoint tumor weights between the Tal and Tal+Axi groups. ** indicates P=0.0051. FIG. 4E, The same methodologies used in A were applied to Res tumors from the Bard1-def model to test whether FLT1 inhibition re-sensitizes talazoparib-resistant Bard1-def tumors to talazoparib. Treatment started at one week post Res tumor-cell injection, and tumors were harvested at three weeks post tumor-cell injection. FIG. 4F, Tumor growth curves for tumors in E. n=5 Veh-treated tumors, n=6 Tal-treated tumors, n=5 Axi-treated tumors, and n=7 tumors treated with Tal+Axi. Data are presented as mean values±SEM. P values were determined with a one-way ANOVA test comparing endpoint tumor volumes between the Tal and Tal+Axi groups. **** at 3 weeks indicates P<0.0001. FIG. 4G, Representative images of tumors from E. FIG. 4H, Tumor weights were plotted after collection at endpoint. n=5 Veh-treated tumors, n=6 Tal-treated tumors, n=5 Axi-treated tumors, and n=7 tumors treated with Tal+Axi. Data are presented as mean values±SEM. P values were determined by a two-tailed, unpaired, Mann-Whitney test comparing endpoint tumor weights between the Tal and Tal+Axi groups. *** indicates P=0.0006.

FIG. 5A-FIG. 5F—The pro-survival AKT pathway is activated in talazoparib-resistant VEGFR1-proficient tumor cells from Brca1-def and Bard1-def breast cancer models. FIG. 5A, Representative images of IHC for pAKT on talazoparib-sensitive (“Sen”) and -resistant (“Res”) tumors from Brca1-def and Bard1-def breast cancer models in FIG. 1. FIG. 5B, Immunostained sections from A were quantified using automated QuPath software to identify positively stained cells. n=5 for Sen and Res groups for both models. Data are presented as mean values±SEM. P values were determined by a two-tailed, unpaired, Mann-Whitney test: * indicates P=0.0159 for both models. FIG. 5C, Representative images of IHC for pAKT IHC on Lenti-Con- and Flt1i-expressing Res tumor sections from both models in FIG. 3C. FIG. 5D, Immunostained sections from C were quantified using automated QuPath software to identify positively stained cells. n=5 for both Lenti-Con+talazoparib (“Tal”) and Flt1i+Tal groups for the Brca1-def and Bard1-def models. Data are presented as mean values SEM. P values were determined by a two-tailed, unpaired, Mann-Whitney test. ** indicates P=0.0079 for the Brca1-def model, and * indicates P=0.0159 for the Bard1-def model. FIG. 5E, Representative images of IHC for pAKT on Tal- and Tal+Axi-treated tumor sections from both models in FIG. 4A/E. FIG. 5F, Immunostained sections from E were quantified using automated QuPath software to identify positively stained cells. For the Brca1-def model, n=4 for tumors from both Tal-treated mice and Tal+Axi-treated mice. For the Bard1-def model, n=5 tumors from both Tal-treated mice and Tal+Axi-treated mice. Data are presented as mean values±SEM. P values were determined by a two-tailed, unpaired, Mann-Whitney test. In the Brca1-def model, * indicates P=0.0286 and in the Bard1-def model, * indicates P=0.0317.

FIG. 6A-FIG. 6J—T-cell-dependent tumor regression induced by the combination of talazoparib (“Tal”) and FLT1 blockade in talazoparib-resistant (“Res”) breast cancer models. FIG. 6A, Representative images of IHC for CD8α on Lenti-Con- and Flt1i-expressing Res tumor sections from the Brca1-def and Bard1-def breast cancer models described in FIG. 3C. FIG. 6B, Immunostained sections from A were quantified using automated QuPath software to identify positively stained cells. n=5 for both Lenti-Con+Tal and Flt1i+Tal tumors from both models. Data are presented as mean values±SEM. P values were determined by a two-tailed, unpaired, Mann-Whitney test. ** indicates P=0.0079 for both models. FIG. 6C, Cell lines derived from Lenti-Con- and Flt1i-expressing Brca1-def and Bard1-def Res tumors (see FIG. 3C) were injected into immunodeficient mice (“T-cell-def mice”). Following Res tumor-cell injection, both Lenti-Con and Flt1i groups were treated with Tal. For the Brca1-def model, treatment started at two weeks post tumor-cell injection, and tumors were collected at four weeks post tumor-cell injection. For the Bard1-def model, treatment started at one-week post tumor cell injection, and tumors were collected at three weeks post tumor-cell injection. Tumor size was measured weekly. In the Brca1-def model, n=5 for both Lenti-Con+Tal and Flt1i+Tal groups. In the Bard1-def model, n=4 for both Lenti-Con+Tal and Flt1i+Tal groups. Data are presented as mean values±SEM. P values were determined by a two-tailed, unpaired, Mann-Whitney test. For the Brca1-def model, * at 2 weeks indicates P=0.0317 and * at 3 weeks indicates P=0.0159; ns: not significant. FIG. 6D, Representative images of tumors from the experiment described in C. FIG. 6E, Tumor weights were plotted after collection at endpoint. In the Brca1-def model, n=5 for both Lenti-Con+Tal and Flt1i+Tal tumors. For the Bard1-def model, n=4 for both Lenti-Con+Tal and Flt1i+Tal tumors. Data are presented as mean values±SEM. P values were determined by a two-tailed, unpaired, Mann-Whitney test. ns: not significant. FIG. 6F, Representative images of IHC for CD8α on Res tumor sections from the mice treated with either Tal or Tal+Axi (described in FIG. 4A/E). FIG. 6G, Immunostained sections from F were quantified using automated QuPath software to identify positively stained cells. n=5 for both Tal and Tal+Axi groups from both models. Data are presented as mean values±SEM. P values were determined by a two-tailed, unpaired, Mann-Whitney test. For the Brca1-def model, ** indicates P=0.0079. For the Bard1-def model, * indicates P=0.0159. FIG. 6H, Brca1-def and Bard1-def Res tumor cells were injected into T-cell-def mice. Mice were then randomized to receive either Tal or Tal+Axi treatments. For the Brca1-def model, treatment started at two weeks post tumor-cell injection and tumors were collected at four weeks post tumor-cell injection. For the Bard1-def model, treatment started at one-week post tumor-cell injection and tumors were collected at three weeks post tumor-cell injection. For the Brca1-def model, n=4 for both Tal and Tal+Axi groups. In the Bard1-def model, n=5 for both Tal and Tal+Axi groups. Data are presented as mean values±SEM. P values were determined by a two-tailed, unpaired, Mann-Whitney test. ns: not significant. FIG. 6I, Representative images of tumors treated with either Tal or Tal+Axi (from H) for both models. FIG. 6J, Tumor weights were plotted after collection at endpoint. For the Brca1-def model, n=4 for both Tal and Tal+Axi groups. For the Bard1-def model, n=5 for both Tal and Tal+Axi groups. Data are presented as mean values±SEM. P values were determined by a two-tailed, unpaired, Mann-Whitney test. ns: not significant.

FIG. 7A-FIG. 7B—High pFLT1 expression in human breast tumors prior to PARPi treatment is associated with shorter PFS on PARPi in breast cancer patients. FIG. 7A, Schematic representation of the analysis of patient samples for pFLT1 expression in cancer cells. pFLT1 immunostainings were performed on tissue specimens (biopsies/resected material) from 10 patients with breast cancer that were obtained prior to PARPi treatment. The immunostained samples were scored by independent pathologists as either pFLT1-low expression (positively stained cells scored between 0 and 1) or pFLT1-high expression (positively stained tissue sections scored above 1 until 4). FIG. 7B, Kaplan-Meier plots for the PFS of patients described in A. Data were analyzed using the log-rank test: χ2=5.574, degrees of freedom (d.f.)=1; P=0.0182; n=10 patients.

FIG. 8A-FIG. 8B—Modest differences observed in vitro for talazoparib sensitivity between talazoparib-sensitive and -resistant Brca1-def and Bard1-def breast cancer cell lines. FIG. 8A-B, In vitro cell viability assay comparing talazoparib sensitivity between talazoparib-sensitive (“Sen”) Brca1-def and Bard1-def breast cancer cell lines to their derived talazoparib-resistant (“Res”) lines. Cell viability values are normalized to the DMSO-treated control and presented as mean values±SEM. P values were determined by a two-tailed, unpaired, Welch's test. For the Brca1-def model, n=3 for each concentration tested, ** at 0.3 nM indicates P=0.0028, * at 1 nM indicates P=0.0104, * at 3 nM indicates P=0.0104, and * at 100 nM indicates P=0.0357. For the Bard1-def model, n=3 for each tested concentration, and * at 3.0 nM indicates P=0.0256.

FIGS. 9A-FIG. 9C—Quantification of angiogenesis and immune-cell composition in Brca1-def and Bard1-def mutant breast tumors by immunostaining analysis. FIGS. 9A-B, Immunostained tumor sections from FIG. 1 were quantified using automated QuPath software to identify positively stained cells. Data are presented as mean values±SEM. P values were determined by a two-tailed, unpaired, Welch's test. n=4 for talazoparib-sensitive (‘Sen”) and -resistant (“Res”) groups from both models. For the Brca1-def model in A, P values are listed in parentheses following the protein analyzed: CD8α (0.0375), CD4 (0.0329), CD11C (0.0061), F4/80 (0.0027), and FOXP3 (0.0007). For the Bard1-def model in B, P values are listed in parentheses following the protein analyzed: CD8α (0.0293), CD4 (0.0186), CD11C (0.0048), F4/80 (0.0016), and S100A9 (0.0182). FIG. 9C, Immunostained sections of Res tumors from both models in FIG. 2I were stained with anti-mouse antibody against CD31 and quantified using automated QuPath software to identify positively stained cells. n=5 for both models. P values were determined by a two-tailed, unpaired, Mann-Whitney test. ** indicates P=0.0079 for the Brca1-def model, and * indicates 0.0159 for the Bard1-def model.

FIG. 10A-FIG. 10G—Talazoparib treatment activates FLT1 signaling in cancer cells. FIG. 10A, Representative images of IHC for FLT1/VEGFR1 on tumor sections from FIG. 1. FIG. 10B, Immunostained sections from A were quantified using automated QuPath software to identify positively stained cells. n=5 for talazoparib-sensitive (“Sen”) tumors, and n=4 for talazoparib-resistant (“Res”) tumors from both Brca1-def and Bard1-def models. Data are presented as mean values±SEM. P values were determined by a two-tailed, unpaired, Mann-Whitney test. * indicates P=0.0159 for both models. FIG. 10C, Representative images of IHC for FLT4/VEGFR3 on tumor sections from FIG. 1. FIG. 10D, Immunostained sections from C were quantified using automated QuPath software to identify positively stained cells. n=4 for Sen and Res tumors for both Brca1-def and Bard1-def models. Data are presented as mean values±SEM. P values were determined by a two-tailed, unpaired, Welch's test. ns: not significant. FIG. 10E-F, Real time quantitative RT-PCR results confirmed successful knockdown of Flt1 expression level in both models. n=6 for Lenti-Con and Flt1i groups for both models. Data are presented as mean values±SEM. P values were determined by a two-tailed, unpaired, Welch's test: *** indicates P<0.0001. FIG. 10G, Kaplan-Meier plots for the PFS of patients described in FIG. 3G. Patients samples were stained with total FLT1. Data were analyzed using the log-rank test: χ2=9.128, degrees of freedom (d.f.)=1; P=0.0025; n=11 patients.

FIG. 11A-FIG. 11B—Body weight analysis of mice treated with vehicle (“Veh”), talazoparib (“Tal”) or axitinib (“Axi”) both individually and in combination. FIG. 11A-B, Body weight, which was used as an indicator for the overall health of the mice tested as described in FIGS. 4A and 4E, remained stable from treatment initiation until endpoint for each experiment. For the Brca1-def model, n=6 Veh-treated tumors, n=7 Tal-treated tumors, n=5 Axi-treated tumors, and n=5 tumors treated with Tal+Axi. For the Bard1-def model, n=5 Veh-treated tumors, n=6 Tal-treated tumors, n=5 Axi-treated tumors, and n=7 tumors treated with Tal+Axi.

FIG. 12A-FIG. 12B—Quantification of pSTAT3 levels in sensitive “Sen” and resistant “Res” Brca1-def and Bard1-def mutant breast tumors by immunostaining analysis. FIG. 12A, Representative images of IHC for pStat3 on tumor sections from mice described in FIG. 1 comparing talazoparib-sensitive (“Sen”) tumors and talazoparib-resistant (“Res”) tumors. FIG. 12B, Immunostained sections from A were quantified using automated QuPath software to identify positively stained cells. In the Brca1-def model, n=5 for talazoparib-sensitive (“Sen”) tumors, and n=4 for talazoparib-resistant (“Res”) tumors. In the Bard1-def model, n=4 for both Sen and Res tumors. Data are presented as mean values±SEM. P values were determined by a two-tailed, unpaired, Mann-Whitney test. ns: not significant.

FIG. 13A-FIG. 13B—Immune changes in the tumor microenvironment following FLT1 blockade and talazoparib treatment. FIG. 13A, Multiple immune markers were analyzed in talazoparib-resistant (“Res”) tumor cells from both Brca1-def and Bard1-def breast cancer models expressing either Lenti-Con or Flt1i and treated with talazoparib (“Tal”) as described in FIG. 3C. n=5 for both groups from each model. Data are presented as mean values±SEM. P values were determined by a two-tailed, unpaired, Mann-Whitney test. For the Brca1-def model, P values are listed in parentheses following the protein analyzed: CD11C (0.0079), CD4 (0.0079), F4/80 (0.0079), FOXP3 (0.0317), and S100A9 (0.0079). For the Bard1-def model, P values are CD11C (0.0317), CD4 (0.0317), F4/80 (0.0079), FOXP3 (0.0079), and S100A9 (0.0079). FIG. 13B, Multiple immune markers were analyzed in Res tumor cells from both Brca1-def and Bard1-def breast cancer models treated with Tal or Tal+axitinib (“Axi”) as described in FIGS. 4A and 4E. n=5 for both groups from each model. Data are presented as mean values±SEM. P values were determined by a two-tailed, unpaired, Mann-Whitney test. For the Brca1-def model, * indicates P=0.0159 for both B220 and S100A9. For the Bard1-def model, ** indicates P=0.0079 for F4/80.

FIG. 14A-FIG. 14B—High FLT1 expression in human tumors prior to PARPi treatment is associated with shorter PFS in breast cancer patients. FIG. 14A, Schematic representation of the collection and analysis of patient samples for FLT1 expression in cancer cells. FLT1 immunostainings were performed on tissue specimens (biopsies/resected material) from 10 patients with breast cancer that were obtained prior to PARPi treatment. The immunostained samples were scored by independent pathologists as either FLT1-low expression (positively stained cells scored between 0 and 1) or FLT1-high expression (positively stained tissue sections scored above 1 until 4). FIG. 14B, Kaplan-Meier plots for the PFS of patients described in A. Data were analyzed using the log-rank test: χ2=8.044, degrees of freedom (d.f.)=1; P=0.0046; n=10 patients.

FIG. 15 is a representation of PARP inhibitor (PARPi) resistance mediated by FLT1, which is a biomarker and therapeutic target for reversing PARPi resistance in BRCA-mutant breast cancer.

FIG. 16A-FIG. 16E—FLT1 (VEGFR1) activation in tumor cells promotes talazoparib resistance in breast cancer models. (FIG. 16A) Representative images of IHC for phosphorylated FLT1 (pFLT1) on sections from the talazoparib-sensitive (“Sen”) and -resistant (“Res”) Brca1-def and Bard1-def breast cancer tumors from mouse models described in FIG. 1. Scale bars, 20 μm. (FIG. 16B) Immunostained sections from (A) were quantified using automated QuPath software to identify positively stained cells. n=4 tumors for both Sen and Res in both models. Data were presented as mean values±SEM. P values were determined by a two-tailed, unpaired, Mann-Whitney test. * indicates P=0.0286 for both models. (FIG. 16C) Schematic representation of the experiment designed to test whether Flt1 is required for the talazoparib-resistance phenotype in Brca1-def and Bard1-def mammary tumors. Res lines derived from the Brca1-def and Bard1-def models described in FIG. 1 were transduced with either control lentivirus (“Lenti-Con”) or lentivirus encoding guide RNA for Flt1 (“Flt1i”) using two independent gRNAs and injected into mice. For the Brca1-def model, randomized mice received either vehicle (“Veh”) or talazoparib (“Tal”) treatment starting at 2 weeks following Res-tumor-cell injection and were euthanized at 4 weeks following injection. For the Bard1-def model, randomized mice received treatment at 1 week following Res-tumor-cell injection and were euthanized at 3 weeks following injection. (FIG. 16D) Tumor growth curves for the experiment described in (C). For the Brca1-def model, n=6 for Lenti-Con+Veh, n=8 for Lenti-Con+Tal, n=5 for Flt1i (gRNA1)+Veh or Tal and Flt1i (gRNA2)+Veh, and n=7 for Flt1i (gRNA2)+Tal treatment groups. For the Bard1-def model, n=5 for Lenti-Con+Veh or Tal, Flt1i (gRNA1)+Veh or Tal, and Flt1i (gRNA2)+Tal, and n=3 for Flt1i (gRNA2)+Veh. Data were presented as mean values±SEM. P values were determined with a one-way ANOVA test, comparing endpoint tumor volumes between the Lenti-Con+Tal and Flt1i (gRNA1 and gRNA2)+Tal groups. For the Brca1-def model at 4 weeks, ** between Lenti-Con+Tal and Flt1i (gRNA1)+Tal indicates P=0.0017 and * between Lenti-Con+Tal and Flt1i (gRNA2)+Tal indicates P<0.0001. For the Bard1-def model at 3 weeks, * between Lenti-Con+Tal and Flt1i (gRNA1)+Tal indicates P=0.0190 and ** between Lenti-Con+Tal and Flt1i (gRNA2)+Tal indicates P=0.0021. (FIG. 16E) Representative images of tumors at endpoint are described in (D). Source data are available online for this figure.

FIG. 17A-FIG. 17L—FLT1 promotes PARPi-resistance in the Brca1-def and Bard1-def breast cancer models. (FIG. 17A) Representative images of IHC for total FLT1 expression in tumor sections from the mice described in FIG. 1. Scale bars, 20 μm. (FIG. 17B) Immunostained sections from (A) were quantified using automated QuPath software to identify positively stained cells. n=5 talazoparib-sensitive (“Sen”) tumors, and n=4 talazoparib-resistant (“Res”) tumors from both Brca1-def and Bard1-def models. Data were presented as mean values±SEM. P values were determined by a two-tailed, unpaired, Mann-Whitney test. * indicates P=0.0159 for both models. (FIG. 17C), Representative images of IHC for FLT4 in tumor sections from FIG. 1. Scale bars, 20 μm. (FIG. 17D) Immunostained sections from (C) were quantified using automated QuPath software to identify positively stained cells. n=4 Sen and Res tumors for both Brca1-def and Bard1-def models. Data were presented as mean values±SEM. P values were determined by a two-tailed, unpaired, Welch's test. ns; not significant. (FIG. 17E) qRT-PCR results of Flt1 repression of the indicated groups in the Brca1-def model for both gRNAs. n=6 (consisting of two independent experiments for each triplicate testing) for both Lenti-Con and Flt1i for gRNA1 and n=3 (one triplicate testing) for both Lenti-Con and Flt1i for gRNA2. Data were presented as mean values±SEM. P values were determined by a two-tailed, unpaired, Welch's test: indicates P<0.0001 for gRNA1 and * indicates 0.0285 for gRNA2. (FIG. 17F) qRT-PCR results of Flt1 repression of the indicated groups in the Bard1-def model. n=6 (two independent triplicate testing) for both Lenti-Con and Flt1i for gRNA1 and n=3 (one triplicate testing) for both Lenti-Con and Flt1i for gRNA2. Data were presented as mean values±SEM. P values were determined by a two-tailed, unpaired, Welch's test: **** indicates P<0.0001 for gRNA1 and * indicates P=0.0273 for gRNA2. (FIG. 17G) qRT-PCR results of Flt1 expression of the indicated groups in the Brca1-def model. n=3 (one triplicate testing) for Flt1i and Flt1i+Flt1 overexpression (“o/e”) groups. Data were presented as mean values±SEM. P values were determined by a two-tailed, unpaired, Welch's test: ** indicates P=0.0016. (FIG. 17H) qRT-PCR results of Flt1 expression of the indicated groups in the Bard1-def model. n=3 (one triplicate testing) for Flt1i and Flt1i+Flt1 o/e groups. Data were presented as mean values±SEM. P values were determined by a two-tailed, unpaired, Welch's test: indicates P=0.0067. (FIG. 17I) Tumor weights from FIG. 3D were plotted at endpoint. For the Brca1-def model, n=6 tumors for Lenti-Con+Veh, n=8 tumors for Lenti-Con+Tal, n=5 tumors for Flt1i (gRNA1)+Veh or Tal and Flt1i (gRNA2)+Veh, and n=7 tumors for Flt1i (gRNA2)+Tal treatment groups. For the Bard1-def model, n=5 tumors for Lenti-Con+Veh or Tal, Flt1i (gRNA1)+Veh or Tal, and Flt1i (gRNA2)+Tal, and n=3 tumors for Flt1i (gRNA2)+Veh. Data were presented as mean values±SEM. P values were determined by a two-tailed, unpaired, Mann-Whitney test, comparing endpoint tumor weights between Lenti-Con+Tal and Flt1i (gRNA1 or gRNA2)+Tal groups. For the Brca1-def model, ** indicates P=0.0016 between Lenti-Con+Tal and Flt1i (gRNA1)+Tal and *** indicates P=0.0003 between Lenti-Con+Tal and Flt1i (gRNA2)+Tal. For the Bard1-def model, ** indicates P=0.0079 between Lenti-Con+Tal and Flt1i (gRNA1 or gRNA2)+Tal. (FIG. 17J) Schematic representation of the experiment designed to test whether Flt1 re-expression rescues talazoparib-resistance in Brca1-def and Bard1-def mammary tumors with Flt1 repression. The generation of Brca1- and Bard1-def cancer cells were described in FIG. 3. To stably re-express Flt1, Applicants transduced Flt1-repressed cells with lentiviral particles carrying Flt1 cDNA. For the Brca1-def model, randomized mice received either vehicle (“Veh”) or talazoparib (“Tal”) treatment starting at 2 weeks after tumor-cell injection and were euthanized at 4 weeks following injection. For the Bard1-def model, randomized mice received treatment at 1 week after tumor-cell injection and were euthanized at 3 weeks following injection. (FIG. 17K) Tumor growth curves comparing Flt1i+Tal and Flt1i-Flt1 o/e+Veh or Tal. For the Brca1-def model, n=7 mice for Flt1i+Tal, n=4 mice for Flt1i-Flt1+o/e+Veh, and n=3 mice for Flt1i-Flt1+o/e+Tal. For the Bard1-def model, n=5 mice for Flt1i+Tal, n=4 mice for Flt1i-Flt1+o/e+Veh, and n=6 mice for Flt1i-Flt1+o/e+Tal. Data were presented as mean values±SEM. P values were determined with a one-way ANOVA test, comparing endpoint tumor volumes between the Flt1i+Tal and Flt1i-Flt1+o/e+Tal groups. For the Brca1-def model, *** at 4 weeks indicates P=0.0001 and for the Bard1-def model, ** at 3 weeks indicates P=0.0074. (FIG. 17L) Tumor weights from K were plotted at endpoint. For the Brca1-def model, n=7 tumors for Flt1i+Tal, n=4 tumors for Flt1i-Flt1+o/e+Veh, and n=3 tumors for Flt1i-Flt1+o/e+Tal. For the Bard1-def model, n=5 tumors for Flt1i+Tal, n=4 tumors for Flt1i-Flt1+o/e+Veh, and n=6 tumors for Flt1i-Flt1+o/e+Tal. Data were presented as mean values±SEM. P values were determined by a two-tailed, unpaired, Mann-Whitney test, comparing endpoint tumor weights between the Flt1i+Tal and Flt1i-Flt1+o/e+Tal groups. For the Brca1-def model, * indicates P=0.0167 and for the Bard1-def model, ** indicates P=0.0043.

FIG. 18A-FIG. 18H—FLT1 activation in Brca1- and Bard1-def breast tumor cells induces pro-survival AKT signaling. (FIG. 18A, FIG. 18B) Immunoblot analysis was performed on lysates from Flt1-expressing (Con) and -deficient (Flt1i), talazoparib-resistant (“Res”) Brca1- and Bard1-def tumor cells, that were treated with 50 ng/ml of mouse PGF protein using antibodies against phosphorylated-AKT at serine 473 (pAKT Ser473), AKT, and β-actin. (FIG. 18C) Representative images of IHC for pAKT Ser473 staining on talazoparib-sensitive (“Sen”) and -Res tumors from the Brca1-def and Bard1-def breast cancer models from FIG. 1. Scale bars, 20 μm. (FIG. 18D) Immunostained sections from (C) were quantified using automated QuPath software to identify positively stained cells. n=5 mice for Sen and Res groups for both models. Data were presented as mean values±SEM. P values were determined by a two-tailed, unpaired, Mann-Whitney test: * indicates P=0.0159 for both models. (FIG. 18E) Representative images of IHC for pAKT Ser473 staining on talazoparib-treated “Tal”, Lenti-Con- and Flt1i-expressing Res tumor sections from both models (see FIG. 3C). Scale bars, 20 μm. (FIG. 18F) Immunostained sections from (E) were quantified using automated QuPath software to identify positively stained cells. n=5 mice for both Lenti-Con+Tal and Flt1i+Tal groups for the Brca1-def and Bard1-def models. Data are presented as mean values±SEM. P values were determined by a two-tailed, unpaired, Mann-Whitney test. ** indicates P=0.0079 for the Brca1-def model, and * indicates P=0.0159 for the Bard1-def model. (FIG. 18G) Representative images of IHC for pAKT Ser473 on Tal-treated and Tal+Axi-treated tumor sections from both models (see FIG. 4A/D). Scale bars, 20 μm. (FIG. 18H) Immunostained sections from (G) were quantified using automated QuPath software to identify positively stained cells. For the Brca1-def model, n=4 tumors from both Tal-treated and Tal+Axi-treated mice. For the Bard1-def model, n=5 tumors from both Tal-treated and Tal+Axi-treated mice. Data were presented as mean values±SEM. P values were determined by a two-tailed, unpaired, Mann-Whitney test. For the Brca1-def model, * indicates P=0.0286 and for the Bard1-def model, * indicates P=0.0317. Source data are available online for this figure.

FIG. 19A-FIG. 19D—Quantitation of phosphorylated STAT3 (pSTAT3) levels in Brca1-def and Bard1-def breast tumor cells and tumor tissue sections. (FIG. 19A, FIG. 19B) Immunoblot analysis was performed using antibodies against pSTAT3, STAT3 and β-actin (loading control) using lysates from Flt1-expressing (Con) and -deficient (Flt1i), talazoparib-resistant (“Res”) Brca1 and Bard1-def tumor cells, treated with 50 ng/ml of mouse PGF protein that were used in FIG. 5A, B. (FIG. 19C) Representative images of IHC for pSTAT3 staining in tumor sections from the mice described in FIG. 1 comparing talazoparib-sensitive (“Sen”) tumors to talazoparib-resistant (“Res”) tumors. Scale bars, 20 μm. (FIG. 19D) Immunostained sections from (C) were quantified using automated QuPath software to identify positively stained cells. For the Brca1-def model, n=4 tumors for both Sen and Res. For the Bard1-def model, n=5 Sen tumors, and n=4 Res tumors. Data were presented as mean values±SEM. P values were determined by a two-tailed, unpaired, Mann-Whitney test. ns; not significant.

FIG. 20A-FIG. 20C—High pFLT1 expression in human breast tumors prior to PARPi treatment is associated with shorter progression-free survival on PARPi in patients with breast cancer. (FIG. 20A) Schematic representation of the workflow for the pathological evaluation of pFLT1 expression in tumor cells from breast cancer patients before PARPi treatment. pFLT1 immunostainings were performed on tissue specimens (biopsies/resected material) from ten patients with breast cancer and were obtained prior to PARPi treatment. The immunostained samples were scored by a pathologist, who was blinded to the sample details, as either pFLT1-high or -low expression. (FIG. 20B) Representative IHC images of high or low levels of pFLT1 expression on tumor tissue samples from patients with breast cancers. Scale bars, 20 μm. (FIG. 20C) Kaplan-Meier plots for the PFS of patients described in (A). Data were analyzed using the log-rank test: χ2=6.325, degrees of freedom (d.f.)=1; P=0.012; n=10 patients. Source data are available online for this figure.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The present disclosure provides for methods of treating PARP inhibitor (PARPi) resistance in a cancer where the standard of care is PARPi or a cancer initially responsive to PARPi by blockade of FLT1 (VEGFR1) resulting in suppression of AKT activation, increase in tumor infiltration of CD8⁺ T cells, and dramatic regression of PARPi-resistant tumors in a T-cell-dependent manner.

### 1. Overview

The development of PARPi resistance is a major clinical challenge for the successful treatment of BRCA1-mutant breast cancer⁴⁸. PARPi treatment elicits 60% response rates and increased progression-free survival compared to chemotherapy, but it fails to improve overall survival due to the onset of drug resistance^{17,18,49}. Overcoming PARPi resistance, therefore, is expected to improve the effectiveness of PARPi treatment and to extend the survival of breast cancer patients. Here, Applicants present a FLT1-driven, therapeutically targetable pathway that can be used to stratify and treat BRCA1-mutant breast cancer patients who become resistant to PARPi treatment.

Applicants present newly generated in-vivo PARPi-treatment-response-and-recurrence models using Brca1-def and Bard1-def orthotopic allograft models that recapitulate the phases of PARPi-therapy response and treatment failure observed in BRCA1-mutant breast cancer patients. In contrast to the well-established on-target genetic PARPi-resistance mechanisms, Applicants demonstrate how an adaptive mechanism driven by PIGF-FLT1-AKT signaling protects Brca1- and Bard1-deficient breast cancer cells from PARPi-induced cell death. The experimental studies show that PIGF expression increases locally in the tumor milieu upon PARPi treatment. Thereafter, a minor subpopulation of Brca1- and Bard1-deficient cancer cells that expresses the PIGF receptor FLT1 prior to PARPi treatment becomes enriched in the PARPi-resistant tumors. The FLT1 pathway therefore represents a vulnerability that can be targeted to overcome PARPi resistance. The preclinical studies offer a new biomarker-guided combination treatment option that includes a PARPi (talazoparib) and a VEGFR inhibitor (axitinib) to specifically target PARPi-resistant breast cancers that express FLT1.

The function of FLT1 varies by cell type and cellular context. Flt1 is normally expressed in endothelial cells, immune cells, and hematopoietic stem cells (HSCs)³⁸. In the context of angiogenesis, Flt1 is essential for the organization of embryonic vasculature but not for endothelial-cell differentiation^{50,51} FLT1 also functions as a signaling receptor in myeloid cells and HSCs, by promoting their chemotaxis and migration in response to VEGF and/or PIGF⁵²⁻⁵⁵. In the context of cancer, the removal of Flt1⁺ bone-marrow-derived hematopoietic progenitor cells reduces pre-metastatic clusters and prevents tumor metastasis in mice⁵⁶. In addition, FLT1 signaling in macrophages activates an inflammatory response and promotes breast cancer metastasis⁵⁷. In contrast to these studies, Applicants show that FLT1 is expressed in the breast cancer cells themselves, and that FLT1 mediates PARPi resistance in breast cancer through a combination of cell-intrinsic and -extrinsic pathways.

FLT1 expression has been previously reported in a subset of cancer cells where it promotes tumor growth by activating mitogenic pathways^{58,59 60-63}. In line with these observations, the efficacy of anti-PIGF antibodies strongly correlates with the expression of tumor-derived FLT1 but not with the inhibition of angiogenesis. This is consistent with the finding that the expression and activation of FLT1 in the cancer cells from the Brca1- and Bard1-deficient breast cancer models represents a key determinant of PARPi resistance in vivo. However, one point of distinction between the current study and the published literature is that Flt1-proficient and -deficient cancer cells show comparable growth kinetics in the absence of PARPi treatment in the tumor models. The difference in tumor growth only becomes apparent after PARPi treatment when Flt1-deficient cancer cells are eliminated, and Flt1-proficient cells persist. Applicants hypothesized that this phenotype arose due to increased PIGF expression in tumors following PARPi treatment, which facilitates pro-survival signaling in FLT1-proficient (but not-deficient) cancer cells. In normal physiology, genetically and pharmacologically inactivating PARP has been shown to reduce angiogenesis in response to growth factors (such as VEGF or PIGF⁶⁴); however, in the pathological context of tumors, Applicants found that it causes an increase in CD31⁺ blood vessels, VEGFR2 expression, and angiogenesis in PARPi-resistant tumors compared to PARPi-sensitive ones. In addition, Vegfr2 inhibition in combination with talazoparib only modestly reduced tumor growth in the Brca1-def and Bard1-def models, whereas Flt1 inhibition in combination with talazoparib resulted in a more prominent tumor-regression phenotype. These data suggest that contrary to ovarian cancer models⁶⁵, VEGFR2-induced angiogenesis does not appear to be a critical component of the growth and survival of PARPi-resistant breast cancer models. Specifically, Bizzaro, et al. relates to the treatment of patient-derived ovarian cancer xenografts (i.e., immunocompromised nude mice) where the combination showed anti-tumour activity, regardless of the homologous recombination repair (HRR) mutational status, tumour cell expression of receptor tyrosine kinases targeted by cediranib, and expression of genes associated with DNA repair machinery (see, e.g., Bizzaro F, Fuso Nerini I, Taylor M A, et al. VEGF pathway inhibition potentiates PARP inhibitor efficacy in ovarian cancer independent of BRCA status. J Hematol Oncol. 2021; 14 (1): 186). In other words, VEGFR1 inhibition as disclosed herein is relevant when the cancer has a defect in HRR, is resistant to PARPi, and/or has increased expression of PIGF and VEGFR1 on the cancer cells.

The data herein implicates a previously unexplored cancer-cell-specific role for the FLT1 pathway in preventing a cytotoxic immune response in PARPi-resistant tumors. Applicants observed a reduction in CD8⁺ T-cell number specifically in the PARPi-resistant tumors from both Brca1- and Bard1-deficient models. Inhibition of FLT1 in PARPi-resistant cancer cells led to increased CD8⁺ T-cell infiltration and tumor regression, which was then reversed in T-cell-deficient mice. The findings therefore suggest that FLT1 inhibition in cancer cells has two effects. First, it interrupts pro-survival PIGF-FLT1-AKT signaling in cancer cells, and second, it increases T-cell infiltration and the immune response, possibly by altering the secretome. It is likely that both mechanisms help sensitize the PARPi-resistant tumors to PARPi treatment. It is currently unclear how FLT1 inhibition in cancer cells alters the secretome and impacts T cells, so future studies will be needed to determine whether the suppression of T-cell cytotoxicity results from decreased chemotaxis, proliferation, or immunosuppression, or through interactions with other immunosuppressive cells in the tumor microenvironment. Although the PIGF-FLT1 axis in tumor cells has not been investigated in the context of adaptive immunity, PIGF secretion has been linked to immunosuppression. For instance, PIGF can induce dendritic-cell dysfunction and suppression of naïve CD4⁺ T-cell proliferation, thereby skewing T-cell responses toward Th2⁵⁸. PIGF can also immunosuppress CD8⁺ T cells by macrophage polarization¹⁹. It is possible that increased PIGF expression following PARPi treatment reprograms immune cells to maintain an immunosuppressive tumor microenvironment, which can be further exacerbated by FLT1 signaling in tumor cells.

In addition to BRCA-mutant breast cancer, PARPi treatment is also approved for the treatment of ovarian, pancreatic, and prostate cancer. Interestingly, cancer-cell expression of FLT1 expression has been reported in ovarian, pancreatic, and prostate cancer cells^{19,61,66} suggesting that FLT1 signaling could potentially drive PARPi resistance in these cancers as well. PARPi's have been approved to treat ovarian cancer patients with and without BRCA mutations and/or deficiencies in HR, and combined treatment with the PARPi olaparib and the VEGFA-selective blocker bevacizumab is FDA-approved for ovarian cancer as maintenance therapy.

Importantly, the combination olaparib and the pan-VEGFR inhibitor cediranib led to a significantly longer median progression-free survival (PFS, 16.5 vs. 8.2 months, hazard ratio of 0.50, p=0.007) compared to olaparib alone in a randomized Phase II study of relapsed high-grade ovarian cancer patients. In a randomized 1:1:1 Phase III study in ovarian cancer patients comparing 1) cediranib-plus-olaparib, 2) olaparib, and 3) platinum chemotherapy (the standard of care), the treatment with cediranib-plus-olaparib showed similar clinical activity to platinum in the BRCA1/2-mutation-positive group. However, in the BRCA-wild-type group, cediranib-plus-olaparib significantly prolonged PFS compared to olaparib alone in both trials, suggesting that alternative modes of action for this combination treatment exist beyond HR-dependent synthetic lethality. From the clinical trial data, it will be interesting to retrospectively determine whether PARPi treatment activates PIGF-FLT1 signaling in human ovarian cancer cells (similar to breast cancer models), which, when effectively blocked by cediranib-plus-olaparib, translates to clinical responses independent of BRCA status.

The clinical sample analysis in the present study showed a strong association between FLT1 activity at pre-treatment and PARPi resistance in breast cancer patients with mutations in BRCA1, BRCA2, or PALB2. Although the sample size is small, these findings lay the foundation for future biomarker studies to test whether FLT1 expression in pre-treatment biopsies can be used to stratify breast cancer patients at high risk for rapidly acquiring resistance to PARPis. Importantly, these patients might clinically benefit from combined inhibition of PARP and FLT1. The study also highlights the need for testing non-genetic markers of PARPi resistance in patient samples by immunohistochemical staining for activated FLT1 in tumor tissues pre- and post-treatment, which would be missed by DNA sequencing analysis. Contrary to the accepted notion regarding the role of angiogenesis in PARPi resistance⁶⁷, the findings suggest that blocking angiogenic pathways driven by VEGFA or VEGFR2 alone might have only minimal ability to reverse PARPi resistance. Instead, a pan-VEGFR inhibitor, such as axitinib, is likely to be more effective at resensitizing PARPi-resistant breast cancers to PARPis. Since axitinib is already FDA-approved for metastatic renal-cell carcinoma⁴² and is currently being tested in combination with talazoparib in a Phase Ib/II clinical trial (NCT04337970), the findings provide the rationale for testing this combination treatment in breast cancer patients with mutations in BRCA1, BRCA2, or PALB2. Of clinical relevance, a recent Phase Ib clinical trial (NCT04693468) known as TalaCom is testing the combination of talazoparib and axitinib across cancer types. It will be important to retrospectively analyze whether talazoparib-plus-axitinib is more effective at delaying cancer progression in patients with high levels of FLT1 expression. If both PIGF and FLT1 signaling are increased in the PARPi-resistant human tumors across cancer types, future studies may show that anti-PIGF antibodies offer another opportunity for therapeutic intervention in addition to VEGFR inhibitors. This is particularly encouraging with renewed interest in TB-403, a monoclonal PIGF-blocking antibody^{68,69}, which has shown success in a Phase I clinical trial for medulloblastoma patients⁷⁰.

The treatment and diagnostic methods described herein may also include detection of PIGF, VEGFR1, and activated AKT. Specific implementations may include detection of biomarkers to monitor treatment and to detect resistance. In example embodiments, upon detection of biomarkers a treatment is administered targeting PIGF, VEGFR1, and/or activated AKT. In other example embodiments, subjects resistant to PARPi are administered a treatment targeting PIGF, VEGFR1, and/or activated AKT. The various implementations may use conventional procedures known to those of ordinary skill in the art as added to and improved upon through the procedures described here.

### 2. Terminology and Definitions

In describing PARP resistance cancer treatment implementations, the following terminology will be used in accordance with the definitions and explanations set out below. Notwithstanding, other terminology, definitions, and explanations may be found throughout this document as well.

As used herein, “FLT1” and “VEGFR1” are used interchangeably and refer to vascular endothelial growth factor receptor 1, which is a protein that in humans is encoded by the FLT1 gene. VEGFR1 is also referred to as FLT1, FLT, FLT-1, VEGFR-1, fms related tyrosine kinase 1, vascular endothelial growth factor receptor 1, and fms related receptor tyrosine kinase 1. VEGFR1 binds VEGFA, VEGFB and placental growth factor (PIGF). The expression of FLT1 and its two ligands, PIGF and VEGFB, is increased in various tumours, which correlates with disease progression and can predict poor prognosis, metastasis and recurrent disease in humans. Representative human sequences for VEGFR1 include: NM_001159920, NM_001160030, NM_001160031, NM_002019, NP_001153392, NP_001153502, NP_001153503, and NP_002010.

As used herein, “PIGF” and “PGF” are used interchangeably herein and refer to placental growth factor. Placental growth factor (PIGF) is a protein that in humans is encoded by the PGF gene. Placental growth factor may also be referred to as D12S1900, PGFL, PLGF, PIGF-2, SHGC-10760, and PIGF. Placental growth factor (PGF) is a member of the VEGF (vascular endothelial growth factor) sub-family, a key molecule in angiogenesis and vasculogenesis. Representative human sequences for PGF include: NM_002632, NM_001207012, NM_001293643, NP_001193941, NP_001280572, and NP_002623.

As used herein, “AKT” refers to the collective name of a set of three serine/threonine-specific protein kinases that play key roles in multiple cellular processes such as glucose metabolism, apoptosis, cell proliferation, transcription, and cell migration. AKT is a serine/threonine kinase, also known as protein kinase B or PKB. AKT1 is also referred to as AKT, CWS6, PKB, PKB-ALPHA, PRKBA, RAC, RAC-ALPHA, and AKT serine/threonine kinase 1. AKT2 is also referred to as v-akt murine thymoma viral oncogene homolog 2, HIHGHH, PKBB, PKBBETA, PRKBB, RAC-BETA, and AKT serine/threonine kinase 2. AKT3 is also referred to as MPPH, MPPH2, PKB-GAMMA, PKBG, PRKBG, RAC-PK-gamma, RAC-gamma, STK-2, and AKT serine/threonine kinase 3. AKT1 and the related AKT2 are activated by platelet-derived growth factor. The activation is rapid and specific, and it is abrogated by mutations in the pleckstrin homology domain of AKT1. It was shown that the activation occurs through phosphatidylinositol 3-kinase. In the developing nervous system AKT is a critical mediator of growth factor-induced neuronal survival. Survival factors can suppress apoptosis in a transcription-independent manner by activating the serine/threonine kinase AKT1, which then phosphorylates and inactivates components of the apoptotic machinery.

As used herein, “PARP” refers poly(ADP-ribose) polymerase, which is a family of proteins involved in a number of cellular processes such as DNA repair, genomic stability, and programmed cell death. The PARP family comprises 17 members (10 putative). They vary greatly in structure and function within the cell. PARP1, PARP2, VPARP (PARP4), Tankyrase-1 and -2 (PARP-5a or TNKS, and PARP-5b or TNKS2) have a confirmed PARP activity. Others include PARP3, PARP6, TIPARP (or “PARP7”), PARP8, PARP9, PARP10, PARP11, PARP12, PARP14, PARP15, and PARP16.

As used herein, “BRCA1” refers to Breast cancer type 1 susceptibility protein, which is a protein that in humans is encoded by the BRCA1 gene (also known as breast cancer 1, early onset, BRCAI, BRCC1, BROVCA1, IRIS, PNCA4, PPP1R53, PSCP, RNF53, FANCS, breast cancer 1, DNA repair associated, BRCA1 DNA repair associated). Orthologs are common in other vertebrate species, whereas invertebrate genomes may encode a more distantly related gene. BRCA1 is a human tumor suppressor gene (also known as a caretaker gene) and is responsible for repairing DNA. BRCA1 and BRCA2 are unrelated proteins, but both are normally expressed in the cells of breast and other tissue, where they help repair damaged DNA, or destroy cells if DNA cannot be repaired. Representative human sequences for BRCA1 include: NM_007294, NM_007295, NM_007296, NM_007297, NM_007298, NP_009225, NP_009228, NP_009229, NP_009230, and NP_009231.

As used herein, “BRCA2” refers to breast cancer type 2 susceptibility protein, which is a human tumor suppressor gene responsible for repairing DNA (also, known as, BRCC2, BROVCA2, FACD, FAD, FAD1, FANCD, FANCD1, GLM3, PNCA2, XRCC11, breast cancer 2, DNA repair associated, breast cancer 2, early onset, and BRCA2 DNA repair associated). Representative human sequences for BRCA2 include: NM_000059 and NP 000050.

As used herein, “triple negative breast cancer” (TNBC) is a term used in its broadest sense and may refer to any breast cancer that either lacks or shows low levels of estrogen receptor (ER), progesterone receptor (PR) and human epidermal growth factor receptor 2 (HER2) overexpression and/or gene amplification (i.e., the tumor is negative on all three tests giving the name triple-negative). Triple-negative is sometimes used as a surrogate term for basal-like.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

For example, in methods for treating cancer in a subject, an effective amount of a combination of agents is any amount that provides an anti-cancer effect, such as reduces or prevents proliferation of a cancer cell or makes a cancer cell responsive to an immunotherapy, a chemotherapy, or targeted cancer drug.

### 3. Methods of Treatment

In example embodiments, the present invention provides for one or more therapeutic agents to treat cancer. In example embodiments, cancer is treated by administering to a subject a PARP inhibitor and a therapeutic for inhibiting VEGFR1 activity. In example embodiments, a PARP inhibitor is administered and upon resistance a VEGFR1 inhibitor is administered. In example embodiments, the PARP inhibitor and VEGFR1 inhibitor are co-administered, administered simultaneously, or administered sequentially. In example embodiments, a VEGFR1 inhibitor is administered before PARP resistance is detected.

In example embodiments, the methods disclosed herein are applicable to any cancer deficient in homologous recombination (HR)-mediated repair of DNA breaks. In example embodiments, the methods disclosed herein are applicable to any BRCA-mutant cancers. In example embodiments, the methods disclosed herein are applicable to any cancer sensitive to PARPi. In example embodiments, the methods disclosed herein are applicable to any cancers having increased expression of VEGFR1 and/or PIGF. Non-limiting examples of cancers that can be treated according to the present invention include breast cancer, ovarian, pancreatic, and prostate cancer.

### VEGFR1 Inhibitors

In example embodiments, a VEGFR1/Flt1 inhibitor is administered to a subject in need thereof. A non-limiting VEGFR1/Flt1 inhibitor is Axitinib (brand name Inlyta). Axitinib is a drug approved 1) for advanced renal cell carcinoma (RCC) after the failure of one prior systemic therapy (2012), and 2) as a first-line treatment for patients with advanced RCC in combination with pembrolizumab (a PD-1 inhibitor) or avelumab (a PD-L1 inhibitor) (each in 2019). Other non-limiting VEGFR inhibitors include pazopanib, sunitinib, bevacizumab, sorafenib, cabozantinib, regorafenib, lenvatinib, ponatinib, cabozantinib, ziv-aflibercept, fruquintinib, tivozanib, ramucirumab, and vandetanib.

### PARP Inhibitors

In example embodiments, a PARP inhibitor is administered to a subject in need thereof. PARP inhibitors are a group of pharmacological inhibitors of the enzyme poly ADP ribose polymerase (PARP). A non-limiting PARP inhibitor is Talazoparib (brand name Talzenna). Talazoparib is a drug approved for the treatment of patients with deleterious or suspected deleterious germline BRCA-mutated (gBRCAm), HER2-negative locally advanced or metastatic breast cancer (2018). Other non-limiting PARP inhibitors include Olaparib, Rucaparib, Niraparib, Veliparib, Pamiparib (BGB-290), CEP 9722, E7016, and 3-Aminobenzamide.

### PIGF Inhibitors

In example embodiments, a PIGF inhibitor is administered to a subject in need thereof. In example embodiments, a PIGF inhibitor is a therapeutic anti-PIGF antibody (see, e.g., Fischer C, Jonckx B, Mazzone M, et al. Anti-PIGF inhibits growth of VEGF (R)-inhibitor-resistant tumors without affecting healthy vessels. Cell. 2007; 131 (3): 463-475.)

### AKT Inhibitors

In example embodiments, AKT activation mediates resistance to PARP inhibitors. In example embodiments, inhibition of AKT signaling is used to reverse or prevent PARPi resistance. In example embodiments, an AKT inhibitor, such as, for example, one or a combination of two or more AKT inhibitors disclosed in claim 8 of WO2017070395 is administered. The term “AKT inhibitor,” “AKTI,” or “AKTi” can be used interchangeably and refers to any molecule (e.g., AKT antagonist), including, but not limited to a small molecule, a polynucleotide (e.g., DNA or RNA), or a polypeptide (e.g., an antibody or an antigen-binding portion thereof), capable of blocking, reducing, or inhibiting the activity of AKT. AKT is a serine/threonine kinase, also known as protein kinase B or PKB. An AKT inhibitor can act directly on AKT, e.g., by binding AKT, or it can act indirectly, e.g., by interfering with the interaction between AKT and a binding partner or by inhibiting the activity of another member of the PI3K-AKT-mTOR pathway. Non-limiting AKT inhibitors include A6730, B2311, 124018, GSK2110183 (afuresertib), Perifosine (KRX-0401), GDC-0068 (ipatasertib), RX-0201, VQD-002, LY294002, A-443654, A-674563, Akti-1, Akti-2, Akti-1/2, AR-42, API-59CJ-OMe, ATI-13148, AZD-5363, erucylphosphocholine, GSK-2141795 (GSK795), KP372-1, L-418, L-71-101, PBI-05204, PIA5, PX-316, SR13668, triciribine, GSK 690693 (CAS #937174-76-0), FPA 124 (CAS #902779-59-3), Miltefosine, PHT-427 (CAS #1 191951-57-1), 10-DEBC hydrochloride, Akt inhibitor III, Akt inhibitor VIII, MK-2206 dihydrochloride (CAS #1032350-13-2), SC79, AT7867 (CAS #857531-00-1), CCT128930 (CAS #885499-61-6), A-674563 (CAS #552325-73-2), AGL 2263, AS-041 164 (5-benzo[1,3]dioxol-5-ylmethylene-thiazolidine-2,4-dione), BML-257 (CAS #32387-96-5), XL-418, CAS #612847-09-3, CAS #98510-80-6, H-89 (CAS #127243-85-0), OXY-1 1 1 A, 3-[1-[[4-(7-phenyl-3H-imidazo[4,5-g]quinoxalin-6-yl)phenyl]methyl]piperidin-4-yl]-1H-benzimidazol-2-one, N,N-dimethyl-1-[4-(6-phenyl-1H-imidazo[4,5-g]quinoxalin-7-yl)phenyl]metha-namine, and 1-{1-[4-(3-phenylbenzo[g]quinoxalin-2-yl)benzyl]piperidin-4-yl}-1,-3-dihydro-2H-benzimidazol-2-one.

### Checkpoint Blockade Therapy

In example embodiments, cytotoxic T cells mediate resistance to PARP inhibitors. In example embodiments, inhibition of VEGFR1 increases CD8+ T cell infiltration. In example embodiments, a CD8+ T cell cytotoxic immune response can be enhanced by checkpoint blockade therapy (CPB). In example embodiments, resistance to PARP inhibitors is reversed resulting in increased CD8+ T cell infiltration and CPB therapy further enhances the cytotoxic CD8+ T cell response.

As used herein, checkpoint blockade or checkpoint inhibitor therapy refers to a form of cancer immunotherapy. The therapy targets immune checkpoints, key regulators of the immune system that when stimulated can dampen the immune response to an immunologic stimulus. Some cancers can protect themselves from attack by stimulating immune checkpoint targets. Antibodies that block the activity of checkpoint receptors, including CTLA-4, PD-1, Tim-3, Lag-3, and TIGIT, either alone or in combination, have been associated with improved effector CD8⁺ T cell responses in multiple pre-clinical cancer models (Johnston et al., 2014. The immunoreceptor TIGIT regulates antitumor and antiviral CD8(+) T cell effector function. Cancer cell 26, 923-937; Ngiow et al., 2011. Anti-TIM3 antibody promotes T cell IFN-gamma-mediated antitumor immunity and suppresses established tumors. Cancer research 71, 3540-3551; Sakuishi et al., 2010. Targeting Tim-3 and PD-1 pathways to reverse T cell exhaustion and restore anti-tumor immunity. The Journal of experimental medicine 207, 2187-2194; and Woo et al., 2012. Immune inhibitory molecules LAG-3 and PD-1 synergistically regulate T-cell function to promote tumoral immune escape. Cancer research 72, 917-927). Similarly, blockade of CTLA-4 and PD-1 in patients (Brahmer et al., 2012. Safety and activity of anti-PD-L1 antibody in patients with advanced cancer. The New England journal of medicine 366, 2455-2465; Hodi et al., 2010. Improved survival with ipilimumab in patients with metastatic melanoma. The New England journal of medicine 363, 711-723; Schadendorf et al., 2015. Pooled Analysis of Long-Term Survival Data From Phase II and Phase III Trials of Ipilimumab in Unresectable or Metastatic Melanoma. Journal of clinical oncology: official journal of the American Society of Clinical Oncology 33, 1889-1894; Topalian et al., 2012. Safety, activity, and immune correlates of anti-PD-1 antibody in cancer. The New England journal of medicine 366, 2443-2454; and Wolchok et al., 2017. Overall Survival with Combined Nivolumab and Ipilimumab in Advanced Melanoma. The New England journal of medicine 377, 1345-1356) has shown increased frequencies of proliferating T cells, often with specificity for tumor antigens, as well as increased CD8⁺ T cell effector function (Ayers et al., 2017. IFN-gamma-related mRNA profile predicts clinical response to PD-1 blockade. The Journal of clinical investigation 127, 2930-2940; Das et al., 2015. Combination therapy with anti-CTLA-4 and anti-PD-1 leads to distinct immunologic changes in vivo. Journal of immunology 194, 950-959; Gubin et al., 2014. Checkpoint blockade cancer immunotherapy targets tumour-specific mutant antigens. Nature 515, 577-581; Huang et al., 2017. T-cell invigoration to tumour burden ratio associated with anti-PD-1 response. Nature 545, 60-65; Kamphorst et al., 2017. Proliferation of PD-1+CD8 T cells in peripheral blood after PD-1-targeted therapy in lung cancer patients. Proceedings of the National Academy of Sciences of the United States of America 114, 4993-4998; Kvistborg et al., 2014. Anti-CTLA-4 therapy broadens the melanoma-reactive CD8⁺ T cell response. Science translational medicine 6, 254ra128; van Rooij et al., 2013. Tumor exome analysis reveals neoantigen-specific T-cell reactivity in an ipilimumab-responsive melanoma. Journal of clinical oncology: official journal of the American Society of Clinical Oncology 31, e439-442; and Yuan et al., 2008. CTLA-4 blockade enhances polyfunctional NY-ESO-1 specific T cell responses in metastatic melanoma patients with clinical benefit. Proceedings of the National Academy of Sciences of the United States of America 105, 20410-20415). Accordingly, the success of checkpoint receptor blockade has been attributed to the binding of blocking antibodies to checkpoint receptors expressed on dysfunctional CD8⁺ T cells and restoring effector function in these cells. The check point blockade therapy may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Anti-PD1 antibodies are disclosed in U.S. Pat. No. 8,735,553. Antibodies to LAG-3 are disclosed in U.S. Pat. No. 9,132,281. Anti-CTLA4 antibodies are disclosed in U.S. Pat. Nos. 9,327,014; 9,320,811; and 9,062,111. Specific check point inhibitors include, but are not limited to anti-CTLA4 antibodies (e.g., Ipilimumab and Tremelimumab), anti-PD-1 antibodies (e.g., Nivolumab, Pembrolizumab, Dostarlimab), and anti-PD-L1 antibodies (e.g., Atezolizumab).

### Small Molecules

In example embodiments, a method of treating cancer comprises administering to a subject in need thereof one or more small molecule modulators that decrease the expression or activity of VEGFR1 signaling. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In example embodiments, the small molecule may act as an antagonist or agonist.

### Degrader Molecules

In example embodiments, inhibiting VEGFR1/Flt1 comprises administering one or more proteolysis targeting chimeras (PROTAC) or degraders. One type of small molecule applicable to the present invention is a degrader molecule (see, e.g., Ding, et al., Emerging New Concepts of Degrader Technologies, Trends Pharmacol Sci. 2020 July; 41 (7): 464-474). The terms “degrader” and “degrader molecule” refer to all compounds capable of specifically targeting a protein for degradation (e.g., ATTEC, AUTAC, LYTAC, or PROTAC, reviewed in Ding, et al. 2020). PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57:107-123; and Lai et al., Modular PROTAC Design for the Degradation of Oncogenic BCR-ABL Angew Chem Int Ed Engl. 2016 Jan. 11; 55 (2): 807-810). In example embodiments, LYTACs are particularly advantageous for cell surface proteins.

More specifically, PROTACs, or Proteolysis Targeting Chimeras, are bifunctional small molecules that induce the degradation of a target protein by targeting it to the ubiquitin-proteasome system (Gilbertson B, Subbarao K. A new route to vaccines using PROTACs. Nat Biotechnol. 2022; 40 (9): 1328-1329). They typically consist of two covalently linked moieties: one that binds to the protein of interest and another that binds to a cytosolic E3 ubiquitin ligase, such as von Hippel-Lindau or cereblon (Crunkhorn S. Developing antibody-based PROTACs. Nat Rev Drug Discov. 2022; 21 (11): 795). By forming a ternary complex with the target protein and the E3 ligase, PROTACs facilitate the ubiquitination and subsequent degradation of the target protein. Id. This therapeutic strategy has gained significant interest in drug development as it enables the targeting of previously undruggable proteins, offering new possibilities for the treatment of various diseases, including cancer (Zografou-Barredo N A, Hallatt A J, Goujon-Ricci J, Cano C. A beginner's guide to current synthetic linker strategies towards VHL-recruiting PROTACs. Bioorg Med Chem. 2023; 88-89:117334; and Gao H, Sun X, Rao Y. PROTAC Technology: Opportunities and Challenges. ACS Med Chem Lett. 2020; 11 (3): 237-240). A number of known PROTAC may be found in the PROTAC-DB database found at cadd.zju.edu.cn/protacdb/about. (See also, Weng et al. (2020). PROTAC-DB: An online database of protacs. Nucleic Acids Research, 49 (D1). doi.org/10.1093/nar/gkaa807) or any others known in literature. For example, TL12-186 can be used to degrade FLT1 (see, e.g., Donovan K A, Ferguson F M, Bushman J W, et al. Mapping the Degradable Kinome Provides a Resource for Expedited Degrader Development. Cell. 2020; 183 (6): 1714-1731.e10).

### Other Bifunctional Molecules

In addition to PROTACS, other bi-functional molecules have been developed that may be used in the context of the present invention. In general, the E3 ligase binding portion is replaced with a binder of an enzyme capable of introducing a post-translational modification (PTM). For example, binders may be selected that bind to deubiquitinases kinases, phosphatases, acetylases, de-acetylases, methylases and demethylases. In embodiments, the enzyme is a kinase, a phosphatase, transferase, glycosyltransferase, ligase, histone acetylases (HATs), or histone deacetylases (HDACs), hydroxylase, a Glutamine Synthetase Adenyl Transferases (GSATase), enzymes catalyzing hydroxylation of protein residues, oxygenase, or sulfotransferase. As with PROTACS, theses binders are then linked to a small molecular binding of the target protein to be modified via linker. The type of bifunctional molecule used will depend on the target gene and whether a particular PTM activates/stabilizes or deactivates/de-stabilizes/degrades that particular target gene. For example, if the goal is to increase activity of the target gene product and phosphorylation is necessary to activate the gene product, then a kinase would be selected. Conversely, if the goal is to increase activity of the target gene product and de-phosphorylation is necessary to activate the product, then a phosphatase would be selected and so forth.

There are several resources available to search for known binders of kinases. One resource is the Published Kinase Inhibitor Set (PKIS), which is a set of 367 small-molecule ATP-competitive kinase inhibitors that was made freely available to expand research in this field. Elkins, J., Fedele, V., Szklarz, M. et al. Comprehensive characterization of the Published Kinase Inhibitor Set. Nat Biotechnol 34, 95-103 (2016). Another resource is the use of databases such as NCI, NPD, and MLSMR, which are frequently used in the virtual screening of kinase inhibitors. Singh, N., Sun, H., Chaudhury, S. et al. A physicochemical descriptor-based scoring scheme for effective and rapid filtering of kinase-like chemical space. J Cheminform 4, 4 (2012). Computational approaches have also been developed to predict molecular targets for small-molecule drugs. R. Cao, Y. Wang, ChemMedChem 2016, 11, 1352.

Phosphorylation-inducing chimeric molecules, also known as Phosphorylation-Inducing Chimeric Small molecules (PHICS), are a new class of small molecules designed to induce phosphorylation, a process that alters the structure and function of a protein by attaching a phosphate group to it (Siriwardena S U, Munkanatta Godage D N P, Shoba V M, et al. Phosphorylation-Inducing Chimeric Small Molecules. J Am Chem Soc. 2020; 142 (33): 14052-14057). Traditionally, small molecules have been used to inhibit enzyme function, but PHICS represents a novel approach that endows new functions to enzymes via proximity-mediated effects (Siriwardena, et al. 2020).

### 4. Methods of Detection

In example embodiments, detection of biomarkers in a tumor sample can be used to determine if subjects are resistant to a PARP inhibitor or have a worse prognosis due to increased VEGFR1 signaling (e.g., VEGFR/Flt1, PIGF, and/or activated AKT). Biomarkers in the context of the present invention encompasses, without limitation nucleic acids, proteins, reaction products, and metabolites, together with their polymorphisms, mutations, variants, modifications, subunits, fragments, and other analytes or sample-derived measures. The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such. The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

In example embodiments, the genes, biomarkers, and/or cells expressing biomarkers may be detected or isolated by immunofluorescence, immunohistochemistry (IHC), fluorescence activated cell sorting (FACS), mass spectrometry (MS), mass cytometry (CyTOF), sequencing, RNA-seq, single cell RNA-seq, quantitative RT-PCR, single cell qPCR, FISH, RNA-FISH, MERFISH (multiplex (in situ) RNA FISH), Nanostring (see e.g., Geiss G K, et al., Direct multiplexed measurement of gene expression with color-coded probe pairs. Nat Biotechnol. 2008 March; 26 (3): 317-25), in situ hybridization (ISH), CRISPR-effector system mediated screening assay (e.g., SHERLOCK assay), and any combination thereof. Other methods including absorbance assays and colorimetric assays are known in the art and may be used herein. In certain embodiments, a cancer is diagnosed, prognosed, or monitored. For example, a tissue sample may be obtained and analyzed for specific cell markers (IHC) or specific transcripts (e.g., RNA-FISH). In one embodiment, tumor cells are stained for VEGFR1/Flt1 or PGF or phosphorylated AKT. In one embodiment, the cells are fixed. In another embodiment, the cells are formalin fixed and paraffin embedded. Not being bound by a theory, the increased expression of VEGFR1/Flt1 or PGF or phosphorylated AKT indicate outcome and treatments.

In example embodiments, detection of a biomarker in a subject is compared to a reference value. In example embodiments, the reference value is a value of the biomarker determined or obtained for other cancer samples. In example embodiments, the cancer samples used for the reference value are of the same cancer type as the subject. In example embodiments, the reference value is the average value for a set of tumor samples of the same type. In example embodiments, the reference value is obtained from a set of tumor samples obtained from subjects resistant to a PARP inhibitor (i.e., in this case, expression or phosphorylation is considered increased if a value similar to resistant subjects is detected). In example embodiments, the reference value is obtained from a set of tumor samples obtained from subjects sensitive to a PARP inhibitor. In example embodiments, the reference value is one or more values obtained from the subject before treatment or at the time of the first treatment (e.g., PARP inhibitor treatment). In example embodiments, more than one reference value is used. For example, reference values are obtained during the course of treatment and the current value is compared to previous values. In example embodiments, reference values are obtained from a publicly available database of cancer samples (e.g., The Cancer Genome Atlas (TCGA)).

The present invention also may comprise a kit with a detection reagent that binds to one or more biomarkers or can be used to detect one or more biomarkers.

### Immunoassays

In example embodiments, immunoassays can be used to determine if subjects are resistant to a PARP inhibitor or have a worse prognosis due to increased VEGFR1 signaling. Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

### Histology

In example embodiments, histology of a tumor sample can be used to determine if subjects are resistant to a PARP inhibitor or have a worse prognosis due to increased VEGFR1 signaling. Histology, also known as microscopic anatomy or microanatomy, is the branch of biology which studies the microscopic anatomy of biological tissues. Histology is the microscopic counterpart to gross anatomy, which looks at larger structures visible without a microscope. Although one may divide microscopic anatomy into organology, the study of organs, histology, the study of tissues, and cytology, the study of cells, modern usage places these topics under the field of histology. In medicine, histopathology is the branch of histology that includes the microscopic identification and study of diseased tissue. Biological tissue has little inherent contrast in either the light or electron microscope. Staining is employed to give both contrast to the tissue as well as highlighting particular features of interest. When the stain is used to target a specific chemical component of the tissue (and not the general structure), the term histochemistry is used. Antibodies can be used to specifically visualize proteins, carbohydrates, and lipids. This process is called immunohistochemistry (IHC), or when the stain is a fluorescent molecule, immunofluorescence (IF). This technique has greatly increased the ability to identify categories of cells under a microscope. Other advanced techniques, such as nonradioactive in situ hybridization (ISH), can be combined with immunochemistry to identify specific DNA or RNA molecules with fluorescent probes or tags that can be used for immunofluorescence and enzyme-linked fluorescence amplification.

### Spatial Detection

In example embodiments, spatial detection of a tumor sample can be used to determine if subjects are resistant to a PARP inhibitor or have a worse prognosis due to increased VEGFR1 signaling. Methods of generating spatial data of varying resolution are known in the art, for example, ISS (Ke, R. el al. In situ sequencing for RNA analysis in preserved tissue and cells. Nat. Methods 10, 857-860 (2013)), MERFISH (Chen, K. H., Boettiger, A. N., Moffitt, J. R., Wang, S. & Zhuang, X. Spatially resolved, highly multiplexed RNA profiling in single cells. Science 348, (2015)), smFISH (Codeluppi, S. et al. Spatial organization of somatosensory the cortex revealed by cyclic smFISH. biorxiv.org/lookup/doi/10.1101/276097 (2018) doi: 10.1101/276097), osmFISH (Codeluppi, S. et al. Spatial organization of the somatosensory cortex revealed by osmFISH. Nat. Methods 15, 932-935 (2018)), STARMap (Wang, X. et al. Three-dimensional intact-tissue sequencing of single-cell transcriptional states. Science 361, eaat5691 (2018)), Targeted ExSeq (Alon, S. et al. Expansion Sequencing: Spatially Precise In Situ Transcriptomics in Intact Biological Systems. biorxiv.org/lookup/doi/10.1101/2020.05.13.094268 doi (2020) 10.1101/2020.05.13.094268), seqFISH+ (Eng, C.-H. L. et al. Transcriptome-scale superresolved imaging in tissues by RNA seqFISH+. Nature (2019) doi: 10.1038/s41586-019-1049-y.), Spatial Transcriptomics methods (e.g. Spatial Transcriptomics (ST)) (see, e.g., Stahl, P. L. et al. Visualization and analysis of gene expression in tissue sections by spatial transcriptomics. Science 353, 78-82 (2016)) (now available commercially as Visium); Visium Spatial Capture Technology, I OX Genomics, Pleasanton, CA; WO2020047007A2; WO2020123317A2; WO2020047005A1; WO2020176788A1; and WO2020190509A9), Slide-seq (Rodriques, S. G. et al. Slide-seq: A scalable technology for measuring genome-wide expression at high spatial resolution. Science 363, 1463-1467 (2019)), High Definition Spatial Transcriptomics (Vickovic, S. et al. High-definition spatial transcriptomics for in situ tissue profiling. Nat. Methods 16, 987-990 (2019)), or slide-tags (Russell A J C, Weir J A, Nadaf N M, et al. Slide-tags enables single-nucleus barcoding for multimodal spatial genomics [published correction appears in Nature. 2024 January; 625 (7994): E11). In certain embodiments, the spatial data can be immunohistochemistry data or immunofluorescence data.

### MS Methods

Biomarker detection may also be evaluated using mass spectrometry (MS) methods. In example embodiments, MS is used to detect biomarkers in non-invasive samples (e.g., blood or stool). A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS) n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)2 fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g., diabodies etc.) imprinted polymers, avimers, peptidomimetics, peptoids, peptide nucleic acids, threose nucleic acid, a hormone receptor, a cytokine receptor, and synthetic receptors, and modifications and fragments of these. Single cell sequencing

### Sequencing

In example embodiments, sequencing is used to identify expression of genes or transcriptomes in single cells (e.g., RNA-seq). In certain embodiments, sequencing comprises high-throughput (formerly “next-generation”) technologies to generate sequencing reads. Methods for constructing sequencing libraries are known in the art (see, e.g., Head et al., Library construction for next-generation sequencing: Overviews and challenges. Biotechniques. 2014; 56 (2): 61-77). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., cDNA) may include sequencing adaptors that are compatible with use in, e.g., Illumina's reversible terminator method, long read nanopore sequencing, Roche's pyrosequencing method (454), Life Technologies' sequencing by ligation (the SOLID platform) or Life Technologies' Ion Torrent platform. Examples of such methods are described in the following references: Margulies et al (Nature 2005 437:376-80); Schneider and Dekker (Nat Biotechnol. 2012 Apr. 10; 30 (4): 326-8); Ronaghi et al (Analytical Biochemistry 1996 242:84-9); Shendure et al (Science 2005 309:1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi: 10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12 (1): 44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx. doi. org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14 (3): 302-308, 2017; Cao, et al., Comprehensive single-cell transcriptional profiling of a multicellular organism. Science, 357 (6352): 661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14 (10): 955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; and Drokhlyansky E, Smillie C S, Van Wittenberghe N, et al. The Human and Mouse Enteric Nervous System at Single-Cell Resolution. Cell. 2020; 182(6): 1606-1622. e23, which are herein incorporated by reference in their entirety.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (IxSSC plus 0.2% SDS) followed by 10 minutes at 25° C. in high stringency wash buffer (0.1 SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992). Further embodiments are illustrated in the following Examples which are given for illustrative purposes only and are not intended to limit the scope of the invention.

### 5. Examples

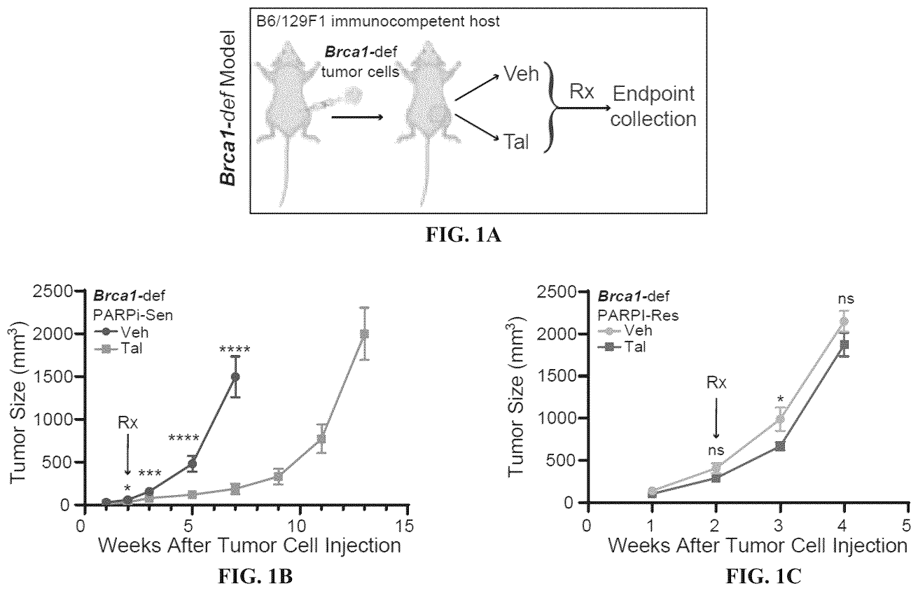

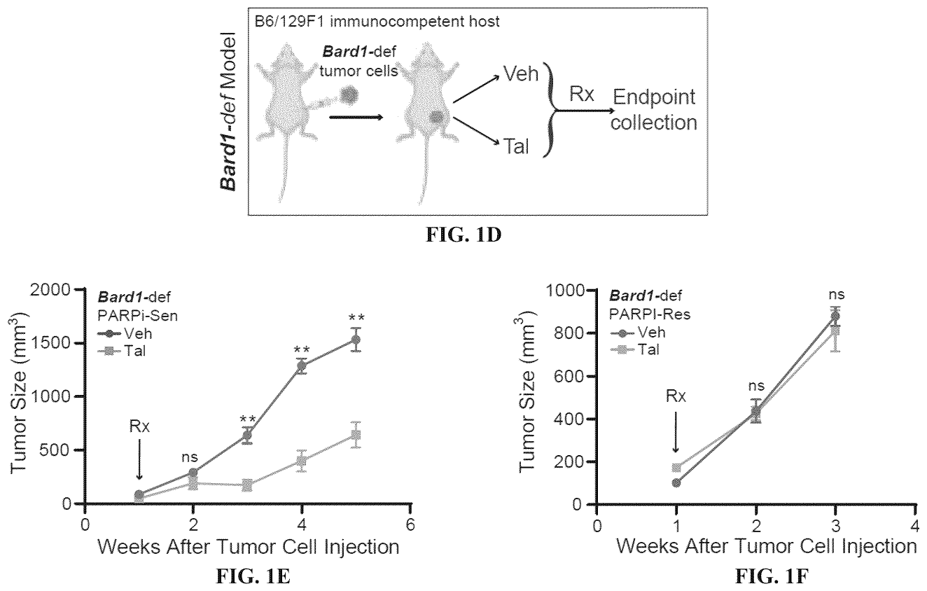

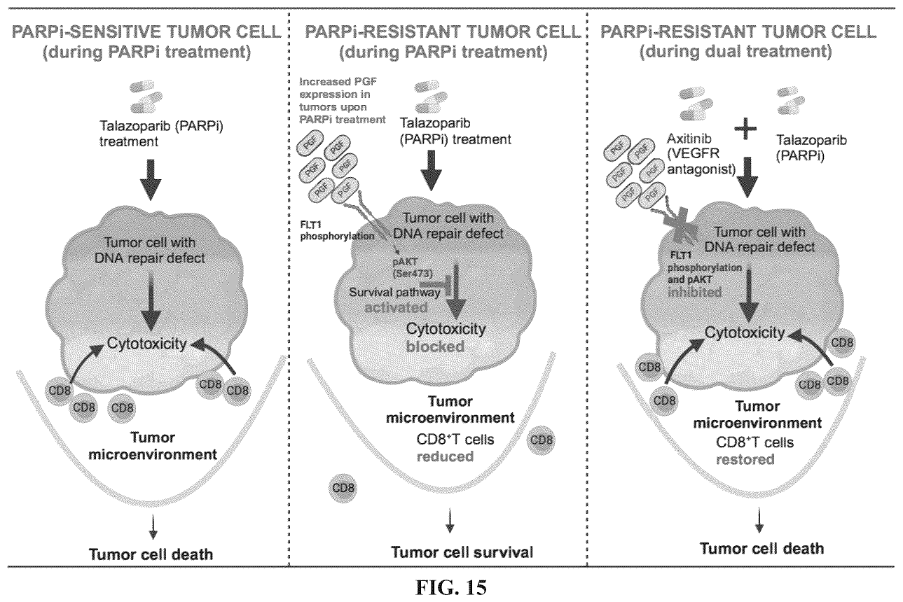

There are a variety of PARP resistance cancer treatment implementations. Notwithstanding, with reference to FIGS. 1 to 20 and for the exemplary purposes of this disclosure, many of the implementations relate to the following examples.

### Example 1

### Generation of In-Vivo PARPi-Response-and-Recurrence Models.

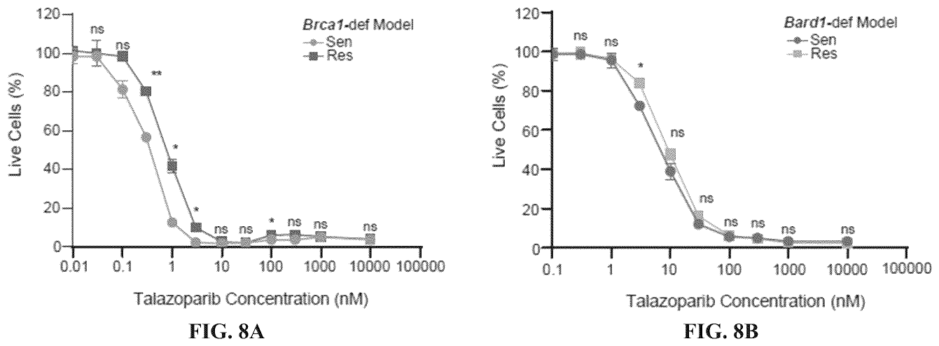

To study mechanisms of PARPi resistance in vivo, mammary glands from syngeneic B6/129F1 mice were orthotopically injected with luciferase-labeled Brca1- or Bard1-deficient mammary cancer cell lines (hereafter referred to as Brca1-def and Bard1-def) derived from Brca1- or Bard1-conditionally deleted mice²³ (FIGS. 1A and 1D). As tumors started to grow, tumor-bearing mice were randomized and treated five days/week with either vehicle or the PARPi talazoparib (referred to as “Tal” in the figures) at a dose of 0.3 mg/kg body weight/day starting at either 14 days (Brca1-def) or 7 days (Bard1-def) post tumor-cell injection. Tumor size was monitored weekly (FIGS. 1B and 1E). Treatment with talazoparib inhibited tumor growth in mice for several weeks in both Brca1-def and Bard1-def models; however, all talazoparib-treated tumors eventually developed drug resistance as demonstrated by the recurrence of tumors (FIGS. 1B and 1E). While tumor growth in Brca1-def models was monitored for 13 weeks post tumor-cell injection, Bard1-def models could only be monitored for 5 weeks post tumor-cell injection due to the onset of cachexia, as described in the previous studies³⁶. PARPi-resistant tumors were then collected, and cancer cells were purified and re-injected into the mammary glands of new B6/129F1 recipient mice. Mice were treated following the same dosing schedule as described above. Notably, in contrast to the PARPi-sensitive tumor lines (FIGS. 1B and 1E), PARPi-resistant tumor lines showed no tumor regression with PARPi treatment, confirming the acquisition of drug resistance (FIGS. 1C and 1F). Interestingly, while the difference in response to talazoparib between PARPi-sensitive and -resistant lines was striking in vivo, only a modest difference was observed in vitro (FIGS. 8A-B), suggesting that physiological context might be necessary for the manifestation of certain resistance mechanisms in response to PARP inhibition. These studies show that the Brca1-def and Bard1-def orthotopic allograft models recapitulate the distinct phases of the PARPi-therapy response observed in patients, which is characterized by a striking initial response to PARPi treatment followed by the gradual development of resistance and eventual recurrence of tumors.

### PARPi-Resistant Tumors Show Increased VEGFR2 and PIGF Expression but Only Modest Sensitization to PARPi Upon VEGFR2 Depletion.

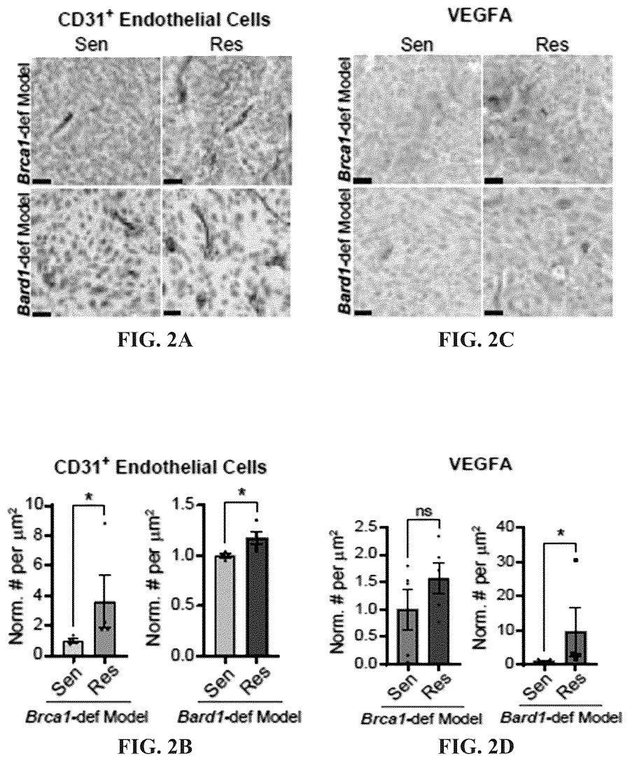

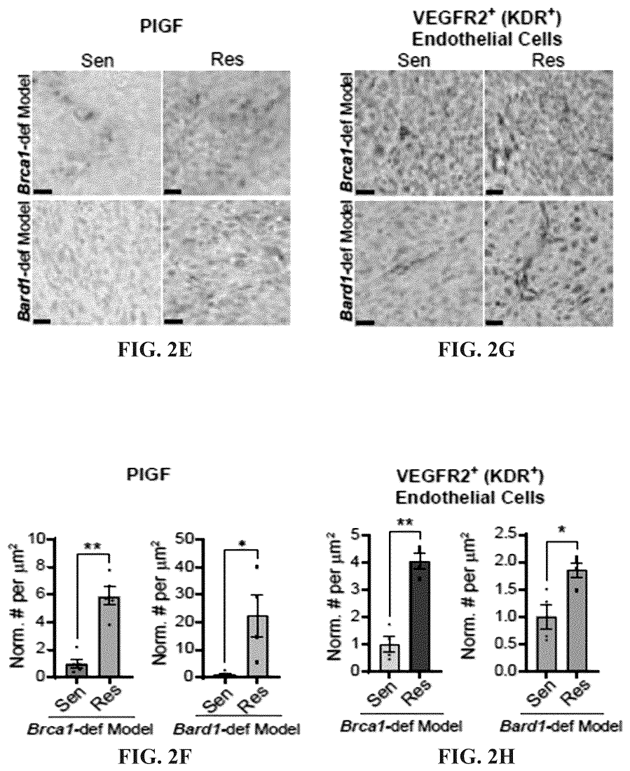

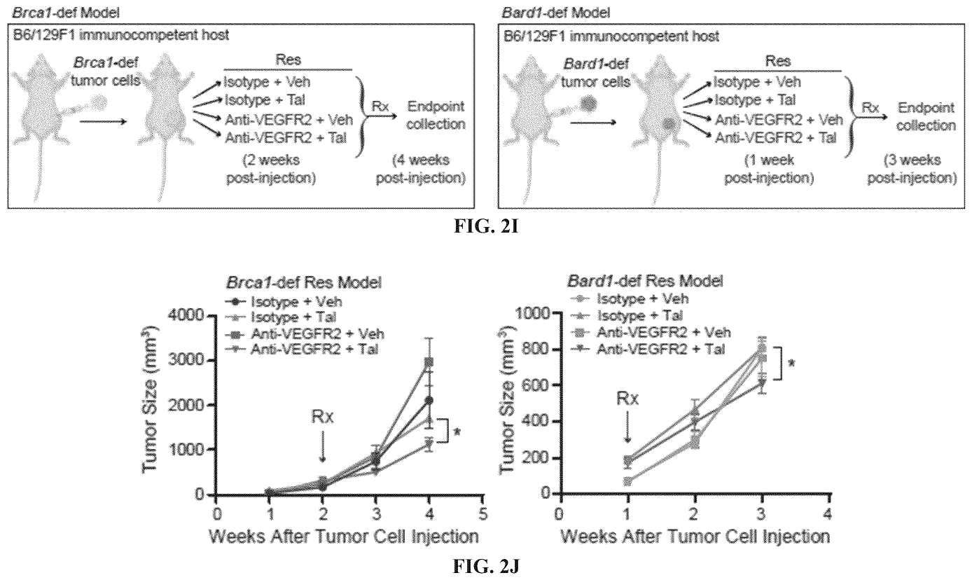

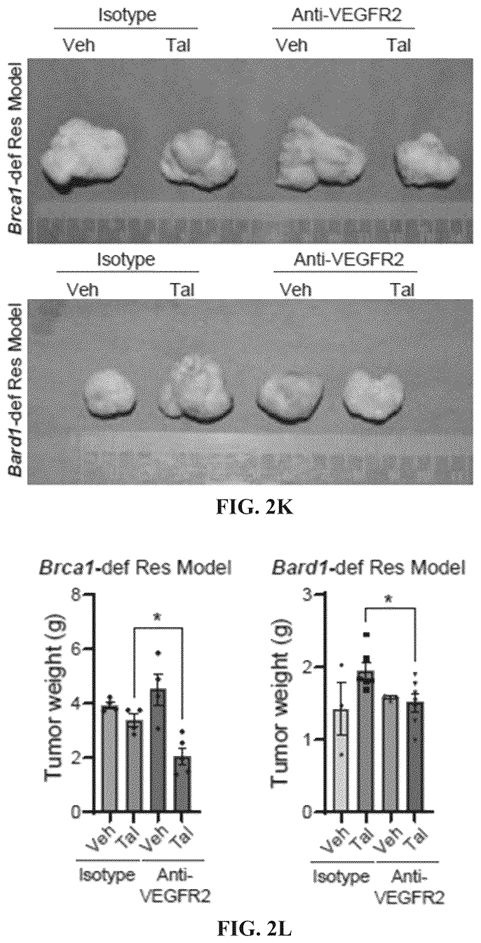

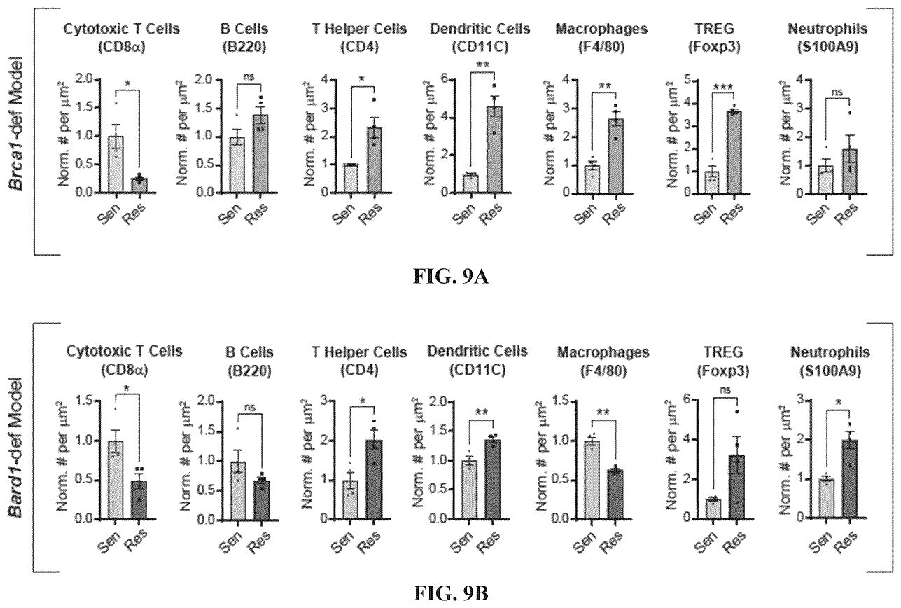

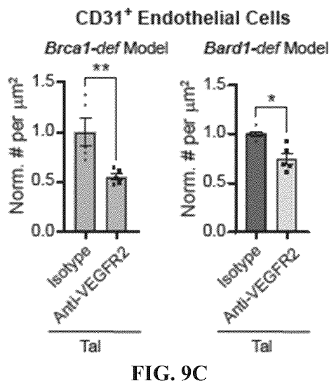

Since PARPi resistance was more faithfully recapitulated in the Brca1-def and Bard1-def models in vivo than in vitro (FIG. 1 and FIG. 8), Applicants hypothesized that signals from the tumor microenvironment are important for PARPi resistance. To identify mechanisms of PARPi resistance in the Brca1-def and Bard1-def models, Applicants analyzed differences in the tumor microenvironment components from both PARPi-sensitive and -resistant tumors by immunohistochemical staining (FIGS. 2A-H and FIGS. 9A-B). Applicants observed that PARPi-resistant tumors show a significant increase in CD31⁺ blood vessels (FIGS. 2A-B), indicating a greater amount of angiogenesis in PARPi-resistant tumors compared to PARPi-sensitive tumors. An analysis of the major immune cell populations revealed a significant reduction in the number of CD8⁺ T cells and an increase in both CD4⁺ helper-T cells and CD11C⁺ dendritic cells in PARPi-resistant compared to PARPi-sensitive tumors (FIGS. 9A-B). These results suggest that changes in angiogenesis and immune-cell composition may underlie PARPi resistance in BRCA1-mutant breast tumors.

Since the vascular endothelial growth factor (VEGF) signaling pathway regulates both angiogenesis and the immune response^{37,38}, Applicants performed immunohistochemical staining to determine whether the VEGF pathway is activated in the PARPi-resistant tumors. Among the VEGF family of ligands, VEGFA expression showed a modest increase while placental growth factor (PIGF) showed a striking increase (Brca1-def: 5.9 fold, p=0.0079 and Bard1-def: 22.5 fold, p=0.0159 in PIGF) in the PARPi-resistant tumors (FIGS. 2C-F). There were no differences in VEGFB, C and D expression between PARPi-sensitive and -resistant tumors (data not shown). These results indicate that signaling through the VEGF family of ligands could be important for PARPi resistance.

VEGFR2 (also known as KDR) is a key receptor in vascular endothelial cells that binds to the VEGF family of ligands and serves as a major signal transducer for angiogenesis³⁹. Consistent with the increase in blood vessels (FIGS. 2A-B), the number of VEGFR2-expressing endothelial cells also significantly increased in the PARPi-resistant tumors compared to PARPi-sensitive tumors (FIGS. 2G-H). VEGFR2 expression was primarily detected in the endothelial cells (as expected) with no expression in tumor cells by immunohistochemical analysis (FIG. 2G). Since PARPis can synergize with anti-angiogenic agents in ovarian cancer⁴⁰, Applicants examined whether blocking pro-angiogenic VEGFR2 signaling could sensitize PARPi-resistant breast tumors to talazoparib. To this end, Applicants implanted PARPi-resistant tumor cells from the Brca1-def and Bard1-def models in the mammary glands of new recipient mice and treated them with either VEGFR2 antibody (200 μg/mouse three times a week) or isotype control antibody in combination with vehicle or talazoparib (schematic in FIG. 2I). VEGFR2 blockade in combination with talazoparib reduced the number of CD31⁺ vessels in PARPi-treated tumors in both the Brca1-def and Bard1-def models (FIG. 9C), which confirmed the efficacy of the VEGFR2 antibody treatment. Interestingly, despite reduced angiogenesis, VEGFR2 inhibition in combination with talazoparib only modestly reduced tumor growth in the Brca1-def and Bard1-def models (FIGS. 2J-L), suggesting that other mechanisms are important for driving PARPi resistance in these breast cancer models.

### FLT1/VEGFR1 in Breast Cancer Cells Promotes PARPi Resistance.

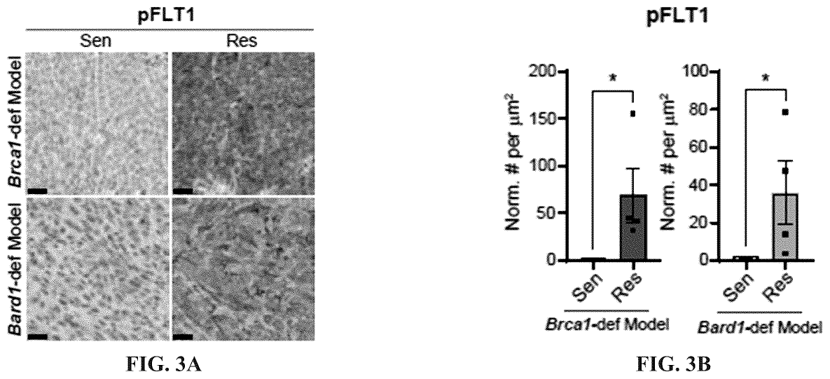

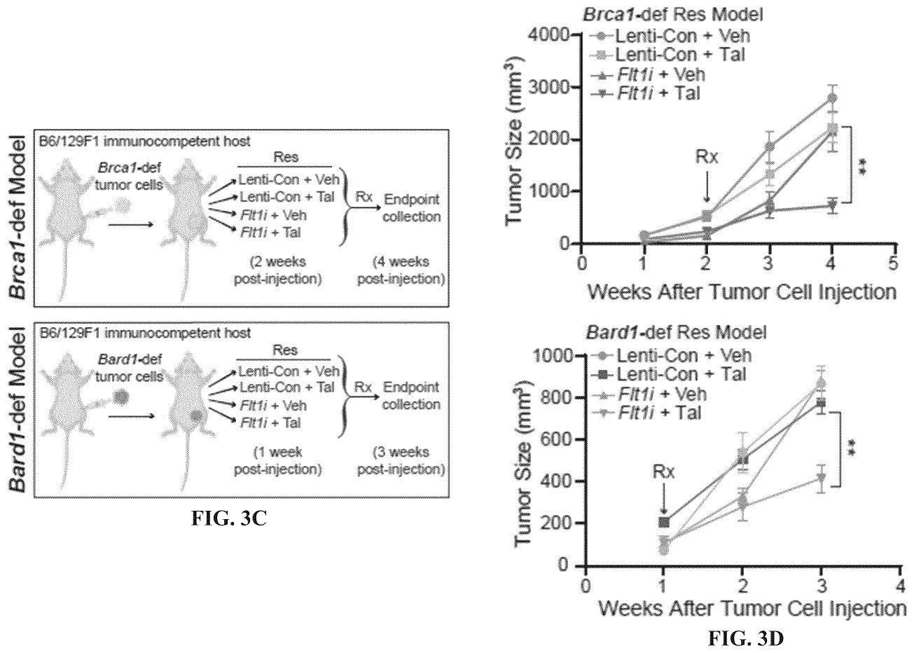

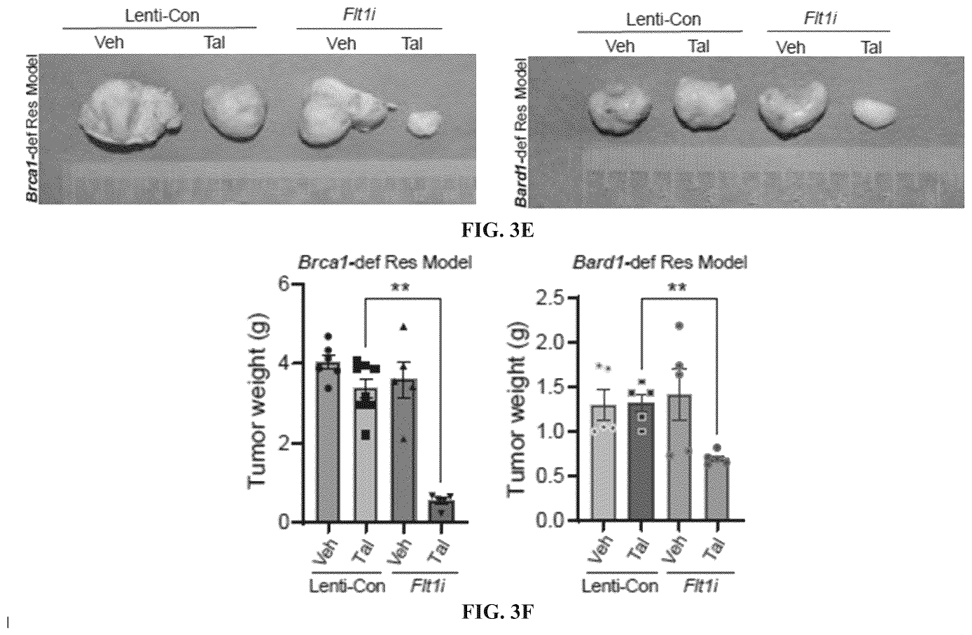

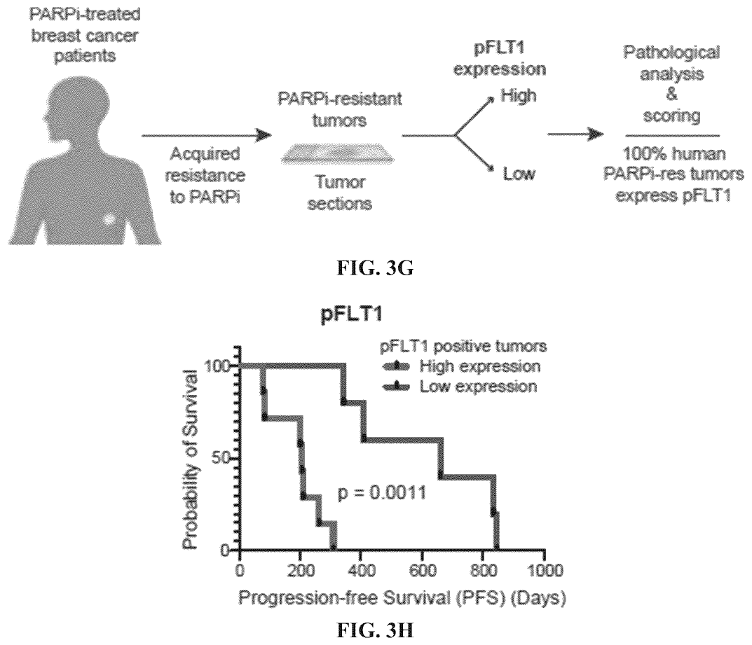

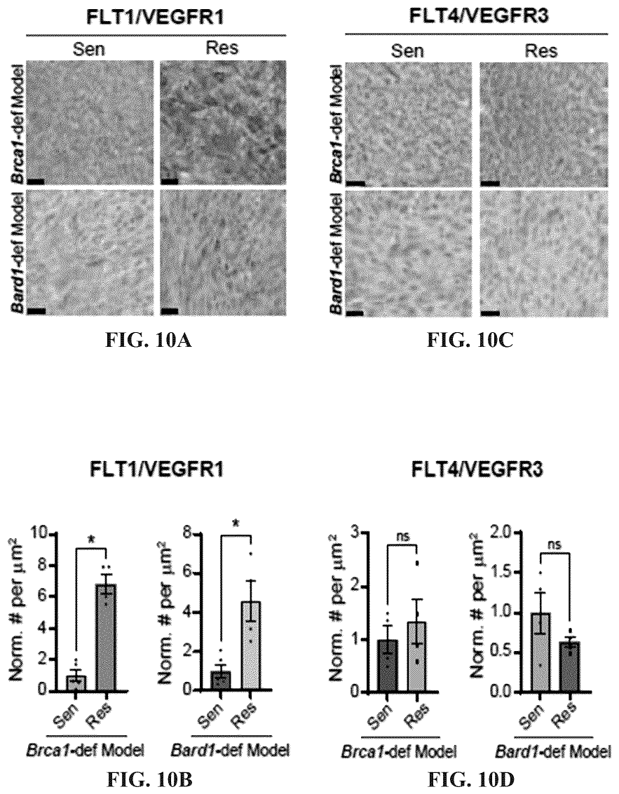

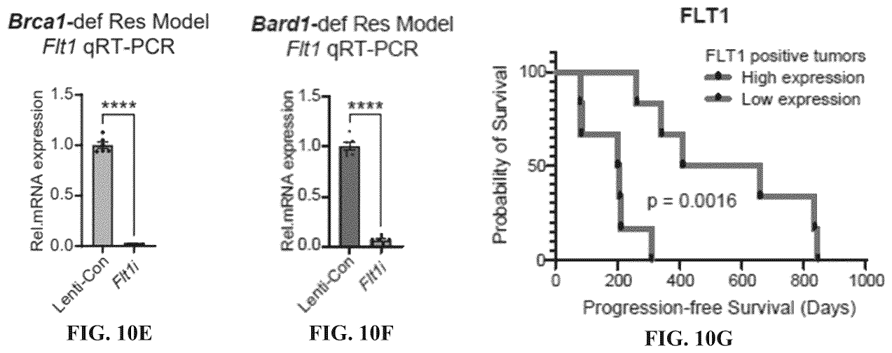

The high expression of PIGF in the PARPi-resistant tumors in the Brca1-def and Bard1-def models (FIGS. 2E-F) prompted Applicants to investigate whether the PIGF receptor FLT1 (also known as VEGFR1) contributes to PARPi resistance. Indeed, Applicants observed a striking increase in levels of both activated FLT1 (phospho-Tyr1213, referred to as pFLT1 hereafter) and total FLT1 in the cancer cells from PARPi-resistant tumors compared to PARPi-sensitive tumors by immunohistochemical analysis (FIGS. 3A-B and FIGS. 10A-B). No significant changes in FLT4 (also known as VEGFR3) expression were observed in the PARPi-resistant compared to PARPi-sensitive tumors (FIGS. 10C-D). These observations raised the possibility that increased PIGF in the tumor microenvironment upon PARPi treatment activates FLT1 signaling in cancer cells, which could contribute to PARPi resistance. To test this hypothesis, Applicants performed loss-of-function experiments to determine whether genetic repression of Flt1 in PARPi-resistant tumor cells from the Brca1-def and Bard1-def models impacts tumor growth either alone or in the presence of PARPis. Applicants first engineered lentiviruses to repress Flt1 in the cancer cells using CRISPR-mediated gene repression (CRISPRi) (FIGS. 10E-F) following the previous studies⁴¹. Next, Applicants injected PARPi-resistant Brca1-def and Bard1-def tumor cells, which were transduced with either control lentivirus (“Lenti-Con”) or lentivirus encoding guide RNA for Flt1 (“Flt1i”), via orthotopic injection into the mammary gland of syngeneic B6/129F1 mice. As described in the schematic in FIG. 3C, mice were randomized and treated five days/week with either vehicle or talazoparib (0.3 mg/kg body weight/day). An analysis of tumor growth and weight at endpoint revealed that Flt1 repression in cancer cells re-sensitizes PARPi-resistant tumors to talazoparib without impacting tumor growth in the absence of talazoparib treatment (FIGS. 3D-F).

Applicants next examined whether human breast cancers that progress during PARPi treatment express FLT1 in the cancer cells from PARPi-resistant tumors (FIGS. 3G-H and Table 1). To this end, Applicants analyzed FLT1 expression by immunohistochemistry of tumor tissue sections collected following the development of PARPi resistance in breast cancer patients harboring mutations in the BRCA1, BRCA2, or PALB2 (Partner and Localizer of BRCA2) DNA damage response (DDR) genes. Consistent with the murine models, 100% of these PARPi-resistant tumors (n=12) expressed FLT1 (both phospho-Tyr1213 and total FLT1). Further stratification of these tumors by FLT1 expression levels revealed that the tumors expressing high levels of pFLT1 progressed faster than tumors with low expression of pFLT1 (p=0.0011 for pFLT1, FIG. 3H, and 0.0016 for total FLT1, FIG. 10G; see also Table 1). These findings underscore the importance of a previously overlooked role for cancer-cell-derived FLT1 in promoting PARPi resistance and cancer progression in breast cancer patients.

TABLE 1
De-identified list of tumor tissue samples from PARPi-treated human
breast cancer patients (n = 12) with confirmed PALB2, or BRCA1/2
germline mutation collected at the time of acquired resistance.

| Sample ID | Germline Variant |
|---|---|
| S21-5008 | PALB2 |
| S20-36503 | BRCA1 |
| S21-43296 | BRCA2 |
| S21-28249 | BRCA2 |
| S20-47493 | BRCA1 |
| S18-64718 | BRCA2 |
| S18-55678 | BRCA2 |
| S20-33375 | BRCA2 |
| S21-42534 | BRCA1 |
| S20-4959 | BRCA2 |
| PARP-008 | BRCA1 |
| S19-51283 | BRCA1 |

### The Pan-VEGFR Blocker Axitinib Re-Sensitizes PARPi-Resistant Breast Tumors to PARPi Treatment.

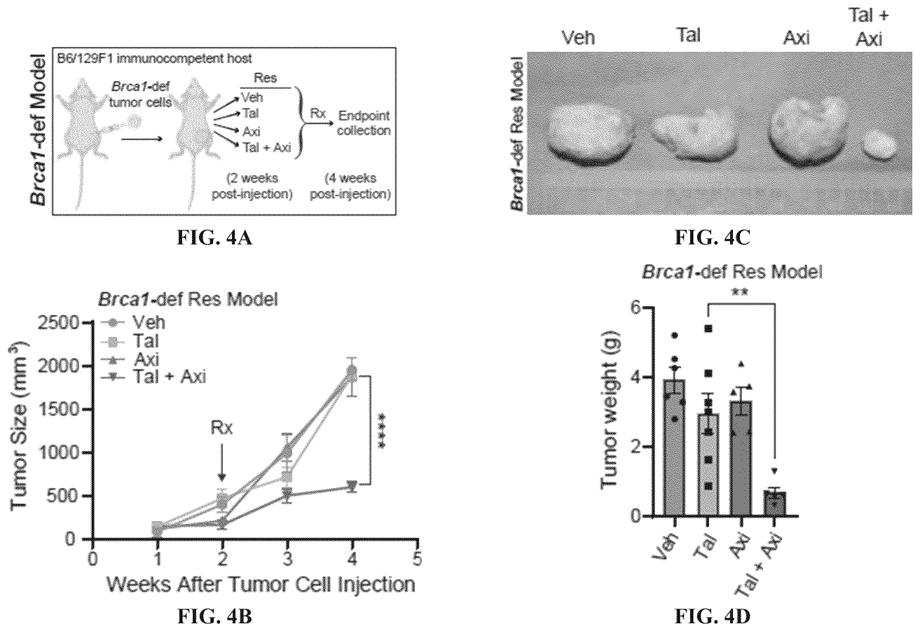

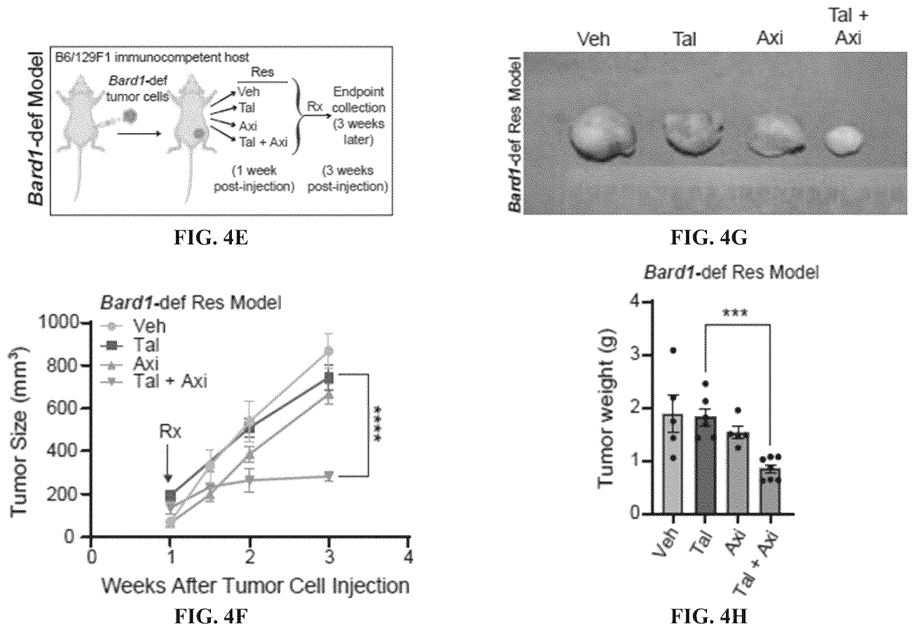

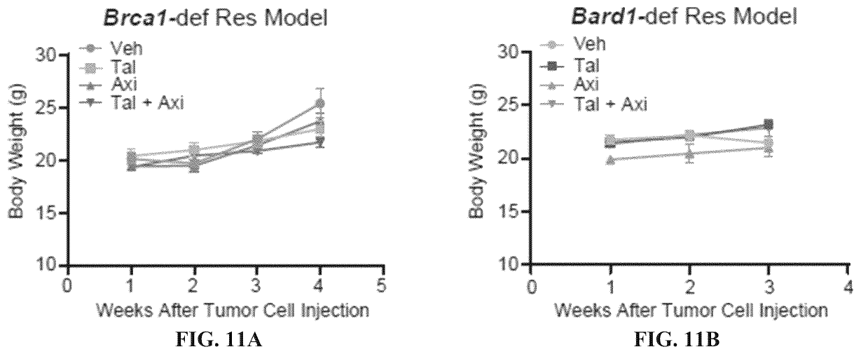

Applicants next tested whether pharmacologically blocking FLT1 signaling with the pan-VEGFR1/2/3 inhibitor axitinib could resensitize the PARPi-resistant tumors to talazoparib treatment. Axitinib is an FDA-approved drug for treating metastatic renal-cell carcinoma patients⁴². To this end, Applicants orthotopically injected PARPi-resistant Brca1-def and Bard1-def cells into the mammary gland of syngeneic B6/129F1 mice (see schematic in FIGS. 4A and 4E). Mice were randomized and treated five days/week with either vehicle, talazoparib (0.3 mg/kg body weight/day), axitinib (30 mg/kg body weight/day), or talazoparib plus axitinib (0.3 mg/kg body weight/day and 30 mg/kg body weight/day, respectively). Compared to the single-treatment groups, talazoparib-plus-axitinib treatment led to a striking reduction in PARPi-resistant tumor burden (FIGS. 4B-D and 4F-H). None of the treatments led to overt toxicities, and stable body weight was maintained by all mice for the duration of the studies (FIGS. 11A-B). Based on the genetic and pharmacological inhibition studies, blocking both FLT1 and PARP could be beneficial for controlling breast tumor growth in BRCA1-mutant breast cancer. FLT1 activation in cancer cells induces pro-survival AKT signaling that counteracts PARPi-induced cell death.

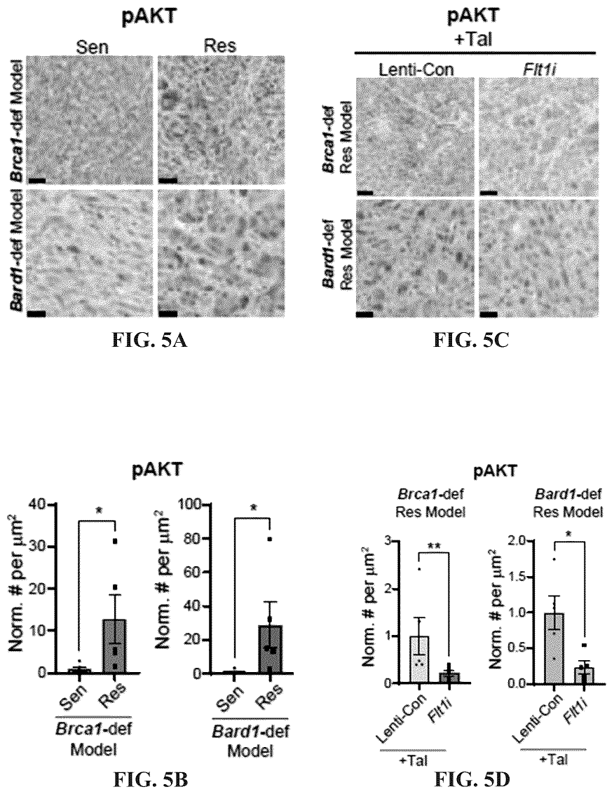

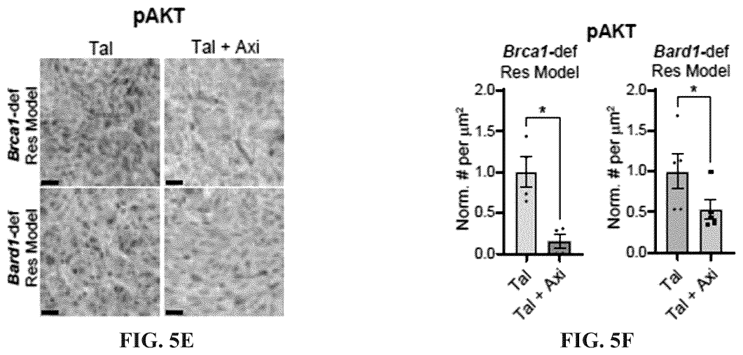

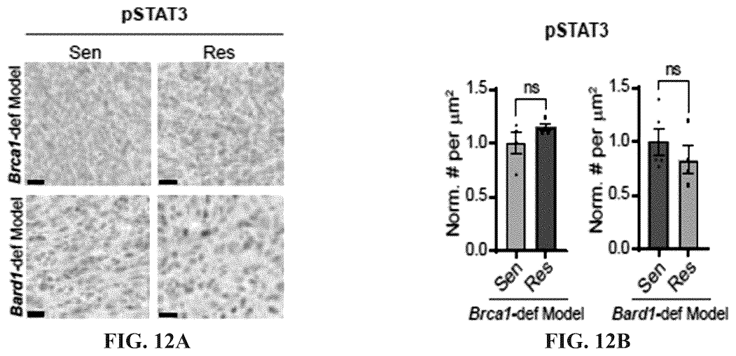

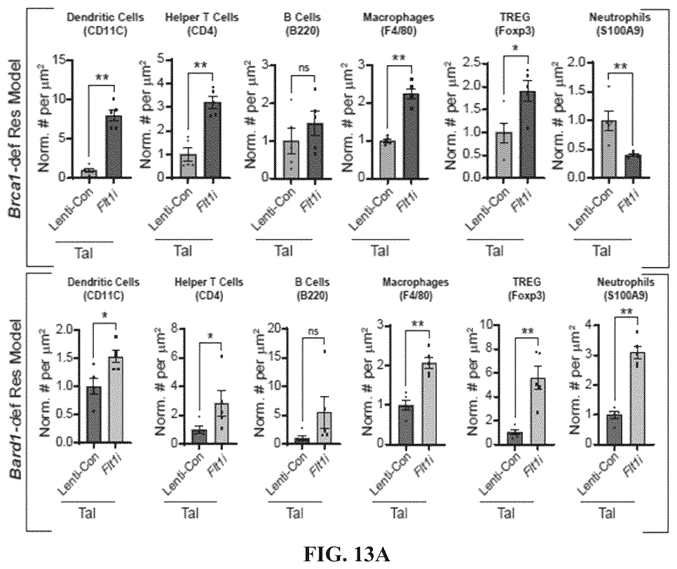

To investigate how FLT1 activation in breast cancer cells counteracts PARPi-induced cytotoxicity, Applicants examined the FLT1-downstream pathways activated in PARPi-resistant tumors that could inhibit PARPi-induced cell death. Upon binding to VEGF-family ligands (e.g., PIGF), FLT1 activates growth and survival pathways, including AKT and STAT3 signaling, in immune and vascular smooth muscle cells⁴³⁻⁴⁷. Indeed, Applicants found that AKT, but not STAT3, is significantly activated in PARPi-resistant tumors compared to PARPi-sensitive tumors in the Brca1-def and Bard1-def models (FIGS. 5A-B and FIGS. 12A-B). Importantly, the experimental combinations of both talazoparib-plus-Flt1i and talazoparib-plus-axitinib in PARPi-resistant tumors significantly dampened AKT activation (FIGS. 5C-F). These results suggest that FLT1-AKT pathway activation in BRCA1-mutant breast cancer cells serves as a mechanism to escape PARPi-induced cytotoxicity in vivo.

### Cytotoxic Immune Response Restored by the Combination of PARPi and FLT1 Blockade.

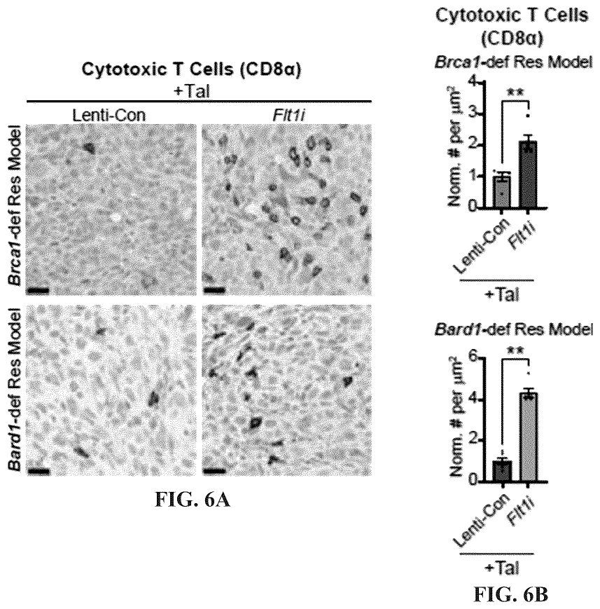

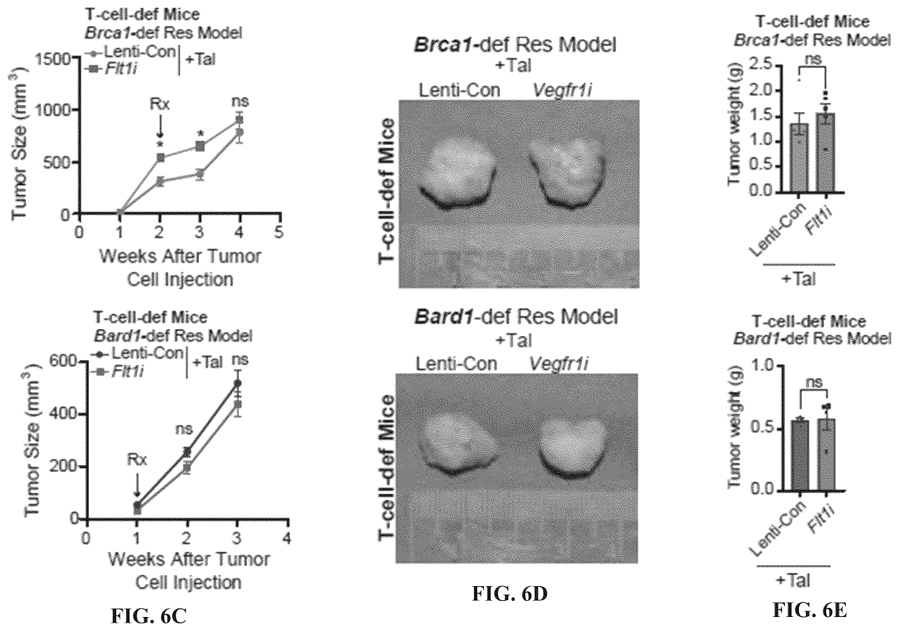

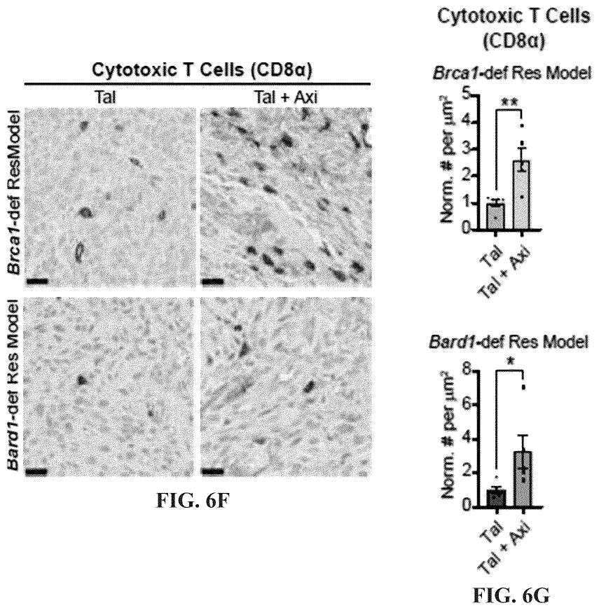

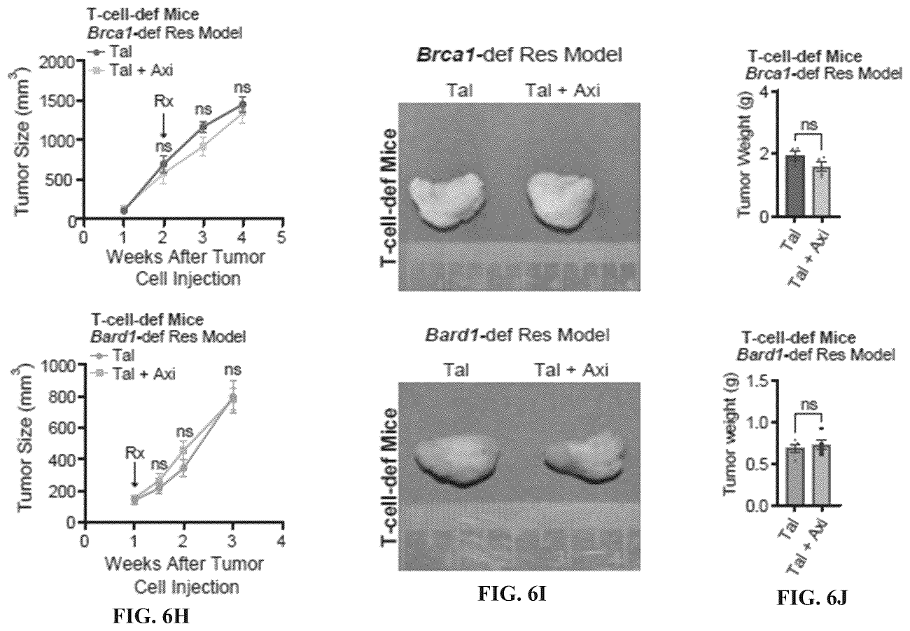

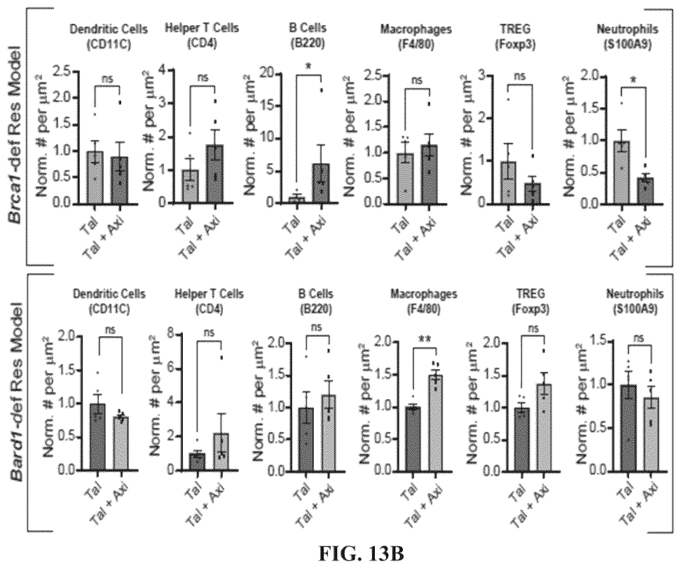

Based on the observation that PARPi-resistant tumors exhibit a reduced number of CD8⁺ T cells (FIGS. 9A and B) and on knowledge of the immune-modulatory functions of the VEGF signaling pathway³⁷, Applicants asked whether inhibiting FLT1 signaling could indirectly impact the number of CD8⁺ T cells, and other immune cells, in PARPi-resistant tumors (FIG. 6). Consistently, both genetic and pharmacological blockade of FLT1 signaling in combination with talazoparib treatment increased CD8⁺ T-cell infiltration into PARPi-resistant tumors in both the Brca1-def and Bard1-def models as determined by immunohistochemical staining (FIGS. 6A-B and FIGS. 6F-G). Using T-cell-deficient mice, Applicants next investigated whether the tumor regression observed in PARPi-resistant tumors upon FLT1 blockade is T-cell-dependent. Indeed, the tumor regression observed in B6/129F1 immunocompetent hosts with genetic and pharmacological FLT1 blockade combined with talazoparib treatment (FIGS. 3D-F and FIG. 4) was no longer observed in the T-cell-deficient mice (FIGS. 6C-E and FIGS. 6H-J). Among other immune cells, CD4⁺ helper T cells, B220⁺ B cells and F4/80⁺ macrophages showed a trend towards increased tumor infiltration upon FLT1 blockade in the Brca1-def and Bard1-def models (FIGS. 13A-B). These results demonstrate that the sensitization of PARPi-resistant tumors with FLT1 blockade is T-cell-dependent, and multiple immune changes in the tumor microenvironment might be associated with PARPi resistance, which remains to be investigated in future studies.

Association of FLT1 Activation in Cancer Cells at Pre-Treatment with Faster Progression on PARP Inhibitors in Breast Cancer Patients.

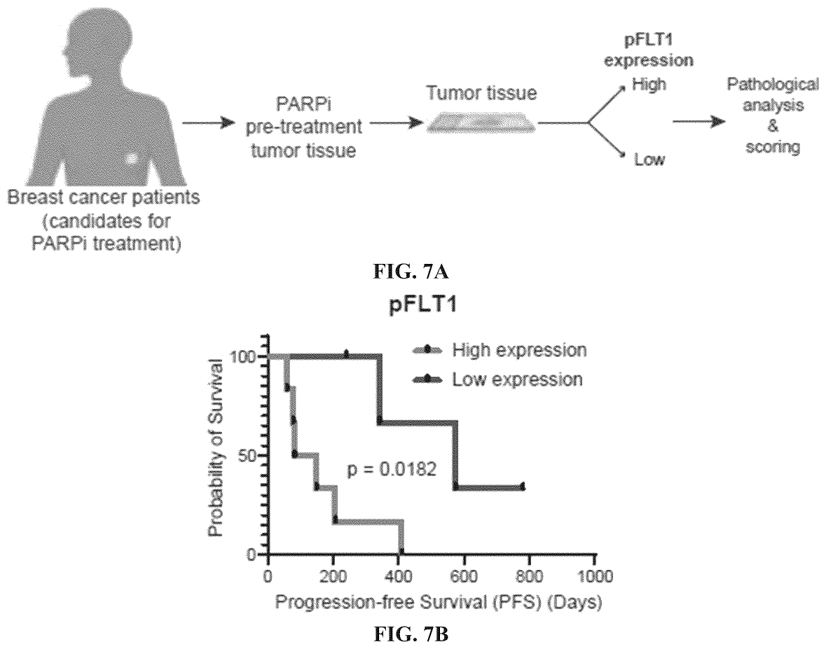

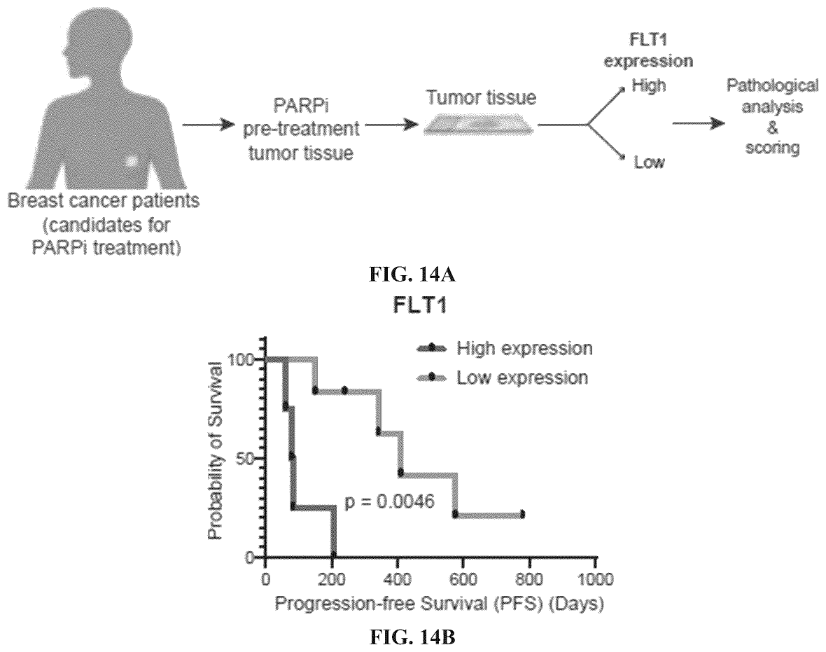

To clinically validate the preclinical findings, Applicants performed FLT1 and phospho-FLT1 immunostaining on tissue specimens that were obtained prior to PARPi treatment from 10 breast cancer patients harboring mutations in the BRCA1/BRCA2/PALB2 DNA damage response (DDR) genes (FIG. 7, FIG. 14, and Table 2). The immunostained samples were scored as either FLT1-high or FLT1-low expression in cancer cells. Consistent with the preclinical observations (FIGS. 3-4), an independent blinded pathological examination revealed a statistically significant association between FLT1 expression (both activated and total) at pre-treatment and shorter progression-free survival (p=0.0182 for pFLT1 and 0.0046 for total FLT1, FIGS. 7A-B and FIGS. 14A-B) in breast cancer patients. These findings suggest that expression of both pFLT1 and total FLT1 in breast cancer cells (pre-treatment) is significantly associated with higher risk of progression on PARPi, and thus, FLT1/pFLT1 expression status in human breast tumors with BRCA1, BRCA2 or PALB2 mutations could identify patients who might benefit from combination treatment with a PARPi and the VEGFR inhibitor axitinib.

TABLE 2
De-identified list of tumor tissue samples from human breast
cancer patients (n = 10) with confirmed PALB2, or BRCA1/2
germline mutation collected pre-PARP inhibitor treatment.

| Sample ID | Germline Variant |
|---|---|
| PARP 007 | BRCA1 |
| PARP 12 | BRCA1 |
| PARP 21 | BRCA1 |
| PARP 50 | BRCA2 |
| S19-30556 | PALB2 |
| S19-46410 | BRCA2 |
| S19-713 | BRCA2 |
| S18-73798 | BRCA2 |
| S19-15838 | BRCA1 |
| S17-58773 | BRCA2 |

### Methods

Animal Studies. The treatment of mice in this study were conducted in compliance with ethical regulations and guidelines set forth by the Columbia University Institutional Animal Care and Use Committee (IACUC), the U.S. National Research Council's Guide for the Care and Use of Laboratory Animals, and the U.S. Public Health Service's Policy on Humane Care and Use of Laboratory Animals. The Institutional guidelines of Columbia University Medical Center (CUMC) Institute of Comparative Medicine were followed in these studies. Mice were maintained in the CUMC barrier facility under conventional conditions with constant temperature and humidity and fed a standard diet (Labdiet 5053). Female B6129SF1/J mice purchased from the Jackson Laboratory and athymic mice aged 8 to 9 weeks purchased from Envigo were used in this study. These mice were injected with 5×10⁵ Brca1-def or Bard1-def cell lines and their derivatives into the mammary fat pad. To monitor tumor growth, bioluminescence imaging was performed weekly using the In Vivo Imaging System (IVIS, PerkinElmer). Briefly, mice were anesthetized with 3% to 4% isoflurane and injected with 1.5 mg of D-Luciferin (Fisher Scientific) via intraperitoneal injections. The mice were then placed inside the PerkinElmer IVIS Spectrum Optical Imaging System to measure bioluminescence and total photon flux was calculated with Living Image 4.7.3 software (PerkinElmer). Tumor growth was also monitored weekly by using an electric caliper to measure the length and width of the tumors in millimeters. The tumor volume can be calculated using the formula (length*(width²))/2, where length and width represent the longest and shortest dimensions of the tumor, respectively. Mice were weighed weekly, monitored twice a week, and were euthanized in accordance with the IACUC guidelines from Columbia University. The criteria for prompt euthanasia included weight loss of more than or equal to 20%, body-conditioning score (BCS) of 2 or less, signs of hunched posture, impaired locomotion, or respiratory distress. Mice with a tumor mass larger than 2 centimeters in diameter were also euthanized. Euthanasia was conducted by carbon dioxide inhalation with a secondary method of cervical dislocation. Timed collection of tumors was performed in matching cohorts and have been described in the respective figure legends.

To generate Brca1-def and Bard1-def resistant cell lines in vivo, Brca1-def and Bard1-def parental cells from primary tumors (which had not been exposed to talazoparib in culture), were injected into the mammary fat pad of B6129SF1/J mice at a concentration of 5×10⁵ cells per mouse. Long-term talazoparib (Selleckchem) treatment via oral gavage was initiated at 0.3 mg/kg/day, administered five days a week. Talazoparib was solubilized in N,N-dimethylacetamide (Millipore), and then diluted in 6% Kolliphor® HS 15 to make the working solution as vehicle. For the Brca1-def model, treatment started at two weeks post tumor cell injection. Talazoparib-resistant cancer cell lines were collected 13 weeks after tumor-cell injection. For the Bard1-def model, treatment started at one-week post tumor-cell injection and talazoparib-resistant cancer cell lines were collected 5 weeks after injection. To culture the harvested tumors, tumors were enzymatically dissociated using Dispase II (1 unit/mL, Roche) with 2 mg/mL collagenase Type I (Worthington). Both Brca1-def and Bard1-def parental cell lines used in this study were stably infected with lentivirus expressing luciferase enzyme and a hygromycin resistance marker, as previously described¹. Non-tumor cells were eliminated by supplementing the culturing medium with 200 μg/mL of hygromycin. A part of the tumor was fixed in 4% paraformaldehyde in PBS for 24 hours at 4° C., washed, and subsequently processed for histology.

For drug treatments, 5×10⁵ cells per mouse of resistant cells for both models were injected into the mammary fat pad of B6129SF1/J mice. For the Brca1-def model, talazoparib treatment (at 0.3 mg/kg/day, administered five days a week) started at 2 weeks following tumor cell injection, whereas for the Bard1-def model, treatment started at one-week following tumor cell injection. Mice were euthanized at their respective end points: week 4 for the Brca1-def model and week 3 for the Bard1-def model. For the in vivo VEGFR2 (KDR) inhibition, the VEGFR2 antibody (BE0060, BioXCell) treatment or the isotype control (anti-HRP) (BE0060, BioXCell) were diluted in a buffer (IP0070, BioXCell). Mice were randomly assigned to receive either 200 g of VEGFR2 antibody or the same amount of isotype control via intraperitoneal injection twice a week. Mice were euthanized at week 4 for the Brca1-def model and week 3 for the Bard1-def model, and their tumors were collected at their end points as shown in FIG. 2I. These tumors were fixed and processed as described above.

For the experiments checking the efficacy of talazoparib in resistant cells of both models expressing Lenti-Con or Flt1i/Vegfr1i, talazoparib treatment via oral gavage was initiated at 0.3 mg/kg/day, administered five days a week. For the Brca1-def model, treatment started at 2 weeks following tumor cell injection, and for the Bard1-def model, treatment was started one-week following tumor cell injection. Mice were euthanized at their respective end points: week 4 for the Brca1-def model and week 3 for the Bard1-def model. Tumors were collected and subsequently fixed and processed as described above.

To pharmacologically inhibit VEGFRs using axitinib, mice bearing resistant tumors from Brca1- and Bard1-def models were randomized into multiple treatment groups. Talazoparib was solubilized in N,N-dimethylacetamide (Millipore) and then diluted in 6% Kolliphor® HS 15. Axitinib (Selleckchem) was solubilized in 0.5% carboxymethylcellulose (w/v %)). Drugs were administered in mice by oral gavage five days a week with a dose of 0.3 mg/kg/day of talazoparib and 30 mg/kg/day of axitinib. Treatments started at two weeks following tumor cell injection for the Brca1-def model, and one week following tumor cell injection for the Bard1-def model. Brca1-def tumors were collected at 4 weeks post tumor cell injection and Bard1-def tumors were collected at 3 weeks post tumor cell injection. Tumors were collected and subsequently fixed and processed as described above.

Immunostaining analysis. Paraffin-embedded tumors from mice were sectioned at 5 μm thickness. Slides were baked at 60° C. for 1 hour and deparaffinized, rehydrated, and treated with 1% hydrogen peroxide for 10 minutes. Antigen retrieval was performed using either pH 6.0 citrate buffer (Vector Laboratories) or pH 9.0 Tris-based buffer (Vector Laboratories) in a steamer apparatus for 30 minutes, and endogenous avidin and biotin were blocked using avidin- and biotin-blocking reagents (Vector Laboratories), respectively. The slides were further blocked with BSA and goat or rabbit serum, and tissue sections were incubated with primary antibodies, including antibodies against phospho-AKT (S473) (1:100, #4060, Cell Signaling Technology), KDR/VEGFR2 (1:2000, #9698, Cell Signaling Technology), VEGFA (1:300, #AF-493-NA, R&D Systems), PIGF (1:300, AF465, R&D Systems), CD8a (1:200, #98941, Cell Signaling Technology), F4/80 (1:500, #70076, Cell Signaling Technology), B220 (1:400, #553085, BD Pharmingen), CD4 (1:200, #25229, Cell Signaling Technology), CD11C (1:250, #97585, Cell Signaling Technology), FOXP3 (1:100, #12653, Cell Signaling Technology), murine S100A9 (1:1000, #73425, Cell Signaling Technology), FLT4/VEGFR3 (1:250, #AF743, R&D Systems), phospho-Stat3 (S727) (1:100, #9134, Cell Signaling Technology) followed by incubation with the corresponding biotinylated secondary antibodies (1:250, Vector Laboratories). The ABC kit and DAB kit (Vector Laboratories) were used for detection following the manufacturer's instructions. Sections were subsequently counterstained with hematoxylin, dehydrated, and mounted using Cytoseal XYL (Richard-Allan Scientific) for subsequent histologic analysis.

Automated immunostaining for CD31, p-FLT1/p-VEGFR1, FLT1/VEGFR1 was performed at the Molecular Cytology Core Facility at MSKCC and processed as described below. Paraffin-embedded tissue sections were cut at 5 μm and heated at 58° C. for 1 hour. Samples were loaded into Leica Bond RX and sections were dewaxed at 72° C. before being pretreated with EDTA-based epitope retrieval ER2 solution (Leica, AR9640) for 20 minutes at 100° C. The rabbit polyclonal antibodies against CD31 (0.08 ug/ml, Abcam, ab182981), p-FLT1/p-VEGFR1 (1:50, Millipore, 07-758), or FLT1/VEGFR1 (2.5 ug/ml, Invitrogen, MA5-32045) were incubated for 60 minutes. Samples were then incubated with Leica Bond Post-Primary reagent (rabbit anti-mouse linker) (included in Polymer Refine Detection Kit (Leica, DS9800)) for 8 minutes, followed by incubation with Leica Bond Polymer (anti-rabbit HRP) (included in Polymer Refine Detection Kit (Leica, DS9800)) for another 8 minutes. Mixed DAB reagent (Polymer Refine Detection Kit) was then incubated for 10 minutes, and hematoxylin (Refine Detection Kit) counterstaining for 10 minutes. After staining, sample slides were washed in water, dehydrated using ethanol gradient (70%, 90%, 100%), washed three times in HistoClear II (National Diagnostics, HS-202), and mounted in Permount (Fisher Scientific, SP15). Immunostaining analysis for human samples was performed on sections of paraffin-embedded tissues, which included biopsies or resected samples. Staining was performed with antibodies against human p-FLT1/p-VEGFR1 (1:50, Millipore, 07-758), or FLT1/VEGFR1 (2.5 ug/ml, Invitrogen, MA5-32045).

For calculating staining intensity or number of positively stained cells in tumor sections from mice, QuPath 0.3.2 (qupath.github.io/) was used as previously described¹. Image type was set as Brightfield (H-DAB) to count positive (pos.) cells and the cell detection channel was set at Hematoxylin+DAB. The DAB threshold was adjusted and optimized for each antibody staining within Fast Cell Counts feature. To measure different staining intensities, tumor sections were selected and within the Positive Cell Detection feature, detection image was set as Optical density sum. Cell: DAB OD mean was used for scoring each compartment. Each threshold was adjusted on a batch-to-batch basis according to the staining condition to minimize false positive/negative readings. Data was calculated by dividing the number of positive cells by the total area of the tumor section, and Applicants normalized the results to the comparator to present the data as a fold change relative to the control.

For immunostaining analysis of human tumor tissue, immunostaining was scored by pathologists who were blinded to the sample details to assess the expression level of pFLT1 and FLT1 in sections. Staining was assigned as low when staining was scored between 0 and 1 considering the entire field of the tumor section. Staining was assigned as high when staining was scored above 1 up to 4 considering the entire field of the tumor section.

Cell Culture. Brca1- and Bard1-def parental cell lines and their derivatives used in this study were cultured in DMEM media supplemented with 10% FBS and grown at 37° C. in a humidified CO₂ incubator (5% CO₂). All media were supplemented with 100 IU/mL penicillin and 100 μg/mL streptomycin (Life Technologies). To measure viability of Brca1- and Bard1-def sensitive and resistant lines cultured in the presence or absence of talazoparib, 1000 Brca1-def sensitive and resistant cells or 500 of Bard1-def sensitive and resistant cells are plated into each well of a 96-well plate and cultured with growth medium (DMEM supplemented with 10% FBS and Pen-Strep) overnight at 37° C. in a 5% CO₂ incubator. Cells were then treated with 0 to 10,000 nM of talazoparib for 7 days in 0.2 ml of fresh growth medium with changes every three days. On the day of MTS assay, 0.1 ml of medium was removed from each well and 20 μl of CellTiter 96® Aqueous One Solution (Promega G3581) was added to each well. The plates were incubated at 37° C. in a 5% CO₂ incubator for 30 to 120 minutes, and absorbance at 490 nm was obtained with a plate reader. The viability was calculated as a percentage of viable cells in vehicle-treated controls (designated as 100% viability).

RNA Isolation and qRT-PCR. Total RNA (500 ng) was isolated using TRIzol and RNeasy Mini Kit as previously described¹. RNA was then reverse-transcribed to cDNA using a cDNA Synthesis Kit (Applied Biosystems; Thermo Fisher Scientific). qRT-PCR was performed with 10 ng of cDNA per sample using gene-specific primers and SYBR Green PCR master mix (Applied Biosystems; Thermo Fisher Scientific). GAPDH primers were used as an internal control. An Applied Biosystems 7500 Real-Time PCR system (Applied Biosystems; Thermo Fisher Scientific) was used to run all the samples, and data were exported to Excel (Microsoft) for gene expression analysis using the 2^{-ΔΔCt} method. The qRT-PCR primer sequences used in this study are shown below:

mFlt1/mVegfr1:
forward primer:
(SEQ ID NO. 1)
5-TGGCTCTACGACCTTAGACTG-3
reverse primer:
(SEQ ID NO. 2)
5-CAGGTTTGACTTGTCTGAGGTT-3
mGapdh:
forward primer:
(SEQ ID NO. 3)
5-AGGTCGGTGTGAACGGATTTG-3
reverse primer:
(SEQ ID NO. 4)
5-TGTAGACCATGTAGTTGAGGTCA-3

Gene Repression by CRISPR. Applicants knocked down the expression of Flt1/Vegfr1 by the CRISPR/dCas9-KRAB-mediated gene repression method following a previously described method¹. Applicants designed the gRNA sequence (5′-CAGCGCGTAAGGCAAGACCG-3′, SEQ ID NO. 5) using the CRISPR-ERA online tool (crispr-era.stanford.edu). The forward and reverse oligos were designed based on the g RNA sequence and were then annealed and cloned into the BsmBI-digested LentiCRISPRv2-SFFV-KRAB-dCas9¹ following the procedure outlined by Feng Zhang's group². Applicants confirmed the positive clones by PCR using the human U6 forward primer and the reverse oligo of the corresponding gRNA sequence. Applicants produced lentivirus by transfecting the gRNA cloned lentiviral vector into Lenti-X 293T cells line (Takara, cat #632180) using 3rd generation packaging system. Target cells were transduced with viral supernatant (after passing through 0.45-micron syringe filter) and selected after 48 hours post-transduction with puromycin at final concentration of 8 mg/ml. The efficiency of knockdown was tested by RT-PCR using mouse Flt1/Vegfr1 specific primers.

Patient samples. Tumor tissues samples from 10 individuals (pre-PARPi treatment samples) and 11 individuals (PARPi-treated patients) were collected either at the Memorial Sloan Kettering Cancer Center or Emory University School of Medicine in accordance with approved protocols from the institutional review board (IRB), ensuring the protection of patient privacy. The research conducted followed ethical regulations specified by the IRB. Supplementary Tables 1 and 2 provides de-identified information about the germline mutations. Sections were stained with either p-FLT1 and FLT1 antibodies and scored as described in immunohistochemical staining and quantitation section.

Statistical Analysis. Statistical significance was determined by unpaired two-tailed t-test, unpaired Welch's t test, unpaired Mann-Whitney t-test or One-way ANOVA with post-hoc Tukey's test, and log-rank test using Prism 9 software (GraphPad Software). All values were determined as the mean±SEM and P values<0.05 were considered statistically significant.

### REFERENCES CITED AND INCORPORATED BY REFERENCE

1. Venkitaraman, A. R. How do mutations affecting the breast cancer genes BRCA1 and BRCA2 cause cancer susceptibility? DNA Repair (Amst) 81, 102668 (2019).
2. Xu, X., et al. Conditional mutation of Brca1 in mammary epithelial cells results in blunted ductal morphogenesis and tumour formation. Nat Genet 22, 37-43 (1999).
3. Scully, R. & Livingston, D. M. In search of the tumour-suppressor functions of BRCA1 and BRCA2. Nature 408, 429-432 (2000).
4. Groelly, F. J., Fawkes, M., Dagg, R. A., Blackford, A. N. & Tarsounas, M. Targeting DNA damage response pathways in cancer. Nat Rev Cancer 23, 78-94 (2023).
5. Brose, M. S., et al. Cancer risk estimates for BRCA1 mutation carriers identified in a risk evaluation program. J Natl Cancer Inst 94, 1365-1372 (2002).
6. van der Kolk, D. M., et al. Penetrance of breast cancer, ovarian cancer and contralateral breast cancer in BRCA1 and BRCA2 families: high cancer incidence at older age. Breast Cancer Res Treat 124, 643-651 (2010).
7. Hall, J. M., et al. Linkage of early-onset familial breast cancer to chromosome 17q21. Science 250, 1684-1689 (1990).
8. Miki, Y., et al. A strong candidate for the breast and ovarian cancer susceptibility gene BRCA1. Science 266, 66-71 (1994).
9. Ford, D., Easton, D. F., Bishop, D. T., Narod, S. A. & Goldgar, D. E. Risks of cancer in BRCA1-mutation carriers. Breast Cancer Linkage Consortium. Lancet 343, 692-695 (1994).
10. Ford, D., et al. Genetic heterogeneity and penetrance analysis of the BRCA1 and BRCA2 genes in breast cancer families. The Breast Cancer Linkage Consortium. Am J Hum Genet 62, 676-689 (1998).
11. Nik-Zainal, S., et al. Landscape of somatic mutations in 560 breast cancer whole-genome sequences. Nature 534, 47-54 (2016).
12. Vidula, N., et al. Identification of Somatically Acquired BRCA1/2 Mutations by cfDNA Analysis in Patients with Metastatic Breast Cancer. Clin Cancer Res 26, 4852-4862 (2020).
13. Foulkes, W. D., et al. Estrogen receptor status in BRCA1- and BRCA2-related breast cancer: the influence of age, grade, and histological type. Clin Cancer Res 10, 2029-2034 (2004).
14. Comen, E., et al. Relative contributions of BRCA1 and BRCA2 mutations to “triple-negative” breast cancer in Ashkenazi Women. Breast Cancer Res Treat 129, 185-190 (2011).
15. Farmer, H., et al. Targeting the DNA repair defect in BRCA mutant cells as a therapeutic strategy. Nature 434, 917-921 (2005).
16. Bryant, H. E., et al. Specific killing of BRCA2-deficient tumours with inhibitors of poly(ADP-ribose) polymerase. Nature 434, 913-917 (2005).
17. D'Andrea, A. D. Mechanisms of PARP inhibitor sensitivity and resistance. DNA Repair (Amst) 71, 172-176 (2018).
18. Comen, E. A. & Robson, M. Inhibition of poly (ADP)-ribose polymerase as a therapeutic strategy for breast cancer. Oncology (Williston Park) 24, 55-62 (2010).
19. Acharyya, S., et al. A CXCL1 paracrine network links cancer chemoresistance and metastasis. Cell 150, 165-178 (2012).
20. Waks, A. G., et al. Reversion and non-reversion mechanisms of resistance to PARP inhibitor or platinum chemotherapy in BRCA1/2-mutant metastatic breast cancer. Ann Oncol 31, 590-598 (2020).
21. Rottenberg, S., et al. High sensitivity of BRCA1-deficient mammary tumors to the PARP inhibitor AZD2281 alone and in combination with platinum drugs. Proc Natl Acad Sci USA 105, 17079-17084 (2008).
22. Henneman, L., et al. Selective resistance to the PARP inhibitor olaparib in a mouse model for BRCA1-deficient metaplastic breast cancer. Proc Natl Acad Sci USA 112, 8409-8414 (2015).
23. Shakya, R., et al. The basal-like mammary carcinomas induced by Brca1 or Bard1 inactivation implicate the BRCA1/BARD1 heterodimer in tumor suppression. Proc Natl Acad Sci USA 105, 7040-7045 (2008).
24. Wu, L. C., et al. Identification of a RING protein that can interact in vivo with the BRCA1 gene product. Nat Genet 14, 430-440 (1996).
25. Lim, B. W. X., et al. Somatic inactivation of breast cancer predisposition genes in tumors associated with pathogenic germline variants. J Natl Cancer Inst 115, 181-189 (2023).
26. Drost, R., et al. BRCA1185delAG tumors may acquire therapy resistance through expression of RING-less BRCA1. J Clin Invest 126, 2903-2918 (2016).
27. Powell, S. N. BRCA1 loses the ring but lords over resistance. J Clin Invest 126, 2802-2804 (2016).
28. Wang, Y., et al. RING domain-deficient BRCA1 promotes PARP inhibitor and platinum resistance. J Clin Invest 126, 3145-3157 (2016).
29. McCabe, N., et al. Deficiency in the repair of DNA damage by homologous recombination and sensitivity to poly(ADP-ribose) polymerase inhibition. Cancer Res 66, 8109-8115 (2006).
30. Dias, M. P., Moser, S. C., Ganesan, S. & Jonkers, J. Understanding and overcoming resistance to PARP inhibitors in cancer therapy. Nat Rev Clin Oncol 18, 773-791 (2021).
31. Moreno-Lama, L., et al. Coordinated signals from PARP-1 and PARP-2 are required to establish a proper T cell immune response to breast tumors in mice. Oncogene 39, 2835-2843 (2020).
32. Ding, L., et al. PARP Inhibition Elicits STING-Dependent Antitumor Immunity in Brca1-Deficient Ovarian Cancer. Cell Rep 25, 2972-2980 e2975 (2018).
33. Galindo-Campos, M. A., et al. Coordinated signals from the DNA repair enzymes PARP-1 and PARP-2 promotes B-cell development and function. Cell Death Differ 26, 2667-2681 (2019).
34. Shen, J., et al. PARPi Triggers the STING-Dependent Immune Response and Enhances the Therapeutic Efficacy of Immune Checkpoint Blockade Independent of BRCAness. Cancer Res 79, 311-319 (2019).
35. Wang, Q., et al. STING agonism reprograms tumor-associated macrophages and overcomes resistance to PARP inhibition in BRCA1-deficient models of breast cancer. Nat Commun 13, 3022 (2022).
36. Shakri, A. R., et al. Aberrant Zip14 expression in muscle is associated with cachexia in a Bard1-deficient mouse model of breast cancer metastasis. Cancer Med 9, 6766-6775 (2020).
37. Zhang, Y. & Brekken, R. A. Direct and indirect regulation of the tumor immune microenvironment by VEGF. J Leukoc Biol 111, 1269-1286 (2022).
38. Ferrara, N., Gerber, H. P. & LeCouter, J. The biology of VEGF and its receptors. Nat Med 9, 669-676 (2003).
39. Shibuya, M. Structure and function of VEGF/VEGF-receptor system involved in angiogenesis. Cell Struct Funct 26, 25-35 (2001).
40. Le Saux, O., et al. Poly(ADP-ribose) polymerase inhibitors in combination with anti-angiogenic agents for the treatment of advanced ovarian cancer. Future Oncol 17, 2291-2304 (2021).
41. Biswas, A. K., et al. Targeting S100A9-ALDH1A1-Retinoic Acid Signaling to Suppress Brain Relapse in EGFR-Mutant Lung Cancer. Cancer Discov 12, 1002-1021 (2022).
42. Tyler, T. Axitinib: newly approved for renal cell carcinoma. J Adv Pract Oncol 3, 333-335 (2012).
43. Tchaikovski, V., Fellbrich, G. & Waltenberger, J. The molecular basis of VEGFR-1 signal transduction pathways in primary human monocytes. Arterioscler Thromb Vasc Biol 28, 322-328 (2008).
44. Selvaraj, S. K., et al. Mechanism of monocyte activation and expression of proinflammatory cytochemokines by placenta growth factor. Blood 102, 1515-1524 (2003).
45. Bellik, L., Vinci, M. C., Filippi, S., Ledda, F. & Parenti, A. Intracellular pathways triggered by the selective FLT-1-agonist placental growth factor in vascular smooth muscle cells exposed to hypoxia. Br J Pharmacol 146, 568-575 (2005).
46. Bartoli, M., et al. Vascular endothelial growth factor activates STAT proteins in aortic endothelial cells. J Biol Chem 275, 33189-33192 (2000).
47. Chen, S. H., et al. Activated STAT3 is a mediator and biomarker of VEGF endothelial activation. Cancer Biol Ther 7, 1994-2003 (2008).
48. Tung, N. & Garber, J. E. PARP inhibition in breast cancer: progress made and future hopes. NPJ Breast Cancer 8, 47 (2022).
49. Robson, M., et al. Olaparib for Metastatic Breast Cancer in Patients with a Germline BRCA Mutation. N Engl J Med 377, 523-533 (2017).
50. Fong, G. H., Rossant, J., Gertsenstein, M. & Breitman, M. L. Role of the Flt-1 receptor tyrosine kinase in regulating the assembly of vascular endothelium. Nature 376, 66-70 (1995).
51. Fong, G. H., Zhang, L., Bryce, D. M. & Peng, J. Increased hemangioblast commitment, not vascular disorganization, is the primary defect in flt-1 knock-out mice. Development 126, 3015-3025 (1999).
52. Barleon, B., et al. Migration of human monocytes in response to vascular endothelial growth factor (VEGF) is mediated via the VEGF receptor flt-1. Blood 87, 3336-3343 (1996).
53. Clauss, M., et al. The vascular endothelial growth factor receptor Flt-1 mediates biological activities. Implications for a functional role of placenta growth factor in monocyte activation and chemotaxis. J Biol Chem 271, 17629-17634 (1996).
54. Hiratsuka, S., Minowa, O., Kuno, J., Noda, T. & Shibuya, M. Flt-1 lacking the tyrosine kinase domain is sufficient for normal development and angiogenesis in mice. Proc Natl Acad Sci USA 95, 9349-9354 (1998).
55. Hattori, K., et al. Placental growth factor reconstitutes hematopoiesis by recruiting VEGFR1 (+) stem cells from bone-marrow microenvironment. Nat Med 8, 841-849 (2002).
56. Kaplan, R. N., et al. VEGFR1-positive haematopoietic bone marrow progenitors initiate the pre-metastatic niche. Nature 438, 820-827 (2005).
57. Qian, B. Z., et al. FLT1 signaling in metastasis-associated macrophages activates an inflammatory signature that promotes breast cancer metastasis. J Exp Med 212, 1433-1448 (2015).
58. Yao, J., et al. Expression of a functional VEGFR-1 in tumor cells is a major determinant of anti-PIGF antibodies efficacy. Proc Natl Acad Sci USA 108, 11590-11595 (2011).
59. Wu, Y., et al. The vascular endothelial growth factor receptor (VEGFR-1) supports growth and survival of human breast carcinoma. Int J Cancer 119, 1519-1529 (2006).
60. Lesslie, D. P., et al. Vascular endothelial growth factor receptor-1 mediates migration of human colorectal carcinoma cells by activation of Src family kinases. Br J Cancer 94, 1710-1717 (2006).
61. Wey, J. S., et al. Vascular endothelial growth factor receptor-1 promotes migration and invasion in pancreatic carcinoma cell lines. Cancer 104, 427-438 (2005).
62. Frank, N. Y., et al. VEGFR-1 expressed by malignant melanoma-initiating cells is required for tumor growth. Cancer Res 71, 1474-1485 (2011).
63. Sopo, M., et al. Expression profiles of VEGF-A, VEGF-D and VEGFR1 are higher in distant metastases than in matched primary high grade epithelial ovarian cancer. BMC Cancer 19, 584 (2019).
64. Tentori, L., et al. Poly(ADP-ribose) polymerase (PARP) inhibition or PARP-1 gene deletion reduces angiogenesis. Eur J Cancer 43, 2124-2133 (2007).
65. Bizzaro, F., et al. VEGF pathway inhibition potentiates PARP inhibitor efficacy in ovarian cancer independent of BRCA status. J Hematol Oncol 14, 186 (2021).
66. Boocock, C. A., et al. Expression of vascular endothelial growth factor and its receptors flt and KDR in ovarian carcinoma. J Natl Cancer Inst 87, 506-516 (1995).
67. Alvarez Secord, A., O'Malley, D. M., Sood, A. K., Westin, S. N. & Liu, J. F. Rationale for combination PARP inhibitor and antiangiogenic treatment in advanced epithelial ovarian cancer: A review. Gynecol Oncol 162, 482-495 (2021).
68. Martinsson-Niskanen, T., et al. Monoclonal antibody TB-403: a first-in-human, Phase I, double-blind, dose escalation study directed against placental growth factor in healthy male subjects. Clin Ther 33, 1142-1149 (2011).
69. Lassen, U., et al. A phase I, dose-escalation study of TB-403, a monoclonal antibody directed against PIGF, in patients with advanced solid tumours. Br J Cancer 106, 678-684 (2012).
70. Saulnier-Sholler, G., et al. A Phase I Trial of TB-403 in Relapsed Medulloblastoma, Neuroblastoma, Ewing Sarcoma, and Alveolar Rhabdomyosarcoma. Clin Cancer Res 28, 3950-3957 (2022).
71. Biswas, A. K., et al. Targeting S100A9-ALDH1A1-Retinoic Acid Signaling to Suppress Brain Relapse in EGFR-Mutant Lung Cancer. Cancer Discov 12, 1002-1021 (2022).
72. Sanjana, N. E., Shalem, O. & Zhang, F. Improved vectors and genome-wide libraries for CRISPR screening. Nat Methods 11, 783-784 (2014).

### Example 2—FLT1 Activation in Cancer Cells Promotes PARP-Inhibitor Resistance in Breast Cancer (Tai Y, Chow A, Han S, et al. FLT1 Activation in Cancer Cells Promotes PARP-Inhibitor Resistance in Breast Cancer. EMBO Mol Med. 2024; 16 (8): 1957-1980)

Acquired resistance to PARP inhibitors (PARPi) remains a treatment challenge for BRCA1/2-mutant breast cancer that drastically shortens patient survival. Although several resistance mechanisms have been identified, none have been successfully targeted in the clinic. Using new PARPi-resistance models of Brca1- and Bard1-mutant breast cancer generated in-vivo, Applicants identified FLT1 (VEGFR1) as a driver of resistance. Unlike the known role of VEGF signaling in angiogenesis, Applicants demonstrate a novel, non-canonical role for FLT1 signaling that protects cancer cells from PARPi in-vivo through a combination of cell-intrinsic and cell-extrinsic pathways. Applicants demonstrate that FLT1 blockade suppresses AKT activation, increases tumor infiltration of CD8⁺ T cells, and causes dramatic regression of PARPi-resistant breast tumors in a T-cell-dependent manner. Moreover, PARPi-resistant tumor cells can be readily re-sensitized to PARPi by targeting Flt1 either genetically (Flt1-suppression) or pharmacologically (axitinib). Importantly, a retrospective series of breast cancer patients treated with PARPi demonstrated shorter progression-free survival in cases with FLT1 activation at pre-treatment. The study therefore identifies FLT1 as a potential therapeutic target in PARPi-resistant, BRCA1/2-mutant breast cancer.

### Synopsis

PARP inhibitor (PARPi) resistance is a major treatment challenge that dramatically shortens patient survival. Using new mouse models of PARPi response and recurrence, Applicants identified FLT1 as a potential biomarker and therapeutic target for reversing PARPi resistance in BRCA-mutant breast cancer.
New mouse models were developed that recapitulate the PARPi response and recurrence observed in patients.
A novel PARPi-adaptive resistance mechanism driven by the PGF-FLT1-AKT pathway was identified.
FLT1 signaling protected the cells from PARPi-induced death by activating AKT pro-survival pathways and by dampening the cytotoxic immune response.
Blocking FLT1 signaling, either genetically or pharmacologically using axitinib, re-sensitized PARPi-resistant tumors to PARPi treatment in mice.
High FLT1 activation in tumor cells at pre-treatment significantly correlated with shorter progression-free survival on PARPi in patients with breast cancer.

### Problems

Resistance to PARP inhibitors (PARPi) remains a major treatment challenge in BRCA1/2-mutant breast cancer. Although several resistance mechanisms involving the DNA damage response (DDR) pathway have been identified, none have been successfully targeted in the clinic.

### Results

In this study, Applicants generated new in-vivo treatment models of Brca1- and Bard1-mutant breast cancer that recapitulate the striking response to PARPi, acquired resistance, and recurrence that is observed in patients with breast cancer. Distinct from previously defined resistance mechanisms, Applicants identified a novel PGF-FLT1-AKT signaling pathway of adaptive resistance to PARPi using these models. Mechanistically, activated FLT1 in PARPi-resistant cancer cells promotes cell survival through AKT activation and reduced infiltration of CD8⁺ T cells in tumors. Importantly, genetic and pharmacological inhibition of FLT1 using axitinib re-sensitizes resistant cancer cells to PARPi. Consistent with the preclinical studies, FLT1 activation in tumor cells at pre-treatment significantly correlates with shorter progression-free survival on PARPi in patients with breast cancer.

### Impact

This study, for the first time, identified a potentially actionable PGF-FLT1-AKT axis that mediates PARPi resistance in breast cancer. Of significance, FLT1 expression in cancer cells can be used as a biomarker to stratify patients who might benefit from combination treatment with axitinib and PARPi. Since axitinib is already FDA-approved for renal cancer treatment, it can be re-purposed to enhance the efficacy and durability of PARPi treatment in breast cancer.

### Introduction

The tumor suppressor genes breast cancer 1 (BRCA1) and breast cancer 2 (BRCA2) maintain cellular genome integrity through various processes, including homologous recombination (HR)-mediated repair of DNA breaks (Groelly et al, 2023; Scully and Livingston, 2000; Venkitaraman, 2019; Xu et al, 1999). Individuals harboring heterozygous germline mutations in either BRCA1 or BRCA2 (collectively denoted as “BRCA1/2” hereafter) display heightened susceptibility to certain malignancies, especially breast and ovarian cancers (Ford et al, 1994; Ford et al, 1998) that arise upon loss of the remaining wild-type BRCA1/2 alleles and the onset of extensive genome instability (Brose et al, 2002; Hall et al, 1990; Miki et al, 1994; van der Kolk et al, 2010). In addition to the germline mutations implicated in familial breast cancer, somatic BRCA1/2 mutations are also detected in some sporadic cases of breast cancer (Nik-Zainal et al, 2016; Vidula et al, 2020). Most BRCA1-mutated breast tumors, and a subset of BRCA2-mutated tumors, present as triple-negative breast cancer (TNBC), which is associated with a poor prognosis and high likelihood of recurrence (Comen et al, 2011; Foulkes et al, 2004).

In cells that experience genotoxic stress, the poly(ADP-ribose) polymerases PARP1 and PARP2 promote the DNA damage response (DDR) by recognizing DNA breaks and by PARylating a variety of DDR factors, including those involved in single-strand DNA repair (Comen and Robson, 2010; D'Andrea, 2018; Dias et al, 2021). Since BRCA1/2-mutant tumor cells are deficient in HR-mediated DNA repair, they are especially reliant on PARP1/2 for their survival (Bryant et al, 2005; Farmer et al, 2005; Venkitaraman, 2019). The discovery of synthetic lethality between PARP enzymatic inhibition and BRCA1/2 mutations ultimately led to the rapid clinical translation of PARP inhibitors (PARPi) for the treatment of BRCA1/2-mutant breast cancers (Bryant et al, 2005; Farmer et al, 2005). Among patients with BRCA1/2-mutant breast cancer, dramatic initial responses to PARPi drugs, such as olaparib and talazoparib, led to their FDA approval as monotherapy (Comen and Robson, 2010; D'Andrea, 2018; Litton et al, 2018; Robson et al, 2017). However, responses were short-lived and typically resulted in lethal recurrences, thus prompting the search for mechanisms of PARPi resistance.

Early work established that PARPi resistance can develop in a subset of breast cancer patients by reversion mutations in the BRCA1/2 genes that restore HR function and thereby abrogate cellular dependence on PARP1/2 (Barber et al, 2013; Lin et al, 2019; Pettitt et al, 2020; Tobalina et al, 2021; Waks et al, 2020). In addition, systematic analyses of PARPi resistance mechanisms using BRCA1/2-mutant cells, both in culture and in mice, have identified alterations in a variety of other DDR genes that can potentially promote PARPi resistance by restoring HR activity (Berti et al, 2020; Bhin et al, 2023; Cruz et al, 2018; D'Andrea, 2018; Dias et al, 2021; Drost et al, 2016; Henneman et al, 2015; Hobbs et al, 2021; McCabe et al, 2006; Powell, 2016; Rottenberg et al, 2008; Wang et al, 2016). Indeed, some of the DDR genes implicated in these experimental studies (e.g., TP53BP1 and MRE11A) have also been observed in PARPi-resistant tumors from patients with BRCA1/2-mutant breast cancer (Waks et al, 2020). Despite this progress, circumventing PARPi resistance is not currently feasible in the clinic.

To identify alternative mechanisms of PARPi resistance, Applicants generated orthotopic allografts using tumor cells derived from genetically engineered mouse models (GEMMs) of Brca1- or Bard1-deficient breast cancer (Shakya et al, 2008). Most BRCA1 functions, including HR-mediated DNA repair, are executed by the BRCA1/BARD1 heterodimer, a nuclear complex formed by BRCA1 and BRCA1-associated RING domain 1 (BARD1) proteins (Lim et al, 2023; Wu et al, 1996). Genetic inactivation of either Brca1 or Bard1 in mammary epithelial cells leads to the development of triple-negative carcinomas that are indistinguishable in latency, frequency, cytogenetic features, and histopathology (Shakya et al, 2008). Applicants chose to study PARPi resistance using Brca1- and Bard1-deficient allograft models for two main reasons. First, these models recapitulate the typical clinical course of PARPi therapy, which is characterized by a striking initial response to treatment, the subsequent development of resistance, and finally, cancer progression (D'Andrea, 2018; Dias et al, 2021; Tung and Garber, 2022). Second, many experimental studies of PARPi resistance mechanisms have been conducted using cell lines or subcutaneous tumors (xenografts) in immunocompromised mice. Since PARPi treatment is known to impact both innate and adaptive immunity (Ding et al, 2018; Galindo-Campos et al, 2019; Moreno-Lama et al, 2020; Shen et al, 2019; Wang et al, 2022), Applicants sought to model the clinical setting and physiological context in which PARPi resistance develops using the new Brca1- and Bard1-deficient treatment models.

Analogous to breast cancer patients with BRCA1 mutations, mice bearing Brca1- and Bard1-deficient tumors display remarkable initial responses to PARPi that are inevitably followed by disease progression. As expected, the tumors that progress are no longer sensitive to PARPi treatment in-vivo. Surprisingly, however, the PARPi-resistant tumor cells retain their sensitivity to PARPi in vitro, suggesting that adaptive mechanisms operating in the tumor microenvironment protect these PARPi-sensitive cells from PARP inhibition in-vivo. In contrast to resistance mechanisms that restore the DDR functions of BRCA1, the study identified FLT1 (VEGFR1) signaling as a novel driver of in-vivo PARPi resistance that can be therapeutically targeted. Applicants show that FLT1 is activated in the tumor cells of PARPi-resistant tumors from Brca1- and Bard1-deficient models as well as from breast cancer patients. Mechanistically, FLT1 signaling protects these cells from PARPi-induced death by activating AKT pro-survival pathways and by dampening the cytotoxic immune response. Moreover, patients with BRCA1/2- or PALB2 (partner and localizer of BRCA2)-mutant breast tumors that display heightened FLT1 expression in their tumor cells at pre-treatment are at high risk for progression on PARPi. Importantly, Applicants observed that blocking FLT1 signaling, either genetically or pharmacologically, re-sensitizes PARPi-resistant tumors to PARPi treatment. Thus, by circumventing the development of PARPi resistance in breast cancer patients, FLT1 blockade may serve to unleash the full clinical potential of PARPi therapy.

### Results

### Generation of In-Vivo PARPi-Response-and-Progression Models of Brca1- and Bard1-Deficient Breast Cancer

To study mechanisms of PARPi resistance in-vivo, Applicants isolated Brca1- or Bard1-deficient breast tumor cells (hereafter referred to as “Brca1-def” and “Bard1-def”) derived from Brca1- or Bard1-conditionally deleted mice (Shakya et al, 2008) and injected these cells in the mammary glands of syngeneic B6/129F1 mice (FIG. 1). Tumor-bearing mice were randomized and treated five days/week with either vehicle (referred to as “Veh” in the figures) or the PARPi talazoparib (referred to as “Tal” in the figures), an FDA-approved PARPi in human breast cancer, at a dose of 0.3 mg/kg body weight/day starting at either 14 days (Brca1-def) or 7 days (Bard1-def) post tumor-cell injection (see schematic in FIGS. 1A,D). Tumor size was measured weekly, and tumors were collected either when they reached the size limit or when mice developed a body-conditioning score (BCS) of 2 or less, following the guidelines for euthanasia. Although PARPi treatment suppressed tumor growth for several weeks, tumors began to progress and eventually became refractory to the treatment in all treated Brca1-def mice (FIG. 1B). Analogous to the Brca1-def model, Bard1-def tumors also responded initially to PARPi but soon progressed (FIG. 1E). While tumor growth in Brca1-def models was monitored for 13 weeks post tumor-cell injection, Bard1-def models could only be monitored for 5 weeks due to the onset of cachexia, as described in the previous studies (Shakri et al, 2020). Tumor cells that were are refractory to PARPi (abbreviated as “PARPi-Res” in the figure) from both PARPi-treated Brca1-def and Bard1-def tumors were then isolated, re-injected into the mammary glands of new B6/129F1 recipient mice (FIGS. 1C,F) and treated as described above. Notably, in contrast to the tumors generated from parental PARPi-sensitive tumor cells (i.e., PARPi-sensitive, abbreviated as “PARPi-Sen” in FIGS. 1B,E), tumors derived from PARPi-treated mice (PARPi-Res tumors) failed to respond in-vivo to PARPi starting from the onset of treatment (FIGS. 1C,F), confirming the acquisition of drug resistance. Surprisingly, while the difference in response to talazoparib between the PARPi-sensitive and PARPi-resistant tumor lines was striking in-vivo (FIG. 1B compared to 1C and 1E compared to 1F), only a modest difference was observed in vitro (FIGS. 8A,B), albeit to different extents between the two cell lines. These results suggest that the tumor microenvironment and physiological context may be critical for the development of PARPi resistance in these models.

PARPi-Resistant Tumors Show Increased VEGFR2 and PGF Expression but Only Modest Sensitization to PARPi Upon VEGFR2 Depletion

Since PARPi resistance in the Brca1-def and Bard1-def models was more faithfully recapitulated in-vivo than in vitro (FIG. 1; FIG. 8), Applicants hypothesized that signals from the tumor microenvironment are important for mediating PARPi resistance. To identify mechanisms of PARPi resistance, Applicants compared the tumor microenvironments of PARPi-sensitive and -resistant tumors by immunohistochemical staining (FIGS. 2A-H; FIGS. 9A,B). The PARPi-resistant tumors showed a significant increase in CD31⁺ endothelial cells (FIGS. 2A,B), indicating a greater amount of angiogenesis in PARPi-resistant tumors compared to PARPi-sensitive tumors. Analysis of the major immune-cell populations revealed a marked reduction in the number of CD8⁺ T cells and increased numbers of both CD4⁺ helper T cells and CD11C⁺ dendritic cells in PARPi-resistant compared to PARPi-sensitive tumors (FIGS. 9A,B). However, B cells and macrophages showed no consistent differences between the sensitive and resistant tumors in the Brca1-def and Bard1-def models (FIGS. 9A,B). These results indicate that changes in angiogenesis and immune-cell composition may underlie PARPi resistance in BRCA1-mutant breast tumors.

Since the vascular endothelial growth factor (VEGF) signaling pathway regulates both angiogenesis and the immune response (Ferrara et al, 2003; Zhang and Brekken, 2022), Applicants performed immunohistochemical staining to determine whether the VEGF pathway is activated in the PARPi-resistant tumors. Among the VEGF family of ligands, VEGFA expression was modestly increased while placental growth factor (PGF) was markedly increased (Brca1-def: 5.9-fold, p=0.0079 and Bard1-def: 22.5-fold, p=0.0159) in the PARPi-resistant tumors (FIGS. 2C-F). These results indicate that signaling through the VEGF family of ligands might be important for PARPi resistance.

VEGFR2 (also known as KDR) is a key receptor in vascular endothelial cells that recognizes the VEGF family of ligands and serves as a major signal transducer for angiogenesis (Shibuya, 2001). Consistent with an increase in CD31⁺ endothelial cells (FIGS. 2A,B), the number of VEGFR2-expressing endothelial cells also increased significantly in the PARPi-resistant tumors compared to PARPi-sensitive tumors (FIGS. 2G,H). Pathological evaluation of the immunohistochemistry revealed that VEGFR2 is expressed primarily in the endothelial cells (as expected) but not in the tumor cells (FIG. 2G). Since PARPis can synergize with anti-angiogenic agents in ovarian cancer (Le Saux et al, 2021), Applicants examined whether blocking pro-angiogenic VEGFR2 signaling could sensitize PARPi-resistant breast tumors to talazoparib. To this end, Applicants implanted PARPi-resistant tumor cells from the Brca1-def and Bard1-def models in the mammary glands of new recipient B6/129F1 mice and treated them with either VEGFR2 antibody (200 μg/mouse three times a week) or isotype control antibody in combination with vehicle or talazoparib (schematic in FIG. 2I). VEGFR2 blockade in combination with talazoparib reduced the number of CD31⁺ endothelial cells in PARPi-treated tumors in both the Brca1-def and Bard1-def models (EV2C), which confirmed the efficacy of the VEGFR2 antibody treatment. Interestingly, despite reduced angiogenesis, VEGFR2 inhibition in combination with talazoparib only modestly reduced tumor growth in the Brca1-def and Bard1-def models (FIGS. 2J,K; FIG. 9D), suggesting that other mechanisms are important for driving in-vivo PARPi resistance.

### FLT1 (VEGFR1) Activation in Tumor Cells Promotes PARPi Resistance in Breast Cancer

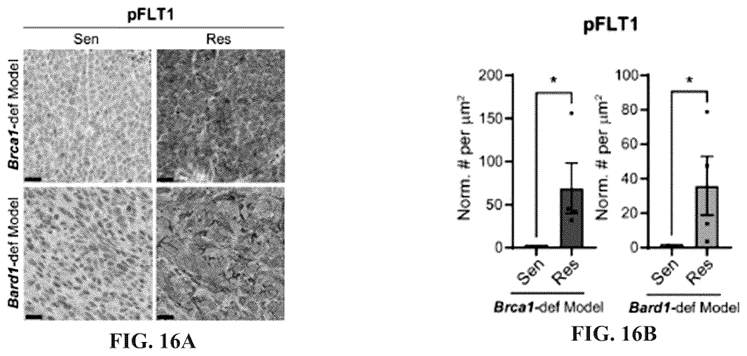

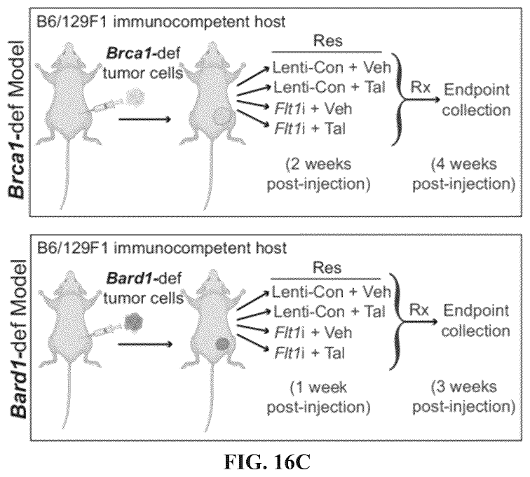

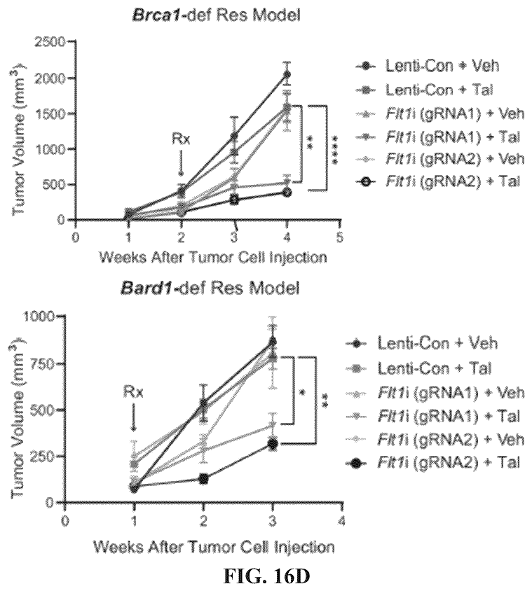

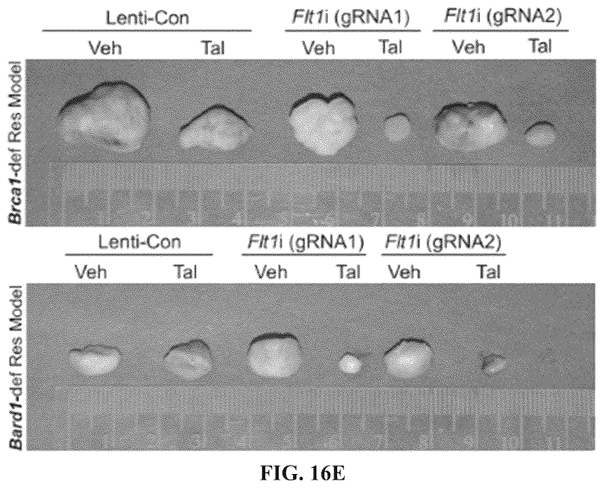

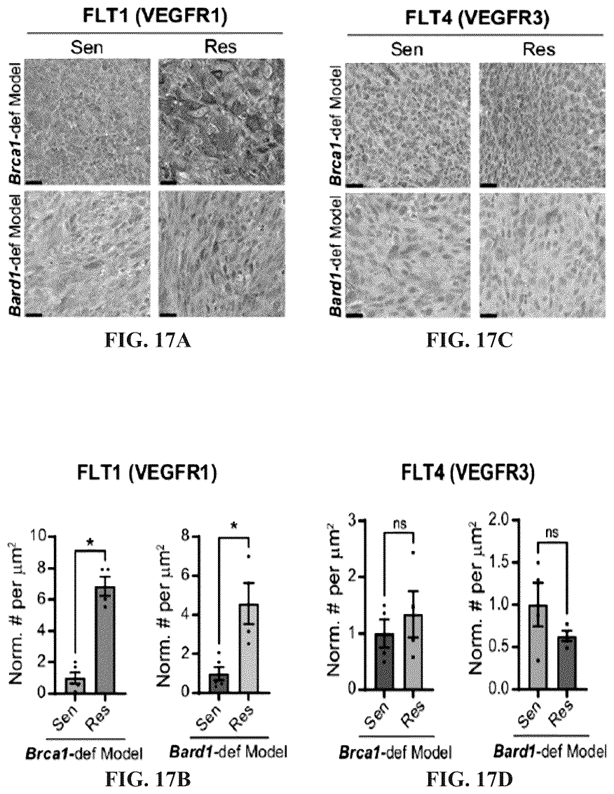

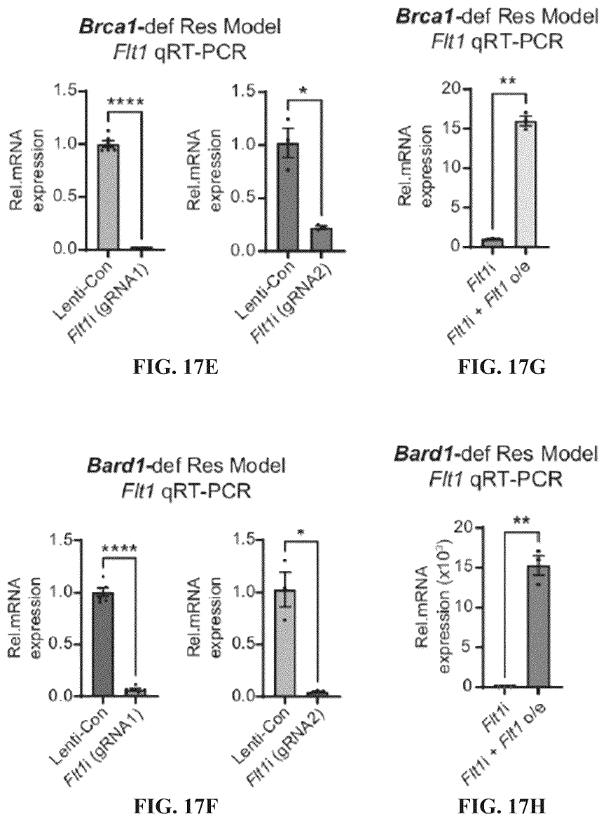

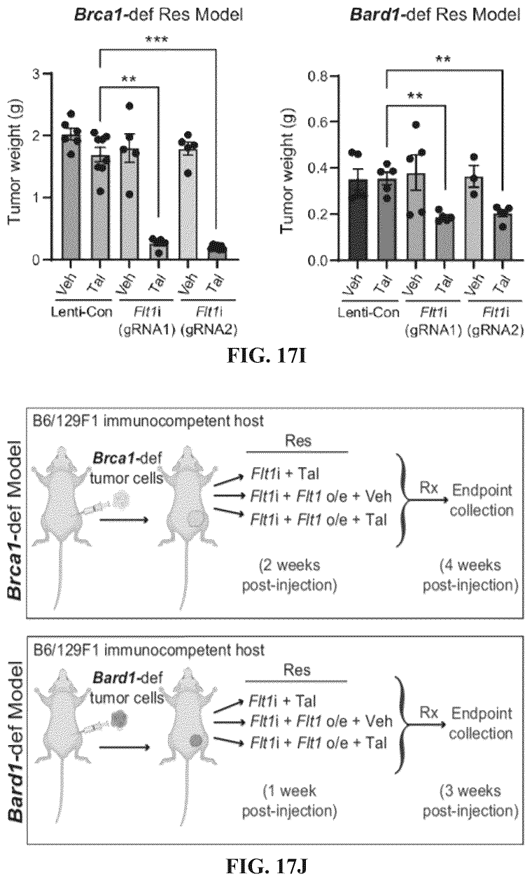

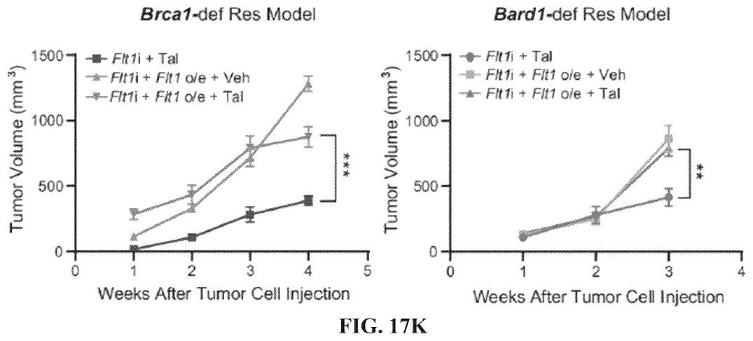

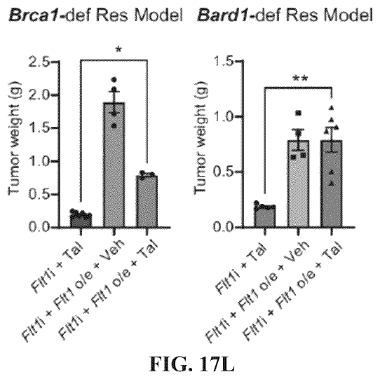

The high expression of PGF in PARPi-resistant tumors (FIGS. 2E,F) prompted Applicants to investigate whether the PGF receptor FLT1 (also known as VEGFR1) contributes to PARPi resistance. Indeed, by immunohistochemical analysis, Applicants observed a striking increase in levels of both phosphorylated FLT1 (Tyr1213; referred to as “pFLT1” hereafter) and total FLT1 in tumor cells from PARPi-resistant tumors compared to PARPi-sensitive tumors (FIGS. 16A,B; 17A,B). In contrast, no significant changes in FLT4 (also known as VEGFR3) expression were observed (FIGS. 17C,D). Based on these observations, Applicants hypothesized that the increased PGF levels in the tumor microenvironment induced by PARPi treatment may, in turn, promote PARPi resistance by activating FLT1 signaling in the tumor cells. To test this hypothesis, Applicants performed loss- and gain-of-function experiments to determine whether genetic repression of Flt1 in PARPi-resistant tumor cells impacts tumor growth in the presence of PARPi, and whether this effect is rescued by FLT1 re-expression. Applicants first engineered lentiviruses to deplete Flt1 expression (“Flt1i”) in the tumor cells by CRISPR-mediated gene repression (CRISPRi) using two independent guide RNAs (“gRNA1” or “gRNA2”), as described previously (Biswas et al, 2022) (FIGS. 17E,F). For gain-of-function experiments, Applicants re-expressed FLT1 in FLT1-repressed cancer cells (“Flt1i+Flt1 o/e”, FIGS. 17G,H). Next, PARPi-resistant Brca1-def and Bard1-def tumor cells, transduced with either control lentivirus (“Lenti-Con”), Flt1i lentivirus, or Flt1i lentivirus plus Flt1 o/e lentivirus, were injected orthotopically in the mammary glands of syngeneic B6/129F1 mice. As illustrated in FIG. 16C; FIG. 17J, mice were randomized and treated five days/week with either vehicle or talazoparib (0.3 mg/kg body weight/day). An analysis of tumor growth and weight at endpoint revealed that Flt1 repression in tumor cells re-sensitizes PARPi-resistant tumors to talazoparib (FIGS. 16D,E; FIG. 17I), which can be rescued by FLT1 re-expression (FIGS. 17K,L).

### a Pan-VEGFR Antagonist (Axitinib) Re-Sensitizes PARPi-Resistant Breast Tumors to PARPi Treatment

Applicants next tested whether PARPi-resistant tumors could also be re-sensitized by blocking FLT1 signaling pharmacologically using the pan-VEGFR inhibitor axitinib, an FDA-approved drug for treating metastatic renal-cell carcinoma patients (Tyler, 2012). PARPi-resistant Brca1-def and Bard1-def tumor cells were injected orthotopically into the mammary gland of syngeneic B6/129F1 mice (see schematic in FIGS. 4A,D). Mice were randomized and treated five days/week with either vehicle, talazoparib (0.3 mg/kg body weight/day), axitinib (30 mg/kg body weight/day), or talazoparib-plus-axitinib (0.3 mg/kg body weight/day and 30 mg/kg body weight/day, respectively). In contrast to the single-treatment groups, Applicants observed a striking reduction in the tumor burden of mice treated with both talazoparib and axitinib (FIGS. 4B,C,E,F; FIGS. 11A,B). None of the treatments led to overt toxicities, and stable body weight was maintained by all mice for the duration of the studies (≥3 weeks) (FIGS. 11C,D). Thus, the combination of PARPi and FLT1 blockade is highly effective at suppressing PARPi resistance in-vivo and inhibiting the growth of tumors lacking BRCA1 function.

### FLT1 Activation Induces Pro-Survival AKT Signaling in PARPi-Resistant Tumor Cells

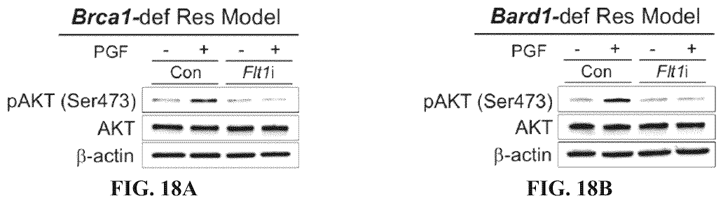

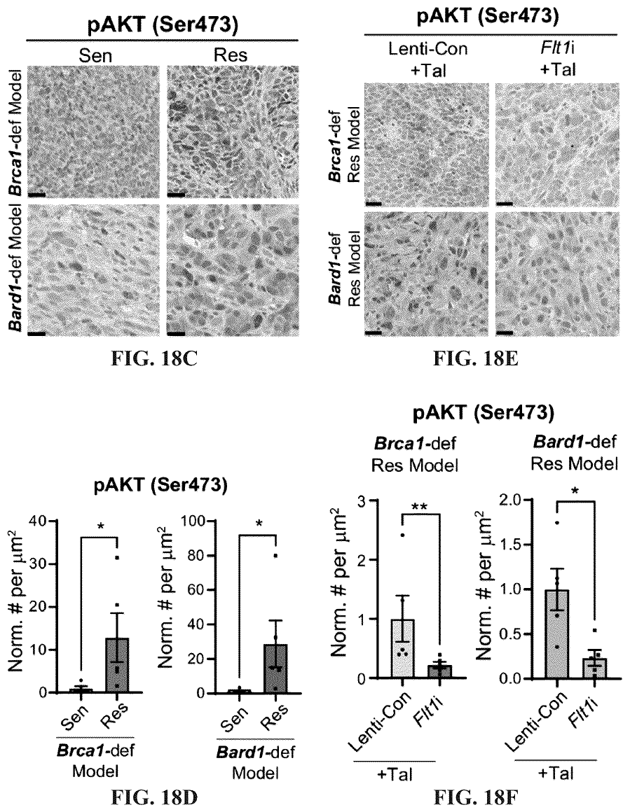

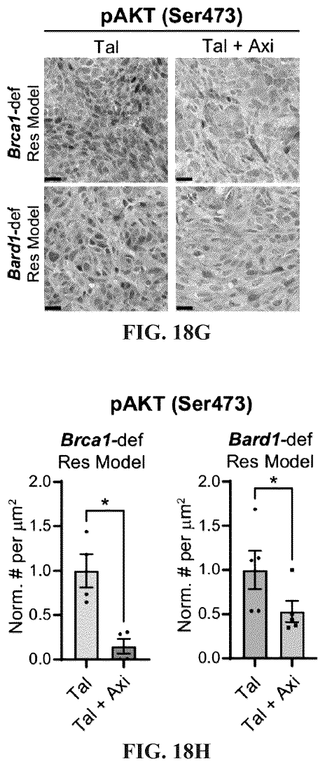

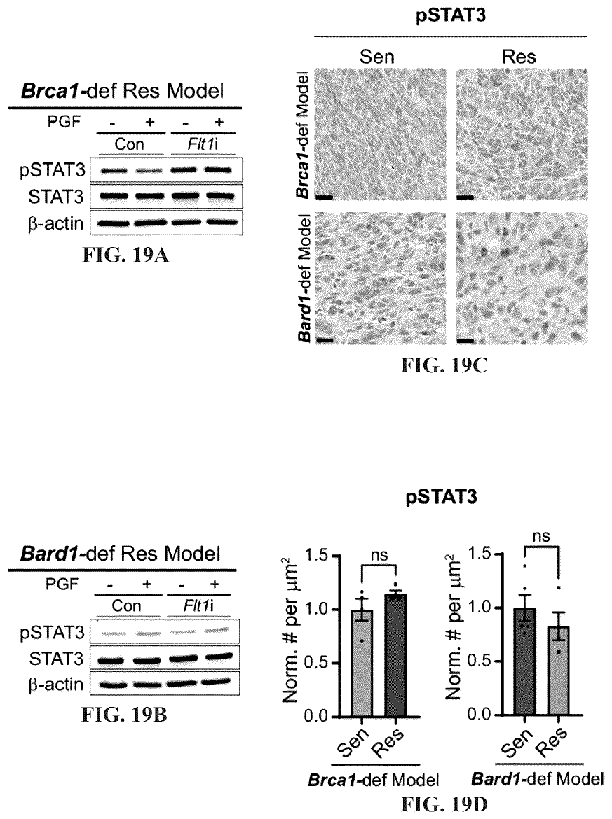

To investigate how FLT1 activation in breast tumor cells counteracts PARPi-induced cytotoxicity, Applicants first examined whether downstream mediators of the FLT1 pathway are activated in PARPi-resistant tumors. Upon binding to VEGF family ligands (e.g., PGF), FLT1 activates growth and survival pathways, including AKT and STAT3 signaling, in immune and vascular smooth muscle cells (Bartoli et al, 2000; Bellik et al, 2005; Chen et al, 2008; Selvaraj et al, 2003; Tchaikovski et al, 2008). Therefore, PARPi-resistant Brca1-def and Bard1-def tumor cells expressing either control (Con) or Flt1-specific guide RNA (Flt1i) were exposed to recombinant PGF in vitro. Notably, Applicants observed FLT1-dependent activation of AKT, but not STAT3, upon PGF treatment (FIGS. 18A,B; FIGS. 19A,B). Likewise, AKT, but not STAT3, was also activated in PARPi-resistant tumors compared to PARPi-sensitive tumors in-vivo (FIGS. 18C,D; FIGS. 19C,D). Importantly, the combinations of either talazoparib-plus-Flt1i or talazoparib-plus-axitinib in-vivo significantly suppressed AKT activation in the PARPi-resistant tumors in mice (FIG. 18E-H). These results suggest that FLT1-AKT pathway activation allows breast tumor cells lacking BRCA1 function to escape PARPi-induced cytotoxicity in-vivo.

### The Cytotoxic Immune Response is Restored by the Combination of PARPi and FLT1 Blockade

Based on the known immune-modulatory functions of VEGF signaling (Zhang and Brekken, 2022) and the observation that PARPi-resistant tumors exhibit reduced numbers of CD8⁺ T cells (FIGS. 9A,B), Applicants asked whether inhibition of FLT1 signaling might indirectly impact the numbers of CD8⁺ T cells, and other immune cells, in PARPi-resistant tumors (FIG. 6). Indeed, increased CD8⁺ T-cell infiltration was observed in the PARPi-resistant tumors treated with talazoparib in combination with FLT1 blockade, either genetically (FIGS. 6A,B) or pharmacologically (FIGS. 6F,G). Importantly, the tumor regression observed in B6/129F1 mice upon combined treatment with talazoparib and FLT1 blockade (FIGS. 16C-E and FIG. 17I and FIG. 4 and FIGS. 11A,B) did not occur in T-cell-deficient nude-Foxn1^{nu} mice (FIGS. 6C-E and FIGS. 6H-J). Of note, CD4⁺ helper T cells, B220⁺ B cells and F4/80⁺ macrophages also showed a trend towards increased tumor infiltration upon FLT1 blockade in the Brca1-def and Bard1-def models (FIGS. 13A,B). These results demonstrate that re-sensitization of PARPi-resistant tumors by FLT1 blockade is a T-cell-dependent process, which may entail multiple changes in the immune status of the tumor microenvironment.

### Association Between FLT1 Activation in Human Tumor Cells at Pre-Treatment and Faster Progression on PARP Inhibitors in Breast Cancer Patients

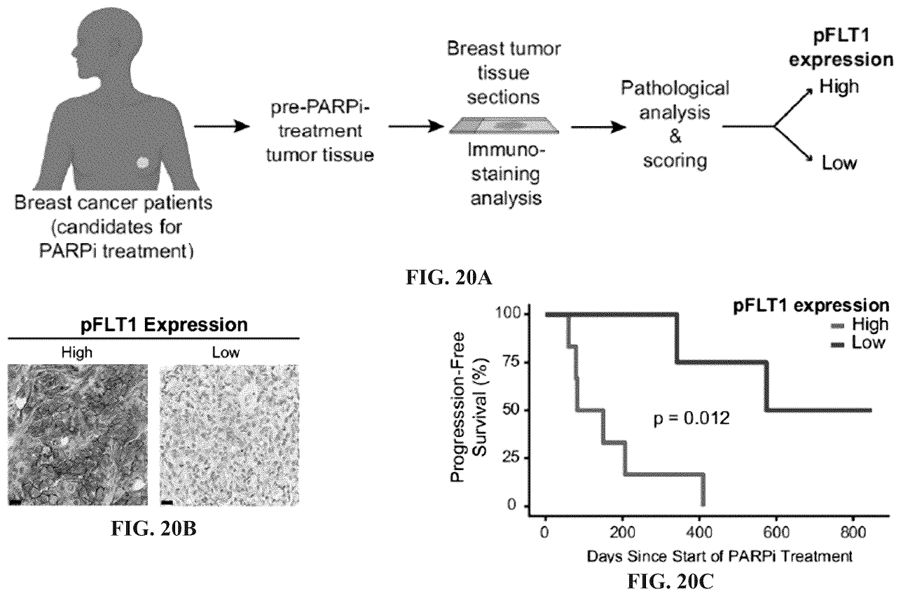

To validate the preclinical findings, Applicants performed pFLT1 and FLT1 immunostaining on tissue specimens that were obtained prior to PARPi treatment from 10 breast cancer patients harboring mutations in the BRCA1/2 or PALB2DNA damage response (DDR) genes (FIG. 20; FIGS. 14A,B; and Table 1). The immunostained samples were scored as either high or low expression for FLT1 and pFLT1 (activation) in tumor cells. Consistent with the preclinical observations (FIGS. 16 and 17), a blinded pathological examination revealed a statistically significant association between FLT1 expression (both activated and total) at pre-treatment and shorter progression-free survival (p=0.012 for pFLT1 and p=0.005 for total FLT1, FIG. 20A-C; FIGS. 14A,B) in patients with breast cancer. These findings suggest that FLT1 activation in human breast tumor cells (pre-treatment) is significantly associated with a higher risk of progression on PARPi, and thus pFLT1/FLT1 status in human breast tumors with BRCA1/2 or PALB2 mutations could serve as a biomarker to stratify patients for benefit from combination treatment with PARPi and FLT1 blockade.

### Discussion

The emergence of drug resistance is a major clinical challenge that limits the efficacy and durability of PARPi therapies for BRCA1/2-mutant breast cancer patients (Tung and Garber, 2022). Although PARPi treatment elicits 60% response rates and longer PFS compared to conventional chemotherapy agents (Litton et al, 2018; Robson et al, 2017), it fails to improve overall survival due to the onset of drug resistance (D'Andrea, 2018; Robson et al, 2017). Thus, overcoming drug resistance should improve the efficacy of PARPi treatment and extend the survival of breast cancer patients. Here, Applicants demonstrate that FLT1 signaling can potentially serve as a biomarker and therapeutic target to circumvent PARPi resistance in breast cancer patients.

In this study, Applicants describe new mouse models, based on Brca1-def and Bard1-def orthotopic allografts, that recapitulate the clinical response and progression phases of PARPi therapy observed in BRCA1/2-mutant breast cancer patients. In contrast to the well-established PARPi-resistance mechanisms based on restoration of BRCA1/2 pathway functions, Applicants identify an adaptive mechanism driven by PGF-FLT1-AKT signaling that protects Brca1- and Bard1-deficient breast tumor cells from PARPi-induced cell death in-vivo. Since PGF expression increases locally in the tumor milieu upon PARPi treatment (FIG. 2), it is possible that a minor subpopulation of Brca1- and Bard1-deficient tumor cells that express the PGF receptor FLT1 prior to PARPi treatment becomes enriched in the PARPi-resistant tumors. Therefore, the FLT1 pathway represents a vulnerability that can be targeted to overcome PARPi resistance. As such, the preclinical studies might offer a new biomarker-guided combination treatment option that includes a PARPi (e.g., talazoparib) and a VEGFR inhibitor (e.g., axitinib) to specifically target PARPi-resistant breast cancers that express FLT1.

The function of FLT1 varies by cell type and cellular context. Flt1 is normally expressed in endothelial cells, immune cells, and hematopoietic stem cells (HSCs) (Ferrara et al, 2003). In the context of angiogenesis, Flt1 is essential for the organization of embryonic vasculature but not for endothelial cell differentiation (Fong et al, 1995; Fong et al, 1999). FLT1 also functions as a signaling receptor in myeloid cells and HSCs by promoting their chemotaxis and migration in response to VEGF and/or PGF (Barleon et al, 1996; Clauss et al, 1996; Hattori et al, 2002; Hiratsuka et al, 1998). In the context of cancer, Flt1⁺ bone-marrow-derived hematopoietic progenitor cells promote the formation of pre-metastatic clusters and enhance tumor metastasis in mice (Kaplan et al, 2005). In addition, FLT1 signaling in macrophages activates an inflammatory response and promotes breast cancer metastasis (Qian et al, 2015). Here, Applicants show that FLT1 activation in tumor cells is also clinically relevant, in this case by promoting PARPi resistance in breast cancer through a combination of cell-intrinsic and -extrinsic pathways.

FLT1 expression has been previously reported in a subset of tumor cells where it promotes tumor growth by activating mitogenic pathways (Frank et al, 2011; Lesslie et al, 2006; Sopo et al, 2019; Wey et al, 2005; Wu et al, 2006; Yao et al, 2011). In line with these observations, the efficacy of anti-PGF antibodies strongly correlates with the expression of tumor-derived FLT1 but not with the inhibition of angiogenesis (Yao et al, 2011). This is consistent with the finding that FLT1 activation in the tumor cells represents a key in-vivo determinant of PARPi resistance in the breast cancer models. However, one point of distinction between the current study and the published literature is that Flt1-proficient and -deficient tumor cells show only modest growth differences in the absence of PARPi treatment in the tumor models. The difference in tumor growth only becomes pronounced after PARPi treatment, presumably when Flt1-deficient tumor cells are eliminated, and Flt1-proficient cells persist. Applicants, therefore, hypothesize that PARPi treatment leads to increased PGF expression in tumors, which in turn facilitates pro-survival signaling in FLT1-proficient (but not FLT1-deficient) tumor cells and culminates in the development of PARPi resistance.

In healthy, non-tumor-bearing mice, PARPi treatment reduces angiogenesis in response to growth factors (such as VEGF or PGF) in matrigel plug assays (Tentori et al, 2007). Interestingly, Applicants observed that PARPi causes increased numbers of CD31⁺ blood vessels and heightened VEGFR2 expression within PARPi-resistant tumors. In addition, VEGFR2 inhibition in combination with talazoparib only modestly reduced tumor growth in the Brca1-def and Bard1-def models, whereas Flt1 inhibition in combination with talazoparib resulted in prominent tumor regression. These data suggest that, in contrast to ovarian cancer models (Bizzaro et al, 2021), VEGFR2-induced angiogenesis does not appear to be a critical component of the growth and survival of PARPi-resistant breast cancer models.

The genetic experiments with Flt1 inhibition in cancer cells and the depletion experiments with VEGFR2 antibody allowed Applicants to differentiate between the contribution of FLT1 and VEGFR2 to PARPi resistance in breast cancer, the studies have revealed a novel function for FLT1 in promoting PARPi resistance. These findings are clinically relevant since the FLT1 pathway could serve as both biomarker and therapeutic target for overcoming PARPi resistance in breast cancer.

The data also suggest that the FLT1 pathway promotes PARPi resistance, at least in part, by suppressing a cytotoxic immune response in the tumor microenvironment. In particular, Applicants observed reduced numbers of CD8⁺ T cells in PARPi-resistant, but not PARPi-sensitive, tumors from the Brca1- and Bard1-deficient tumor models. Moreover, while FLT1 blockade with PARPi treatment is accompanied by increased CD8⁺ T-cell infiltration and tumor regression in immunocompetent mice, these effects were absent in T-cell-deficient nude-Foxn1^{nu} mice. The findings, therefore, suggest that FLT1 inhibition counters PARPi resistance through at least two distinct mechanisms. First, FLT1 blockade interrupts pro-survival PGF-FLT1-AKT signaling in tumor cells, and second, it increases T-cell infiltration and the immune response, possibly by altering the tumor-cell secretome and chemokine milieu. It is currently unclear how FLT1 inhibition in tumor cells alters the secretome and impacts T-cell numbers and function. Future studies will be needed to determine whether the suppression of T-cell cytotoxicity results from decreased chemotaxis, proliferation, or immunosuppression, or through interactions with other immunosuppressive cells in the tumor microenvironment. Although the PGF-FLT1 axis in tumor cells has not been investigated in the context of adaptive immunity, PGF secretion has been linked to immunosuppression (Albonici et al, 2019; Incio et al, 2016). For instance, PGF can induce dendritic-cell dysfunction and suppression of naïve CD4⁺ T-cell proliferation, thereby skewing T-cell responses toward Th2 (Lin et al, 2007). PGF can also immunosuppress CD8⁺ T cells by macrophage polarization (Albonici et al, 2019). It is possible that increased PGF expression after PARPi treatment reprograms immune cells to maintain an immunosuppressive tumor microenvironment, which can be further exacerbated by FLT1 signaling in tumor cells.

In addition to BRCA1/2-mutant breast cancer, PARPi is also approved for the treatment of ovarian, pancreatic, and prostate cancer. Interestingly, tumor-cell expression of FLT1 has been reported in ovarian, pancreatic, and prostate tumors (Boocock et al, 1995; Tsourlakis et al, 2015; Wey et al, 2005), suggesting that FLT1 signaling could potentially drive PARPi resistance in these cancers as well. PARPi agents have been approved to treat ovarian cancer patients with and without BRCA1/2 mutations and/or other HR deficiencies, and combined treatment with the PARPi olaparib and the VEGFA-selective blocker bevacizumab is FDA-approved for ovarian cancer as maintenance therapy (Le Saux et al, 2021; O'Malley et al, 2023). Importantly, the combination of olaparib and the pan-VEGFR inhibitor cediranib led to a significantly longer median progression-free survival (PFS, 16.5 vs. 8.2 months, hazard ratio of 0.50, p=0.007) compared to olaparib alone in a randomized Phase II study of relapsed high-grade ovarian cancer patients (Ivy et al, 2016; Liu et al, 2019). In a randomized 1:1:1 Phase III study in ovarian cancer patients comparing (1) cediranib-plus-olaparib, (2) olaparib, and (3) platinum chemotherapy (the standard of care), the treatment with cediranib-plus-olaparib showed similar clinical activity to platinum in the BRCA1/2-mutation-positive group (Liu et al, 2022). However, in the BRCA1/2-wild-type group, cediranib-plus-olaparib significantly prolonged PFS compared to olaparib alone in both trials, suggesting that alternative modes of action for this combination treatment exist beyond HR-dependent synthetic lethality. From clinical trial data, it will be interesting to retrospectively determine whether PARPi treatment activates PGF-FLT1 signaling in human ovarian tumor cells (similar to breast cancer models), which, when effectively blocked by cediranib-plus-olaparib, translates to clinical responses independent of BRCA1/2 status.

In the retrospective analyses of human breast tumors with BRCA1/2 or PALB2 mutations from patients, Applicants observed a strong association between FLT1 activation in human tumor cells at pre-treatment and subsequent development of PARPi resistance. Although the sample size is small, these findings lay the foundation for future biomarker studies to test whether FLT1 activation in pre-treatment biopsies can be used to stratify breast cancer patients at high risk for rapidly acquiring resistance to PARPi. Importantly, these patients might clinically benefit from the combined inhibition of PARP and FLT1. The study also highlights the need for testing non-genetic markers of PARPi resistance in patient samples by, for example, immunohistochemical staining for pFLT1 and FLT1 in tumor tissues pre- and post-treatment. Contrary to the notion that angiogenesis facilitates PARPi resistance (Alvarez Secord et al, 2021), the findings suggest that blocking angiogenic pathways driven by VEGFA or VEGFR2 alone might have only minimal impact on PARPi resistance. Instead, agents that disrupt the PGF/FLT1/AKT pathway, such as the pan-VEGFR inhibitor axitinib, are more likely to be effective at re-sensitizing PARPi-resistant breast cancers to PARPi. Since axitinib is already FDA-approved for metastatic renal-cell carcinoma (Tyler, 2012) and is currently being tested in combination with talazoparib in a Phase Ib/II clinical trial (NCT04337970) in renal cancer, the findings provide the rationale for repurposing this combination treatment in breast cancer patients with mutations in BRCA1/2 or PALB2. Moreover, if PGF-FLT1 signaling is increased in PARPi-resistant human breast cancers, future studies may validate the use of anti-PGF antibodies as another opportunity for therapeutic intervention to overcome PARPi resistance. This is particularly encouraging with renewed interest in TB-403, a monoclonal PGF-blocking antibody (Lassen et al, 2012; Martinsson-Niskanen et al, 2011) that showed promise in a Phase I clinical trial for medulloblastoma patients (Saulnier-Sholler et al, 2022). Collectively, the studies identify a previously unexplored role for FLT1 signaling in driving PARPi resistance in BRCA1/2-mutant breast cancers and suggest that pharmacological inactivation of FLT1 could greatly enhance the efficacy of PARPi treatment in patients.

### Methods

### Animal Studies

The treatment of mice in this study was conducted in compliance with the ethical regulations and guidelines set forth by the Columbia University Institutional Animal Care and Use Committee (IACUC), the U.S. National Research Council's Guide for the Care and Use of Laboratory Animals, and the US Public Health Service's Policy on Humane Care and Use of Laboratory Animals. The Institutional guidelines of Columbia University Medical Center (CUMC) Institute of Comparative Medicine were followed in these studies under approved protocol AABU4655. Mice were maintained in the CUMC barrier facility under conventional conditions with constant temperature and humidity and fed a standard diet (Labdiet 5053). The Brca1-def and Bard1-def cells derived from Brca1- or Bard1-conditionally deleted mice (Shakya et al, 2008) were authenticated, and the loss of Brca1 and Bard1 was confirmed by quantitative PCR analysis. These tumor cells were engineered to express luciferase for bioluminescence imaging and the hygromycin antibiotic resistance gene. Female B6129SF1/J mice (purchased from the Jackson Laboratory) and athymic nude-Foxn1^{nu} mice both aged 8-to-9 weeks (purchased from Envigo) were used in this study. These mice were injected with 5×10⁵ Brca1-def or Bard1-def tumor cells or their derivatives that were never exposed to talazoparib. Brca1-def and Bard1-def tumor cells (low passage number) were then injected into the mammary gland (orthotopic implantation), as previously described (Acharyya et al, 2012). Tumor growth was monitored weekly by using an electric caliper to measure the length and width of the tumors in millimeters. The tumor volume can be calculated using the formula (length×(width²))/2, where length and width represent the longest and shortest dimensions of the tumor, respectively. Mice were weighed weekly, monitored twice a week, and euthanized in accordance with the IACUC guidelines from Columbia University. The criteria for prompt euthanasia include weight loss of 20% or more, body-conditioning score (BCS) of 2 or less, signs of hunched posture from cachexia, impaired locomotion, or respiratory distress. Euthanasia was conducted by carbon dioxide inhalation with a secondary method of cervical dislocation. Timed collection of tumors was performed in matching cohorts and has been described in the relevant figure legends.

To generate Brca1-def and Bard1-def talazoparib-resistant tumor cells in-vivo, mice bearing Brca1-def and Bard1-def tumors were randomized and treated with PARPi. The PARPi talazoparib (Selleckchem) was solubilized in N,N-dimethylacetamide (Millipore), and then diluted in 6% Kolliphor® HS 15 (vehicle). Long-term talazoparib treatment via oral gavage was initiated at 0.3 mg/kg/day and administered five days a week. For the Brca1-def model, treatment started at 2 weeks post tumor-cell injection, and talazoparib-resistant tumors were collected 13 weeks after tumor-cell injection. For the Bard1-def model, treatment started at 1 week post tumor-cell injection, and talazoparib-resistant tumor cells were collected 5 weeks after injection. To isolate tumor cells, tumors were enzymatically dissociated using Dispase II (1 unit/mL, Roche) and collagenase Type I (2 mg/mL, Worthington). Non-tumor cells were eliminated by supplementing the culture medium with 200 μg/mL of hygromycin. For histological analysis, tumors were fixed in 4% paraformaldehyde in PBS for 24 h at 4° C., washed, and subsequently processed for histology.

For experiments involving antibody and drug treatments, 5×10⁵ talazoparib-resistant tumor cells (for both Brca1-def and Bard1-def models) were injected into the mammary gland of individual B6129SF1/J mice as described above. For in-vivo VEGFR2 (KDR) inhibition, the anti-VEGFR2 (BE0060, BioXCell) and the isotype control (BE0088, BioXCell) antibodies were diluted in a buffer (IP0070, BioXCell). Mice were randomly assigned to receive either 200 μg of VEGFR2 antibody or control antibody via intraperitoneal injection twice a week. Mice were euthanized at 4 weeks post tumor-cell injection for the Brca1-def model and at 3 weeks post tumor-cell injection for the Bard1-def model. Tumors were collected at the endpoint and processed for histology.

For genetic experiments involving Flt1 inhibition and Flt1 re-expression in talazoparib-resistant tumor cells in mice, talazoparib was administered as described above. Treatment started at 2 weeks (Brca1-def model) or 1 week (Bard1-def model) post tumor-cell injection. Mice were euthanized at their respective end points: at 4 weeks post tumor-cell injection for the Brca1-def model and at 3 weeks post tumor-cell injection for the Bard1-def model. Tumors were collected and subsequently fixed and processed as described above.

For VEGFR pharmacological inhibition studies, mice bearing talazoparib-resistant tumors from Brca1-def and Bard1-def models were randomized into multiple treatment groups. Talazoparib was solubilized as described previously. Axitinib (Selleckchem) was solubilized in 0.5% carboxymethylcellulose (w/v %). Drugs were administered in mice by oral gavage 5 days a week with a dose of 0.3 mg/kg/day of talazoparib and/or 30 mg/kg/day of axitinib. Treatments started at 2 weeks following tumor-cell injection for the Brca1-def model, and at 1 week following tumor-cell injection for the Bard1-def model. Brca1-def tumors were collected at 4 weeks post tumor-cell injection, and Bard1-def tumors were collected at 3 weeks post tumor-cell injection. Tumors were collected, subsequently fixed, and processed for histology.

### Immunostaining Analysis

Paraffin-embedded tumors from mice were sectioned at 5-μm thickness. Multiple sections from different depths of the tumors were used for immunostaining analysis. Slides were baked at 60° C. for 1 h and deparaffinized, rehydrated, and treated with 1% hydrogen peroxide for 10 min. Antigen retrieval was performed using either pH 6.0 citrate buffer (Vector Laboratories) or pH 9.0 Tris-based buffer (Vector Laboratories) in a steamer for 30 min, and endogenous avidin and biotin were blocked using avidin- and biotin-blocking reagents (Vector Laboratories), respectively. The slides were further blocked with BSA and goat or rabbit serum (depending on the species of the secondary antibodies), and tissue sections were incubated with primary antibodies, including antibodies against phospho-AKT (S473) (1:100, #4060, Cell Signaling Technology), KDR/VEGFR2 (1:2000, #9698, Cell Signaling Technology), VEGFA (1:300, #AF-493-NA, R&D Systems), PGF (1:300, AF465, R&D Systems), CD8a (1:200, #98941, Cell Signaling Technology), F4/80 (1:500, #70076, Cell Signaling Technology), B220 (1:400, #553085, BD Pharmingen), CD4 (1:200, #25229, Cell Signaling Technology), CD11C (1:250, #97585, Cell Signaling Technology), FOXP3 (1:100, #12653, Cell Signaling Technology), murine S100A9 (1:1000, #73425, Cell Signaling Technology), FLT4 (1:250, #AF743, R&D Systems), and phospho-Stat3 (S727) (1:100, #9134, Cell Signaling Technology), followed by incubation with the corresponding biotinylated secondary antibodies (1:250, Vector Laboratories). The ABC kit and DAB kit (Vector Laboratories) were used for detection following the manufacturer's instructions. Sections were subsequently counterstained with hematoxylin, dehydrated, and mounted using Cytoseal XYL (Richard-Allan Scientific) for microscopy and immunohistochemical analysis.

For automated immunostaining for CD31, pFLT1, and FLT1, paraffin-embedded tissue sections were sectioned at 5 μm and heated at 58° C. for 1 h. Samples were loaded into Leica Bond RX, and sections were dewaxed at 72° C. before being pretreated with EDTA-based epitope retrieval ER2 solution (Leica, AR9640) for 20 min at 100° C. The rabbit polyclonal antibodies against CD31 (0.08 μg/ml, Abcam, ab182981), pFLT1 (1:50, Millipore, 07-758), or FLT1 (2.5 μg/ml, Invitrogen, MA5-32045) were incubated for 60 min. Samples were then incubated with Leica Bond Post-Primary reagent (rabbit anti-mouse linker, included in the Polymer Refine Detection Kit (Leica, DS9800)) for 8 min, followed by incubation with Leica Bond Polymer (anti-rabbit HRP, included in Polymer Refine Detection Kit (Leica, DS9800)) for another 8 min. Mixed DAB reagent (Polymer Refine Detection Kit) was then incubated for 10 min, and hematoxylin (Refine Detection Kit) counterstaining was performed for 10 min. After staining, sample slides were washed in water, dehydrated using an ethanol gradient (70, 90, and 100%), washed three times in HistoClear II (National Diagnostics, HS-202), and mounted in Permount (Fisher Scientific, SP15). Immunostaining analysis for human samples was performed on sections of paraffin-embedded tissues, which included biopsies or resected samples. Staining was performed with antibodies against human pFLT1 (1:50, Millipore, 07-758) or FLT1 (2.5 μg/ml, Invitrogen, MA5-32045).

For calculating the staining intensity or the number of positively stained cells in tumor sections from mice, QuPath 0.3.2 software (qupath.github.io/) was used as previously described (Biswas et al, 2022). The image type was set as Brightfield (H-DAB) to count positive (pos.) cells, and the cell detection channel was set at Hematoxylin+DAB. The DAB threshold was adjusted and optimized for each antibody staining within the fast cell counts feature. To measure different staining intensities, tumor sections were selected and within the positive cell detection feature, detection image was set as optical density sum. Cell: DAB OD mean was used for scoring each compartment. Each threshold was adjusted on a batch-to-batch basis according to the staining condition to minimize false positive/negative readings. The data were normalized to the control group, thus setting the control to 1 in each case. The values for each of the experimental groups are compared relative to their respective control groups. The magnitude of the scale used in the figures is reflective of the relative expression of the experimental group over the control expression.

To assess the expression levels of pFLT1 and total FLT1 in tumor sections from patients, immunostainings were scored by a pathologist (HH) who was blinded to the sample details. The entire field of the tumor section was quantified for each sample. Staining that was scored as 0 or 1 was considered “low” and staining that was scored above 1 (and up to 3) was considered “high”, following the previous studies (Acharyya et al, 2012; Biswas et al, 2022).

### Cell Culture and In Vitro Assays

The Brca1-def and Bard1-def parental tumor cells and derivatives used in this study were cultured in DMEM media supplemented with 10% FBS and grown at 37° C. in a humidified CO₂ incubator (5% CO₂). All media were supplemented with 100 IU/mL penicillin and 100 μg/mL streptomycin (Life Technologies). They were authenticated by PCR analysis and tested for mycoplasma contamination.

The in vitro viability of sensitive vs. resistant Brca1-def and Bard1-def tumor cells with drug treatment was determined by cell viability assay using Promega CellTiter 96® AQueous One Solution Cell Proliferation Assay kit (Promega G3581) following the manufacturer's instructions. Briefly, 1000 Brca1-def sensitive or resistant tumor cells, or 500 Bard1-def sensitive or resistant tumor cells were plated into each well of a 96-well plate and allowed to grow in growth medium (DMEM supplemented with 10% FBS and Pen-Strep) overnight at 37° C. in a humidified, 5% CO₂ incubator (Keung et al, 2020). Subsequently, cells were treated with either DMSO (vehicle) or various concentrations of talazoparib (prepared in growth medium) ranging from 0 to 10,000 nM for 7 days, replacing with fresh drug-containing media after every 3 days. After 7 days, the drug-containing medium was replaced by adding 100 μl of phenol red-free growth medium and 20 μl of CellTiter 96 AQueous One Solution Reagent (Promega) to each well. After incubation for 1-2 h at 37° C. in a humidified, 5% CO₂ incubator, the absorbance of colored-formazan formation (by metabolically active cells) was measured at 490 nm using a plate reader. The viability was calculated as a percent of absorbance (viable cells) in vehicle-treated controls (designated as 100% viability).

For treatment of cells with recombinant PGF, talazoparib-resistant Brca1-def and Bard1-def Flt1-proficient and their Flt1-deficient tumor cells were plated and cultured overnight in growth medium (DMEM medium supplemented with 10% FBS). For the Brca1-def group, cells were then washed three times with HBSS buffer, and serum-free media was added to the plate for 4 h. Cells were subsequently washed once with HBSS and then treated with 50 ng/ml of mouse recombinant PGF (R&D Systems, #465-PL-010) in serum-free medium for 15 s. Cells were then collected, lysed, and prepared for immunoblot analysis. For Bard1-def talazoparib-resistant and its Flt1i-deficient derivatives, cells were plated and, after an overnight culturing in 10% FBS DMEM medium, washed three times with HBSS buffer and replenished with serum-free medium for 24 h. Cells were then treated with 50 ng/ml mouse recombinant PGF in a serum-free medium for 10 min. Cells were thereafter harvested, and lysates were prepared for immunoblot analysis described below.

### Immunoblot Analysis

Cells were washed with cold PBS, collected in lysis buffer consisting of 25 mM Tris, pH 7.5, 150 mM NaCl, 1% Triton X-100 (v/v), 0.5% SDS (w/v), and supplemented with protease inhibitor cocktail (Roche) and phosphatase inhibitor cocktail (Thermo Scientific). Subsequently, the cell suspension was sonicated, and supernatant fraction of cell lysates was collected by centrifugation at 18,000×g for 10 min at 4° C. Protein concentration in the supernatant was determined by the BCA protein assay kit (Pierce), and protein sample was prepared by mixing with Laemmli SDS-PAGE reducing sample buffer and incubated at 98° C. for 5 min. After cooling down to room temperature, an equal amount of total protein from each sample was resolved on 4-20% precast polyacrylamide gel (Bio-Rad Cat. #4561093) by electrophoresis. Protein bands were transferred to nitrocellulose membranes and blocked with 5% milk in TBST (Tris-buffered saline containing 0.1% Tween-20) by incubating for 1 h at room temperature with constant agitation. Blots were then incubated overnight with primary antibodies (diluted using 2.5% milk in TBST) against pAKT (S473) (1:1000, Cell Signaling Technology, #4060), pSTAT3 (Ser 727) (1:1000, Cell Signaling Technology, #9134) generated in rabbit, or with a mouse mAb against β-actin (1:2000, Sigma, A1978). The membranes were washed three times with TBST, 5 min each time, and incubated for 1 h at room temperature with the corresponding secondary antibodies conjugated with horseradish peroxidase (HRP). The membranes were then washed three times with TBST for 5 min each time before they were developed using an enhanced chemiluminescence (ECL) substrate (Bio-Rad), and specific protein bands were visualized on a Bio-Rad ChemiDoc Imaging System (Bio-Rad). Blotted membranes were stripped with stripping buffer (Thermo Scientific, #46430) for 15 min and subsequently blocked for 1 h with 5% milk in TBST. Membranes were then incubated overnight at 4° C. with antibodies against total AKT (1:1000, Cell Signaling Technology, #4691) and total STAT3 (1:1000, Cell Signaling Technology, #4904), followed by the corresponding horseradish peroxidase (HRP)-conjugated secondary antibodies. The membranes were developed using an ECL substrate (Bio-Rad), and protein bands were visualized on a Bio-Rad ChemiDoc Imaging System (Bio-Rad).

RNA Isolation and qRT-PCR

Total RNA was isolated using TRIzol and RNeasy Mini Kit as previously described (Biswas et al, 2022). RNA (500 ng) was then reverse-transcribed to cDNA using a cDNA Synthesis Kit (Applied Biosystems; Thermo Fisher Scientific). qRT-PCR was performed with 10 ng of cDNA per sample using gene-specific primers and SYBR Green PCR master mix (Applied Biosystems; Thermo Fisher Scientific). GAPDH primers were used as an internal control. qPCR was run using Applied Biosystems 7500 Real-Time PCR system (Applied Biosystems; Thermo Fisher Scientific), and data were exported to Excel (Microsoft) for gene expression analysis using the 2^{-ΔΔCt} method. The qRT-PCR primer sequences used in this study are shown below:

mFlt1
forward primer:
(SEQ ID NO. 1)
5-TGGCTCTACGACCTTAGACTG-3
reverse primer:
(SEQ ID NO. 2)
5-CAGGTTTGACTTGTCTGAGGTT-3
mGapdh
forward primer:
(SEQ ID NO. 3)
5-AGGTCGGTGTGAACGGATTTG-3
reverse primer:
(SEQ ID NO. 4)
5-TGTAGACCATGTAGTTGAGGTCA-3

### Gene Repression by CRISPR

The expression of Flt1 was suppressed by CRISPR/dCas9-KRAB-mediated gene editing following a previously described method (Biswas et al, 2022). Applicants designed the gRNA1 sequence (5′-CAGCGCGTAAGGCAAGACCG-3′, SEQ ID NO. 5) and gRNA2 sequence (5′-CACCACTAGCACTACCTCCC-3′, SEQ ID NO. 6) using the CRISPR-ERA online tool (crispr-era.stanford.edu). The forward and reverse oligos were designed based on the gRNA sequence and were then annealed and cloned into the BsmBI-digested LentiCRISPRv2-SFFV-KRAB-dCas9 (Biswas et al, 2022) following the procedure outlined by Feng Zhang's group (Sanjana et al, 2014). Applicants confirmed the positive clones by PCR using the human U6 forward primer and the reverse oligo of the corresponding gRNA sequence. Applicants produced lentivirus by transfecting the gRNA-cloned lentiviral vector into the Lenti-X 293 T cells line (Takara, cat #632180) using the third-generation packaging system. Target cells were transduced with viral supernatant (after passing through a 0.45-micron syringe filter) and selected at 48 h post-transduction with puromycin at a final concentration of 8 μg/ml. The efficiency of knockdown was tested by RT-PCR using mouse-Flt1-specific primers.

### Expression of Flt1

To re-express the Flt1 gene in Flt1-suppressed (CRISPRi) cells, Applicants cloned the cDNA encoding mouse-Flt1 into the lentiviral plasmid pLV-EF1a-IRES-Blast (Addgene #85133; ref. Hayer et al) within BamHI/EcoRI sites. Subsequently, Applicants produced lentiviral particles with the construct using a third-generation packaging system, following standard procedure. To stably express Flt1 in Flt1-repressed cells, Applicants transduced Flt1-repressed cells with lentiviral particles carrying Flt1 cDNA and selected with blasticidine S hydrochloride antibiotic after 48 h of viral transduction. After 1 week of antibiotic selection, Applicants confirmed the expression of Flt1 at RNA and protein levels by real-time PCR and western blotting, respectively.

### Patient Samples

Human tumor tissue samples from 10 breast cancer patients were collected prior to PARP inhibitor treatment either at the Memorial Sloan Kettering Cancer Center or Emory University School of Medicine in accordance with approved protocols from their respective institutional review boards (IRB), ensuring the protection of patient privacy and conformed to the principles set out in the WMA Declaration of Helsinki and the Department of Health and Human Services Belmont Report. Informed consent was obtained from all subjects participating in this study. The research was conducted following ethical regulations specified by the IRB, and de-identified information was provided to the research team. Tissue sections were used for immunostaining analysis. Appendix Table S1 provides de-identified information about the mutations of tumors from breast cancer patients and indicates whether the tumors had high or low levels of pFLT1. Sections were stained with antibodies against either pFLT1 or total FLT1 and scored as described in the Immunostaining Analysis section after pathological review.

### Statistical Analysis

Statistical significance was determined using Prism 9 software (GraphPad Software) to perform the following analyses: (1) unpaired, two-tailed Student's t-test, (2) unpaired Welch's t-test, (3) unpaired Mann-Whitney t-test, (4) one-way ANOVA with post-hoc Tukey's test, and (5) log-rank test. All values were determined as the mean±SEM, and P values<0.05 were considered statistically significant. Experimental results were obtained after repeating them three times for reproducibility and rigor. Power analysis was performed for sample size calculation for animal experiments. The Kaplan-Meier method was utilized to generate progression-free survival (PFS) curves. To assess the null hypothesis suggesting no difference between the two curves, the log-rank test was employed through the survdiff( ) function in R.

### EXAMPLE 2 REFERENCES

Acharyya S, Oskarsson T, Vanharanta S, Malladi S, Kim J, Morris P G, Manova-Todorova K, Leversha M, Hogg N, Seshan V E et al (2012) A CXCL1 paracrine network links cancer chemoresistance and metastasis. Cell 150:165-178 10.1016/j.cell.2012.04.042.
Albonici L, Giganti M G, Modesti A, Manzari V, Bei R (2019) Multifaceted role of the placental growth factor (PIGF) in the antitumor immune response and cancer progression. Int J Mol Sci 20:2970 10.3390/ijms20122970.
Alvarez Secord A, O'Malley D M, Sood A K, Westin S N, Liu J F (2021) Rationale for combination PARP inhibitor and antiangiogenic treatment in advanced epithelial ovarian cancer: a review. Gynecol Oncol 162:482-495 10.1016/j.ygyno.2021.05.018.
Barber L J, Sandhu S, Chen L, Campbell J, Kozarewa I, Fenwick K, Assiotis I, Rodrigues D N, Reis Filho J S, Moreno V et al (2013) Secondary mutations in BRCA2 associated with clinical resistance to a PARP inhibitor. J Pathol 229:422-429 10.1002/path.4140.
Barleon B, Sozzani S, Zhou D, Weich H A, Mantovani A, Marme D (1996) Migration of human monocytes in response to vascular endothelial growth factor (VEGF) is mediated via the VEGF receptor flt-1. Blood 87:3336-3343 10.1182/blood.V87.8.3336.bloodjournal8783336.
Bartoli M, Gu X, Tsai N T, Venema R C, Brooks S E, Marrero M B, Caldwell R B (2000) Vascular endothelial growth factor activates STAT proteins in aortic endothelial cells. J Biol Chem 275:33189-33192 10.1074/jbc.C000318200.
Bellik L, Vinci M C, Filippi S, Ledda F, Parenti A (2005) Intracellular pathways triggered by the selective FLT-1-agonist placental growth factor in vascular smooth muscle cells exposed to hypoxia. Br J Pharmacol 146:568-575 10.1038/sj.bjp.0706347.
Berti M, Cortez D, Lopes M (2020) The plasticity of DNA replication forks in response to clinically relevant genotoxic stress. Nat Rev Mol Cell Biol 21:633-651 10.1038/s41580-020-0257-5.
Bhin J, Paes Dias M, Gogola E, Rolfs F, Piersma S R, de Bruijn R, de Ruiter J R, van den Broek B, Duarte A A, Sol W et al (2023) Multi-omics analysis reveals distinct non-reversion mechanisms of PARPi resistance in BRCA1-versus BRCA2-deficient mammary tumors. Cell Rep 42:112538 10.1016/j.celrep.2023.112538.
Biswas A K, Han S, Tai Y, Ma W, Coker C, Quinn S A, Shakri A R, Zhong T J, Scholze H, Lagos G G et al (2022) Targeting S100A9-ALDH1A1-retinoic acid signaling to suppress brain relapse in EGFR-mutant lung cancer. Cancer Discov 12:1002-1021 10.1158/2159-8290.CD-21-0910.
Bizzaro F, Fuso Nerini I, Taylor M A, Anastasia A, Russo M, Damia G, Guffanti F, Guana F, Ostano P, Minoli L et al (2021) VEGF pathway inhibition potentiates PARP inhibitor efficacy in ovarian cancer independent of BRCA status. J Hematol Oncol 14:186 10.1186/s13045-021-01196-x.
Boocock C A, Charnock-Jones D S, Sharkey A M, McLaren J, Barker P J, Wright K A, Twentyman P R, Smith S K (1995) Expression of vascular endothelial growth factor and its receptors flt and KDR in ovarian carcinoma. J Natl Cancer Inst 87:506-516 10.1093/jnci/87.7.506.
Brose M S, Rebbeck T R, Calzone K A, Stopfer J E, Nathanson K L, Weber B L (2002) Cancer risk estimates for BRCA1 mutation carriers identified in a risk evaluation program. J Natl Cancer Inst 94:1365-1372 10.1093/jnci/94.18.1365
Bryant H E, Schultz N, Thomas H D, Parker K M, Flower D, Lopez E, Kyle S, Meuth M, Curtin N J, Helleday T (2005) Specific killing of BRCA2-deficient tumours with inhibitors of poly(ADP-ribose) polymerase. Nature 434:913-917 10.1038/nature03443.
Chen S H, Murphy D A, Lassoued W, Thurston G, Feldman M D, Lee W M (2008) Activated STAT3 is a mediator and biomarker of VEGF endothelial activation. Cancer Biol Ther 7:1994-2003 10.4161/cbt.7.12.6967.
Clauss M, Weich H, Breier G, Knies U, Rockl W, Waltenberger J, Risau W (1996) The vascular endothelial growth factor receptor Flt-1 mediates biological activities. Implications for a functional role of placenta growth factor in monocyte activation and chemotaxis. J Biol Chem 271:17629-17634 10.1074/jbc.271.30.17629.
Comen E, Davids M, Kirchhoff T, Hudis C, Offit K, Robson M (2011) Relative contributions of BRCA1 and BRCA2 mutations to “triple-negative” breast cancer in Ashkenazi Women. Breast Cancer Res Treat 129:185-190 10.1007/s10549-011-1433-2.
Comen E A, Robson M (2010) Inhibition of poly (ADP)-ribose polymerase as a therapeutic strategy for breast cancer. Oncology 24:55-62
Cruz C, Castroviejo-Bermejo M, Gutierrez-Enriquez S, Llop-Guevara A, Ibrahim Y H, Gris-Oliver A, Bonache S, Morancho B, Bruna A, Rueda O M et al (2018) RAD51 foci as a functional biomarker of homologous recombination repair and PARP inhibitor resistance in germline BRCA-mutated breast cancer. Ann Oncol 29:1203-1210 10.1093/annonc/mdy099.
D'Andrea A D (2018) Mechanisms of PARP inhibitor sensitivity and resistance. DNA Repair 71:172-176 10.1016/j.dnarep.2018.08.021.
Dias M P, Moser S C, Ganesan S, Jonkers J (2021) Understanding and overcoming resistance to PARP inhibitors in cancer therapy. Nat Rev Clin Oncol 18:773-791 10.1038/s41571-021-00532-x.
Ding L, Kim H J, Wang Q, Kearns M, Jiang T, Ohlson C E, Li B B, Xie S, Liu J F, Stover E H et al (2018) PARP inhibition elicits STING-dependent antitumor immunity in Brca1-deficient ovarian cancer. Cell Rep 25:2972-2980.e2975 10.1016/j.celrep.2018.11.054.
Drost R, Dhillon K K, van der Gulden H, van der Heijden I, Brandsma I, Cruz C, Chondronasiou D, Castroviejo-Bermejo M, Boon U, Schut E et al (2016) BRCA1185delAG tumors may acquire therapy resistance through expression of RING-less BRCA1. J Clin Invest 126:2903-2918 10.1172/JCI70196.
Farmer H, McCabe N, Lord C J, Tutt A N, Johnson D A, Richardson T B, Santarosa M, Dillon K J, Hickson I, Knights C et al (2005) Targeting the DNA repair defect in BRCA mutant cells as a therapeutic strategy. Nature 434:917-921 10.1038/nature03445.
Ferrara N, Gerber H P, LeCouter J (2003) The biology of VEGF and its receptors. Nat Med 9:669-676 10.1038/nm0603-669.
Fong G H, Rossant J, Gertsenstein M, Breitman M L (1995) Role of the Flt-1 receptor tyrosine kinase in regulating the assembly of vascular endothelium. Nature 376:66-70 10.1038/376066a0.
Fong G H, Zhang L, Bryce D M, Peng J (1999) Increased hemangioblast commitment, not vascular disorganization, is the primary defect in flt-1 knock-out mice. Development 126:3015-3025 10.1242/dev.126.13.3015.
Ford D, Easton D F, Bishop D T, Narod S A, Goldgar D E (1994) Risks of cancer in BRCA1-mutation carriers. Breast Cancer Linkage Consortium. Lancet 343:692-695 10.1016/S0140-6736 (94) 91578-4.
Ford D, Easton D F, Stratton M, Narod S, Goldgar D, Devilee P, Bishop D T, Weber B, Lenoir G, Chang-Claude J et al (1998) Genetic heterogeneity and penetrance analysis of the BRCA1 and BRCA2 genes in breast cancer families. The Breast Cancer Linkage Consortium. Am J Hum Genet 62:676-689 10.1086/301749.
Foulkes W D, Metcalfe K, Sun P, Hanna W M, Lynch H T, Ghadirian P, Tung N, Olopade O I, Weber B L, McLennan J et al (2004) Estrogen receptor status in BRCA1- and BRCA2-related breast cancer: the influence of age, grade, and histological type. Clin Cancer Res 10:2029-2034 10.1158/1078-0432.CCR-03-1061.
Frank N Y, Schatton T, Kim S, Zhan Q, Wilson B J, Ma J, Saab K R, Osherov V, Widlund H R, Gasser M et al (2011) VEGFR-1 expressed by malignant melanoma-initiating cells is required for tumor growth. Cancer Res 71:1474-1485 10.1158/0008-5472.CAN-10-1660.
Galindo-Campos M A, Bedora-Faure M, Farres J, Lescale C, Moreno-Lama L, Martinez C, Martin-Caballero J, Ampurdanes C, Aparicio P, Dantzer F et al (2019) Coordinated signals from the DNA repair enzymes PARP-1 and PARP-2 promotes B-cell development and function. Cell Death Differ 26:2667-2681 10.1038/s41418-019-0326-5.
Groelly F J, Fawkes M, Dagg R A, Blackford A N, Tarsounas M (2023) Targeting DNA damage response pathways in cancer. Nat Rev Cancer 23:78-94 10.1038/s41568-022-00535-5.
Hall J M, Lee M K, Newman B, Morrow J E, Anderson L A, Huey B, King M C (1990) Linkage of early-onset familial breast cancer to chromosome 17q21. Science 250:1684-1689 10.1126/science.2270482.
Hattori K, Heissig B, Wu Y, Dias S, Tejada R, Ferris B, Hicklin D J, Zhu Z, Bohlen P, Witte L et al (2002) Placental growth factor reconstitutes hematopoiesis by recruiting VEGFR1 (+) stem cells from bone-marrow microenvironment. Nat Med 8:841-849 10.1038/nm740.
Henneman L, van Miltenburg M H, Michalak E M, Braumuller T M, Jaspers J E, Drenth A P, de Korte-Grimmerink R, Gogola E, Szuhai K, Schlicker A et al (2015) Selective resistance to the PARP inhibitor olaparib in a mouse model for BRCA1-deficient metaplastic breast cancer. Proc Natl Acad Sci USA 112:8409-8414 10.1073/pnas.1500223112.
Hiratsuka S, Minowa O, Kuno J, Noda T, Shibuya M (1998) Flt-1 lacking the tyrosine kinase domain is sufficient for normal development and angiogenesis in mice. Proc Natl Acad Sci USA 95:9349-9354 10.1073/pnas.95.16.9349.
Hobbs E A, Litton J K, Yap T A (2021) Development of the PARP inhibitor talazoparib for the treatment of advanced BRCA1 and BRCA2 mutated breast cancer. Expert Opin Pharmacother 22:1825-1837 10.1080/14656566.2021.1952181.
Incio J, Tam J, Rahbari N N, Suboj P, McManus D T, Chin S M, Vardam T D, Batista A, Babykutty S, Jung K et al (2016) PIGF/VEGFR-1 signaling promotes macrophage polarization and accelerated tumor progression in obesity. Clin Cancer Res 22:2993-3004 10.1158/1078-0432.CCR-15-1839.
Ivy S P, Liu J F, Lee J M, Matulonis U A, Kohn E C (2016) Cediranib, a pan-VEGFR inhibitor, and olaparib, a PARP inhibitor, in combination therapy for high grade serous ovarian cancer. Expert Opin Investig Drugs 25:597-611 10.1517/13543784.2016.1156857.
Kaplan R N, Riba R D, Zacharoulis S, Bramley A H, Vincent L, Costa C, MacDonald D D, Jin D K, Shido K, Kerns S A et al (2005) VEGFR1-positive haematopoietic bone marrow progenitors initiate the pre-metastatic niche. Nature 438:820-827 10.1038/nature04186.
Keung M Y, Wu Y, Badar F, Vadgama J V (2020) Response of breast cancer cells to PARP inhibitors is independent of BRCA status. J Clin Med 9:940 10.3390/jcm9040940.
Lassen U, Nielsen D L, Sorensen M, Winstedt L, Niskanen T, Stenberg Y, Pakola S, Stassen J M, Glazer S (2012) A phase I, dose-escalation study of TB-403, a monoclonal antibody directed against PIGF, in patients with advanced solid tumours. Br J Cancer 106:678-684 10.1038/bjc.2011.609.
Le Saux O, Vanacker H, Guermazi F, Carbonnaux M, Romeo C, Larrouquere L, Tredan O, Ray-Coquard I (2021) Poly(ADP-ribose) polymerase inhibitors in combination with anti-angiogenic agents for the treatment of advanced ovarian cancer. Future Oncol 17:2291-2304 10.2217/fon-2021-0059.
Lesslie D P, Summy J M, Parikh N U, Fan F, Trevino J G, Sawyer T K, Metcalf C A, Shakespeare W C, Hicklin D J, Ellis L M et al (2006) Vascular endothelial growth factor receptor-1 mediates migration of human colorectal carcinoma cells by activation of Src family kinases. Br J Cancer 94:1710-1717 10.1038/sj.bjc.6603143.
Lim B W X, Li N, Mahale S, McInerny S, Zethoven M, Rowley S M, Huynh J, Wang T, Lee J E A, Friedman M et al (2023) Somatic inactivation of breast cancer predisposition genes in tumors associated with pathogenic germline variants. J Natl Cancer Inst 115:181-189 10.1093/jnci/djac196.
Lin K K, Harrell M I, Oza A M, Oaknin A, Ray-Coquard I, Tinker A V, Helman E, Radke M R, Say C, Vo L T et al (2019) BRCA reversion mutations in circulating tumor DNA predict primary and acquired resistance to the PARP inhibitor rucaparib in high-grade ovarian carcinoma. Cancer Discov 9:210-219 10.1158/2159-8290.CD-18-0715.
Lin Y L, Liang Y C, Chiang B L (2007) Placental growth factor down-regulates type 1 T helper immune response by modulating the function of dendritic cells. J Leukoc Biol 82:1473-1480 10.1189/jlb.0307164.
Litton J K, Rugo H S, Ettl J, Hurvitz S A, Goncalves A, Lee K H, Fehrenbacher L, Yerushalmi R, Mina L A, Martin M et al (2018) Talazoparib in patients with advanced breast cancer and a germline BRCA mutation. N Engl J Med 379:753-763 10.1056/NEJMoa1802905.
Liu J F, Barry W T, Birrer M, Lee J M, Buckanovich R J, Fleming G F, Rimel B J, Buss M K, Nattam S R, Hurteau J et al (2019) Overall survival and updated progression-free survival outcomes in a randomized phase I I study of combination cediranib and olaparib versus olaparib in relapsed platinum-sensitive ovarian cancer. Ann Oncol 30:551-557 10.1093/annonc/mdz018.
Liu J F, Brady M F, Matulonis U A, Miller A, Kohn E C, Swisher E M, Cella D, Tew W P, Cloven N G, Muller C Y et al (2022) Olaparib with or without cediranib versus platinum-based chemotherapy in recurrent platinum-sensitive ovarian cancer (NRG-GY004): a randomized, open-label, phase III trial. J Clin Oncol 40:2138-2147 10.1200/JCO.21.02011.
Martinsson-Niskanen T, Riisbro R, Larsson L, Winstedt L, Stenberg Y, Pakola S, Stassen J M, Glazer S (2011) Monoclonal antibody TB-403: a first-in-human, Phase I, double-blind, dose escalation study directed against placental growth factor in healthy male subjects. Clin Ther 33:1142-1149 10.1016/j.clinthera.2011.08.007.
McCabe N, Turner N C, Lord C J, Kluzek K, Bialkowska A, Swift S, Giavara S, O'Connor M J, Tutt A N, Zdzienicka M Z et al (2006) Deficiency in the repair of DNA damage by homologous recombination and sensitivity to poly(ADP-ribose) polymerase inhibition. Cancer Res 66:8109-8115 10.1158/0008-5472.CAN-06-0140.
Miki Y, Swensen J, Shattuck-Eidens D, Futreal P A, Harshman K, Tavtigian S, Liu Q, Cochran C, Bennett L M, Ding W et al (1994) A strong candidate for the breast and ovarian cancer susceptibility gene BRCA1. Science 266:66-71 10.1126/science.7545954.
Moreno-Lama L, Galindo-Campos M A, Martinez C, Comerma L, Vazquez I, Vernet-Tomas M, Ampurdanes C, Lutfi N, Martin-Caballero J, Dantzer F et al (2020) Coordinated signals from PARP-1 and PARP-2 are required to establish a proper T cell immune response to breast tumors in mice. Oncogene 39:2835-2843 10.1038/s41388-020-1175-x.
Nik-Zainal S, Davies H, Staaf J, Ramakrishna M, Glodzik D, Zou X, Martincorena I, Alexandrov L B, Martin S, Wedge D C et al (2016) Landscape of somatic mutations in 560 breast cancer whole-genome sequences. Nature 534:47-54 10.1038/nature17676.
O'Malley D M, Krivak T C, Kabil N, Munley J, Moore K N (2023) PARP inhibitors in ovarian cancer: a review. Target Oncol 18:471-503 10.1007/s11523-023-00970-w.
Pettitt S J, Frankum J R, Punta M, Lise S, Alexander J, Chen Y, Yap T A, Haider S, Tutt A N J, Lord C J (2020) Clinical BRCA1/2 reversion analysis identifies hotspot mutations and predicted neoantigens associated with therapy resistance. Cancer Discov 10:1475-1488 10.1158/2159-8290.CD-19-1485.
Powell S N (2016) BRCA1 loses the ring but lords over resistance. J Clin Invest 126:2802-2804 10.1172/JCI89209.
Qian B Z, Zhang H, Li J, He T, Yeo E J, Soong D Y, Carragher N O, Munro A, Chang A, Bresnick A R et al (2015) FLT1 signaling in metastasis-associated macrophages activates an inflammatory signature that promotes breast cancer metastasis. J Exp Med 212:1433-1448 10.1084/jem.20141555.
Robson M, Im S A, Senkus E, Xu B, Domchek S M, Masuda N, Delaloge S, Li W, Tung N, Armstrong A et al (2017) Olaparib for metastatic breast cancer in patients with a germline BRCA mutation. N Engl J Med 377:523-533 10.1056/NEJMoa1706450.
Rottenberg S, Jaspers J E, Kersbergen A, van der Burg E, Nygren A O, Zander S A, Derksen P W, de Bruin M, Zevenhoven J, Lau A et al (2008) High sensitivity of BRCA1-deficient mammary tumors to the PARP inhibitor AZD2281 alone and in combination with platinum drugs. Proc Natl Acad Sci USA 105:17079-17084 10.1073/pnas.0806092105.
Sanjana N E, Shalem O, Zhang F (2014) Improved vectors and genome-wide libraries for CRISPR screening. Nat Methods 11:783-784 10.1038/nmeth.3047.
Saulnier-Sholler G, Duda D G, Bergendahl G, Ebb D, Snuderl M, Laetsch T W, Michlitsch J, Hanson D, Isakoff M S, Bielamowicz K et al (2022) A phase I trial of TB-403 in relapsed medulloblastoma, neuroblastoma, ewing sarcoma, and alveolar rhabdomyosarcoma. Clin Cancer Res 28:3950-3957 10.1158/1078-0432.CCR-22-1169.
Scully R, Livingston D M (2000) In search of the tumour-suppressor functions of BRCA1 and BRCA2. Nature 408:429-432 10.1038/35044000.
Selvaraj S K, Giri R K, Perelman N, Johnson C, Malik P, Kalra V K (2003) Mechanism of monocyte activation and expression of proinflammatory cytochemokines by placenta growth factor. Blood 102:1515-1524 10.1182/blood-2002-11-3423.
Shakri A R, James Zhong T, Ma W, Coker C, Hegde R, Scholze H, Chin V, Szabolcs M, Hibshoosh H, Tanji K et al (2020) Aberrant Zip14 expression in muscle is associated with cachexia in a Bard1-deficient mouse model of breast cancer metastasis. Cancer Med 9:6766-6775 10.1002/cam4.3242.
Shakya R, Szabolcs M, McCarthy E, Ospina E, Basso K, Nandula S, Murty V, Baer R, Ludwig T (2008) The basal-like mammary carcinomas induced by Brca1 or Bard1 inactivation implicate the BRCA1/BARD1 heterodimer in tumor suppression. Proc Natl Acad Sci USA 105:7040-7045 10.1073/pnas.0711032105.
Shen J, Zhao W, Ju Z, Wang L, Peng Y, Labrie M, Yap T A, Mills G B, Peng G (2019) PARPi triggers the STING-dependent immune response and enhances the therapeutic efficacy of immune checkpoint blockade independent of BRCAness. Cancer Res 79:311-319 10.1158/0008-5472.CAN-18-1003.
Shibuya M (2001) Structure and function of VEGF/VEGF-receptor system involved in angiogenesis. Cell Struct Funct 26:25-35 10.1247/csf.26.25.
Sopo M, Anttila M, Hamalainen K, Kivela A, Yla-Herttuala S, Kosma V M, Keski-Nisula L, Sallinen H (2019) Expression profiles of VEGF-A, VEGF-D and VEGFR1 are higher in distant metastases than in matched primary high grade epithelial ovarian cancer. BMC Cancer 19:584 10.1186/s12885-019-5757-3.
Tchaikovski V, Fellbrich G, Waltenberger J (2008) The molecular basis of VEGFR-1 signal transduction pathways in primary human monocytes. Arterioscler Thromb Vasc Biol 28:322-328 10.1161/ATVBAHA.107.158022.
Tentori L, Lacal P M, Muzi A, Dorio A S, Leonetti C, Scarsella M, Ruffini F, Xu W, Min W, Stoppacciaro A et al (2007) Poly(ADP-ribose) polymerase (PARP) inhibition or PARP-1 gene deletion reduces angiogenesis. Eur J Cancer 43:2124-2133 10.1016/j.ejca.2007.07.010.
Tobalina L, Armenia J, Irving E, O'Connor M J, Forment J V (2021) A meta-analysis of reversion mutations in BRCA genes identifies signatures of DNA end-joining repair mechanisms driving therapy resistance. Ann Oncol 32:103-112 10.1016/j.annonc.2020.10.470.
Tsourlakis M C, Khosrawi P, Weigand P, Kluth M, Hube-Magg C, Minner S, Koop C, Graefen M, Heinzer H, Wittmer C et al (2015) VEGFR-1 overexpression identifies a small subgroup of aggressive prostate cancers in patients treated by prostatectomy. Int J Mol Sci 16:8591-8606 10.3390/ijms16048591
Tung N, Garber J E (2022) PARP inhibition in breast cancer: progress made and future hopes. NPJ Breast Cancer 8:47 10.1038/s41523-022-00411-3.
Tyler T (2012) Axitinib: newly approved for renal cell carcinoma. J Adv Pract Oncol 3:333-335
van der Kolk D M, de Bock G H, Leegte B K, Schaapveld M, Mourits M J, de Vries J, van der Hout A H, Oosterwijk J C (2010) Penetrance of breast cancer, ovarian cancer and contralateral breast cancer in BRCA1 and BRCA2 families: high cancer incidence at older age. Breast Cancer Res Treat 124:643-651 10.1007/s10549-010-0805-3.
Venkitaraman A R (2019) How do mutations affecting the breast cancer genes BRCA1 and BRCA2 cause cancer susceptibility? DNA Repair 81:102668 10.1016/j.dnarep.2019.102668.
Vidula N, Dubash T, Lawrence M S, Simoneau A, Niemierko A, Blouch E, Nagy B, Roh W, Chirn B, Reeves B A et al (2020) Identification of somatically acquired BRCA1/2 mutations by cfDNA analysis in patients with metastatic breast cancer. Clin Cancer Res 26:4852-4862 10.1158/1078-0432.CCR-20-0638.
Waks A G, Cohen O, Kochupurakkal B, Kim D, Dunn C E, Buendia Buendia J, Wander S, Helvie K, Lloyd M R, Marini L et al (2020) Reversion and non-reversion mechanisms of resistance to PARP inhibitor or platinum chemotherapy in BRCA1/2-mutant metastatic breast cancer. Ann Oncol 31:590-598 10.1016/j.annonc.2020.02.008.
Wang Q, Bergholz J S, Ding L, Lin Z, Kabraji S K, Hughes M E, He X, Xie S, Jiang T, Wang W et al (2022) STING agonism reprograms tumor-associated macrophages and overcomes resistance to PARP inhibition in BRCA1-deficient models of breast cancer. Nat Commun 13:3022 10.1038/s41467-022-30568-1.
Wang Y, Krais J J, Bernhardy A J, Nicolas E, Cai K Q, Harrell M I, Kim H H, George E, Swisher E M, Simpkins F et al (2016) RING domain-deficient BRCA1 promotes PARP inhibitor and platinum resistance. J Clin Invest 126:3145-3157 10.1172/JCI87033.
Wey J S, Fan F, Gray M J, Bauer T W, McCarty M F, Somcio R, Liu W, Evans D B, Wu Y, Hicklin D J et al (2005) Vascular endothelial growth factor receptor-1 promotes migration and invasion in pancreatic carcinoma cell lines. Cancer 104:427-438 10.1002/cncr.21145.
Wu L C, Wang Z W, Tsan J T, Spillman M A, Phung A, Xu X L, Yang M C, Hwang L Y, Bowcock A M, Baer R (1996) Identification of a RING protein that can interact in vivo with the BRCA1 gene product. Nat Genet 14:430-440 10.1038/ng1296-430.
Wu Y, Hooper A T, Zhong Z, Witte L, Bohlen P, Rafii S, Hicklin D J (2006) The vascular endothelial growth factor receptor (VEGFR-1) supports growth and survival of human breast carcinoma. Int J Cancer 119:1519-1529 10.1002/ijc.21865.
Xu X, Wagner K U, Larson D, Weaver Z, Li C, Ried T, Hennighausen L, Wynshaw-Boris A, Deng C X (1999) Conditional mutation of Brca1 in mammary epithelial cells results in blunted ductal morphogenesis and tumour formation. Nat Genet 22:37-43 10.1038/8743.
Yao J, Wu X, Zhuang G, Kasman I M, Vogt T, Phan V, Shibuya M, Ferrara N, Bais C (2011) Expression of a functional VEGFR-1 in tumor cells is a major determinant of anti-PIGF antibodies efficacy. Proc Natl Acad Sci USA 108:11590-11595 10.1073/pnas. 1109029108.
Zhang Y, Brekken R A (2022) Direct and indirect regulation of the tumor immune microenvironment by VEGF. J Leukoc Biol 111:1269-1286 10.1002/JLB.5RU0222-082R.

Many additional implementations are possible. Further implementations are within the CLAIMS.

It will be understood that implementations of treatment of PARPi resistance include but are not limited to the specific components disclosed herein, as virtually any components consistent with the intended operation of various inventions may be utilized. Accordingly, for example, it should be understood that, while the drawings and accompanying text show and describe particular invention implementations, any such implementation may comprise any shape, size, style, type, model, version, class, grade, measurement, concentration, material, weight, quantity, and/or the like consistent with the intended operation of the inventions.

In places where the description above refers to particular invention implementations, it should be readily apparent that a number of modifications may be made without departing from the spirit thereof and that these implementations may be applied to other implementations disclosed or undisclosed. The presently disclosed inventions are, therefore, to be considered in all respects as illustrative and not restrictive.

We claim:

1. A method of reversing PARP inhibitor resistance in a tumor cell, the method comprising inhibiting a signaling pathway initiated by FLT1/VEGFR1 in the tumor cell, wherein the signaling pathway initiated by FLT1/VEGFR1 is inhibited by one or more of the following:
administering to the tumor cell an AKT antagonist;
genetically inhibiting Flt1 expression in the tumor cell, such as by using a CRISPR/Cas system or RNAi system;
inhibition by a degrader system; or
by administering to the tumor cell a VEGFR antagonist, such as axitinib.

2. The method of claim 1, wherein the tumor cell is in a subject determined to be in need of treatment for PARP inhibitor resistance.

3. A method of treating cancer in a subject, the method comprising:
administering to the subject a PARP inhibitor; and
administering to the subject a therapeutic for inhibiting VEGFR activity.

4. The method of claim 3, wherein the therapeutic for inhibiting VEGFR activity is a FLT1/VEGFR1 antagonist.

5. The method of claim 4, wherein the FLT1/VEGFR1 antagonist is axitinib.

6. The method of claim 3, wherein the PARP inhibitor is talazoparib.

7. The method of claim 3, wherein the cancer is breast cancer, ovarian cancer, pancreatic cancer, or prostate cancer.

8. The method of claim 3, wherein the cancer comprises tumor cells that are deficient in a homologous recombination (HR) pathway.

9. The method of claim 3, wherein the cancer is a BRCA-mutant cancer.

10. The method of claim 3, wherein the cancer has increased expression of placental growth factor (PIGF).

11. The method of claim 3, wherein the method induces cytotoxic immune response to cancer cells in a subject, and wherein the VEGFR is VEGFR1.

12. The method of claim 3, further comprising administering immune checkpoint blockade therapy.

13. A method of detecting PARP inhibitor resistance in tumor cells comprising detecting expression or phosphorylation of a protein in a signaling pathway initiated by FLT1/VEGFR1.

14. The method of claim 13, wherein the protein in the signaling pathway initiated by FLT1/VEGFR1 is PIGF, detecting an increase in PIGF expression compared to a reference level indicates PARP inhibitor resistance in the tumor cells.

15. The method of claim 3, the method further comprising:
providing a biological sample obtained from the subject, wherein the biological sample is obtained after the subject has been administered the PARP inhibitor;
detecting expression or phosphorylation of a protein in the signaling pathway initiated by FLT1/VEGFR1, thereby detecting cancer resistant to a PARP inhibitor in the subject; and
then administering to the subject the therapeutic for inhibiting VEGFR activity upon detection of presence of PARP inhibitor resistant tumor cells in the subject.

16. The method of claim 3, the method further comprising:
first obtaining a first biological sample from the subject;
and detecting a first expression or phosphorylation level of a protein in the signaling pathway initiated by FLT1/VEGFR1;
then administering to the subject the PARP inhibitor;
obtaining a second biological sample from the subject, wherein the second biological sample is obtained after the subject has been administered the PARP inhibitor;
detecting a second expression or phosphorylation level of the protein in the signaling pathway initiated by FLT1/VEGFR1, wherein an increase in expression or phosphorylation level of the protein in the signaling pathway initiated by FLT1/VEGFR1 in the second biological sample compared to the first biological sample indicates presence of cancer cells resistant to the PARP inhibitor in the subject; and
then administering to the subject a therapeutic for inhibiting VEGFR activity upon detection of presence of cancer cells resistant to the PARP inhibitor in the subject.

17. The method of claim 16, wherein the first biological sample and the second biological sample are from a tumor.

18. The method of claim 4, the method further comprising:
first obtaining a tumor sample from the subject;
and detecting FLT1/VEGFR1 expression in the tumor sample; and
then administering the therapeutic for inhibiting VEGFR activity and the PARP inhibitor to the subject upon detection of increased expression of FLT1/VEGFR1 compared to a reference expression level.

19. The method of claim 15, further comprising administering to the subject immune checkpoint blockade therapy.

20. The method of claim 13, wherein the method screens for a therapeutic agent that reverses PARP inhibitor resistance in a tumor cell, the method further comprising:
first administering to the tumor cell a PARP inhibitor;
then detecting in the tumor cell the expression or phosphorylation of a protein in the signaling pathway initiated by FLT1/VEGFR1, wherein a first FLT1/VEGFR1 signaling pathway activity level is produced and increased expression or phosphorylation of the protein in the signaling pathway initiated by FLT1/VEGFR1 in the first FLT/VEGFR1 signaling pathway activity level compared to a reference value indicates the development of PARP inhibitor resistance in the tumor cell;
administering to the tumor cell a potential therapeutic agent; and
then detecting in the tumor cell the expression or phosphorylation of a protein in the signaling pathway initiated by FLT1/VEGFR1 after the tumor cell has been administered the potential therapeutic agent,
wherein a second FLT/VEGFR1 signaling pathway activity level is produced and reduced expression or phosphorylation of the protein in the signaling pathway initiated by FLT1/VEGFR1 in the second FLT/VEGFR1 signaling pathway activity level compared to the first FLT/VEGFR1 signaling pathway activity level indicates the potential therapeutic agent reverses PARP inhibitor resistance in the tumor cell.


SEQUENCE LISTING (6 sequences)

SEQ ID NO: 1 - DNA, 21 bp, synthetic construct
  source 1..21: mol_type = other DNA
tggctctacgaccttagactg

SEQ ID NO: 2 - DNA, 22 bp, synthetic construct
  source 1..22: mol_type = other DNA
caggtttgacttgtctgaggtt

SEQ ID NO: 3 - DNA, 21 bp, synthetic construct
  source 1..21: mol_type = other DNA
aggtcggtgtgaacggatttg

SEQ ID NO: 4 - DNA, 23 bp, synthetic construct
  source 1..23: mol_type = other DNA
tgtagaccatgtagttgaggtca

SEQ ID NO: 5 - DNA, 20 bp, synthetic construct
  source 1..20: mol_type = other DNA
cagcgcgtaaggcaagaccg

SEQ ID NO: 6 - DNA, 20 bp, synthetic construct
  source 1..20: mol_type = other DNA
caccactagcactacctccc